SEQUENCE LISTING (115 sequences)

SEQ ID NO: 1 - protein, 21 aa, Homo sapiens
  Chain A
GIVEQCCTSICSLYQLENYCN

SEQ ID NO: 2 - protein, 30 aa, Homo sapiens
  Chain B
FVNQHLCGSHLVEALYLVCGERGFFYTPKT

SEQ ID NO: 3 - protein, 21 aa, Artificial Sequence
  Chain A
GIVEQCCTSICSLEQLENYCN

SEQ ID NO: 4 - protein, 29 aa, Artificial Sequence
  Chain B
FVNQHLCGSHLVEALYLVCGERGFFYTPK

SEQ ID NO: 5 - protein, 21 aa, Artificial Sequence
  Chain A
GIVEQCCTSICSLYQLENYCN

SEQ ID NO: 6 - protein, 29 aa, Artificial Sequence
  Chain B
FVNQHLCGSHLVEALLLVCGERGFFYTPK

SEQ ID NO: 7 - protein, 21 aa, Artificial Sequence
  Chain A
GIVEQCCTSICSLYQLENYCG

SEQ ID NO: 8 - protein, 29 aa, Artificial Sequence
  Chain B
FVNQHLCGSHLVEALWLVCGERGFFYTPK

SEQ ID NO: 9 - protein, 21 aa, Artificial Sequence
  Chain A
GIVEQCCTSICSLYQLENYCN

SEQ ID NO: 10 - protein, 29 aa, Artificial Sequence
  Chain B
FVNQHLCGSHLVEALHLVCGERGFFYTPK

SEQ ID NO: 11 - protein, 21 aa, Artificial Sequence
  Chain A
GIVEQCCTSICSLYQLENYCN

SEQ ID NO: 12 - protein, 29 aa, Artificial Sequence
  Chain B
FVNQHLCGSHLVEALVLVCGERGFFYTPK

SEQ ID NO: 13 - protein, 21 aa, Artificial Sequence
  Chain A
GIVEQCCTSICSLYQLENYCN

SEQ ID NO: 14 - protein, 30 aa, Artificial Sequence
  Chain B
FVNQHLCGSHLVEALYLVCGERGFAYTPKT

SEQ ID NO: 15 - protein, 21 aa, Artificial Sequence
  Chain A
GIVEQCCTSICSLYQLENYCN

SEQ ID NO: 16 - protein, 29 aa, Artificial Sequence
  Chain B
FVNQHLCGSHLVEALYLVCGERGFAYTPK

SEQ ID NO: 17 - protein, 21 aa, Artificial Sequence
  Chain A
GIVEQCCTSICSLYQLENYCN

SEQ ID NO: 18 - protein, 29 aa, Artificial Sequence
  Chain B
FVNQHLCGSHLVEALYLVCGERGFEYTPK

SEQ ID NO: 19 - protein, 21 aa, Artificial Sequence
  Chain A
GIVEQCCTSICSLYQLENYCN

SEQ ID NO: 20 - protein, 29 aa, Artificial Sequence
  Chain B
FVNQHLCGSHLVEALYLVCGERGFHYTPK

SEQ ID NO: 21 - protein, 21 aa, Artificial Sequence
  Chain A
GIVEQCCTSICSLYQLENYCN

SEQ ID NO: 22 - protein, 29 aa, Artificial Sequence
  Chain B
FVNQHLCGSHLVEALYLVCGERGFLYTPK

SEQ ID NO: 23 - protein, 21 aa, Artificial Sequence
  Chain A
GIVEQCCTSICSLYQLENYCN

SEQ ID NO: 24 - protein, 29 aa, Artificial Sequence
  Chain B
FVNQHLCGSHLVEALYLVCGERGFVYTPK

SEQ ID NO: 25 - protein, 21 aa, Artificial Sequence
  Chain A
GIVEQCCTSICSLYQLENYCN

SEQ ID NO: 26 - protein, 29 aa, Artificial Sequence
  Chain B
FVNQHLCGSHLVEALHLVCGERGFHYTPK

SEQ ID NO: 27 - protein, 21 aa, Artificial Sequence
  Chain A
GIVEQCCTSICSLYQLENYCG

SEQ ID NO: 28 - protein, 29 aa, Artificial Sequence
  Chain B
FVNQHLCGSHLVEALWLVCGERGFHYTPK

SEQ ID NO: 29 - protein, 21 aa, Artificial Sequence
  Chain A
GIVEQCCTSICSLYQLENYCG

SEQ ID NO: 30 - protein, 29 aa, Artificial Sequence
  Chain B
FVNQHLCGSHLVEALWLVCGERGFWYTPK

SEQ ID NO: 31 - protein, 21 aa, Artificial Sequence
  Chain A
GIVEQCCTSICSLEQLENYCN

SEQ ID NO: 32 - protein, 29 aa, Artificial Sequence
  Chain B
FVNQHLCGSHLVEALHLVCGERGFFYTPK

SEQ ID NO: 33 - protein, 21 aa, Artificial Sequence
  Chain A
GIVEQCCTSICSLEQLENYCG

SEQ ID NO: 34 - protein, 29 aa, Artificial Sequence
  Chain B
FVNQHLCGSHLVEALWLVCGERGFFYTPK

SEQ ID NO: 35 - protein, 21 aa, Artificial Sequence
  Chain A
GIVEQCCTSICSLEQLENYCN

SEQ ID NO: 36 - protein, 29 aa, Artificial Sequence
  Chain B
FVNQHLCGSHLVEALILVCGERGFFYTPK

SEQ ID NO: 37 - protein, 21 aa, Artificial Sequence
  Chain A
GIVEQCCTSICSLEQLENYCN

SEQ ID NO: 38 - protein, 29 aa, Artificial Sequence
  Chain B
FVNQHLCGSHLVEALVLVCGERGFFYTPK

SEQ ID NO: 39 - protein, 21 aa, Artificial Sequence
  Chain A
GIVEQCCTSICSLEQLENYCN

SEQ ID NO: 40 - protein, 29 aa, Artificial Sequence
  Chain B
FVEQHLCGSHLVEALVLVCGERGFFYTPK

SEQ ID NO: 41 - protein, 21 aa, Artificial Sequence
  Chain A
GIVEQCCTSICSLEQLENYCN

SEQ ID NO: 42 - protein, 29 aa, Artificial Sequence
  Chain B
FVNQHLCGSHLVEALYLVCGERGFHYTPK

SEQ ID NO: 43 - protein, 21 aa, Artificial Sequence
  Chain A
GIVEQCCTSICSLEQLENYCN

SEQ ID NO: 44 - protein, 29 aa, Artificial Sequence
  Chain B
FVNQHLCGSHLVEALYLVCGERGFIYTPK

SEQ ID NO: 45 - protein, 21 aa, Artificial Sequence
  Chain A
GIVEQCCTSICSLEQLENYCG

SEQ ID NO: 46 - protein, 29 aa, Artificial Sequence
  Chain B
FVNQHLCGSHLVEALYLVCGERGFWYTPK

SEQ ID NO: 47 - protein, 21 aa, Artificial Sequence
  Chain A
GIVEQCCTSICSLEQLENYCN

SEQ ID NO: 48 - protein, 29 aa, Artificial Sequence
  Chain B
FVNQHLCGSHLVEALYLVCGERGFVYTPK

SEQ ID NO: 49 - protein, 21 aa, Artificial Sequence
  Chain A
GIVEQCCTSICSLEQLENYCG

SEQ ID NO: 50 - protein, 29 aa, Artificial Sequence
  Chain B
FVEQHLCGSHLVEALYLVCGERGFVYTPK

SEQ ID NO: 51 - protein, 21 aa, Artificial Sequence
  Chain A
GIVEQCCTSICSLEQLENYCN

SEQ ID NO: 52 - protein, 29 aa, Artificial Sequence
  Chain B
FVNQHLCGSHLVEALELVCGERGFHYTPK

SEQ ID NO: 53 - protein, 21 aa, Artificial Sequence
  Chain A
GIVEQCCTSICSLEQLENYCN

SEQ ID NO: 54 - protein, 29 aa, Artificial Sequence
  Chain B
FVNQHLCGSHLVEALHLVCGERGFAYTPK

SEQ ID NO: 55 - protein, 21 aa, Artificial Sequence
  Chain A
GIVEQCCTSICSLEQLENYCN

SEQ ID NO: 56 - protein, 29 aa, Artificial Sequence
  Chain B
FVNQHLCGSHLVEALHLVCGERGFHYTPK

SEQ ID NO: 57 - protein, 21 aa, Artificial Sequence
  Chain A
GIVEQCCTSICSLEQLENYCN

SEQ ID NO: 58 - protein, 29 aa, Artificial Sequence
  Chain B
FVNQHLCGSHLVEALILVCGERGFIYTPK

SEQ ID NO: 59 - protein, 21 aa, Artificial Sequence
  Chain A
GIVEQCCTSICSLEQLENYCN

SEQ ID NO: 60 - protein, 29 aa, Artificial Sequence
  Chain B
FVEQHLCGSHLVEALILVCGERGFIYTPK

SEQ ID NO: 61 - protein, 21 aa, Artificial Sequence
  Chain A
GIVEQCCTSICSLEQLENYCG

SEQ ID NO: 62 - protein, 29 aa, Artificial Sequence
  Chain B
FVEQHLCGSHLVEALILVCGERGFWYTPK

SEQ ID NO: 63 - protein, 21 aa, Artificial Sequence
  Chain A
GIVEQCCTSICSLEQLENYCN

SEQ ID NO: 64 - protein, 29 aa, Artificial Sequence
  Chain B
FVNQHLCGSHLVEALILVCGERGFVYTPK

SEQ ID NO: 65 - protein, 21 aa, Artificial Sequence
  Chain A
GIVEQCCTSICSLEQLENYCG

SEQ ID NO: 66 - protein, 29 aa, Artificial Sequence
  Chain B
FVEQHLCGSHLVEALILVCGERGFVYTPK

SEQ ID NO: 67 - protein, 21 aa, Artificial Sequence
  Chain A
GIVEQCCTSICSLEQLENYCN

SEQ ID NO: 68 - protein, 29 aa, Artificial Sequence
  Chain B
FVNQHLCGSHLVEALLLVCGERGFAYTPK

SEQ ID NO: 69 - protein, 21 aa, Artificial Sequence
  Chain A
GIVEQCCTSICSLEQLENYCN

SEQ ID NO: 70 - protein, 29 aa, Artificial Sequence
  Chain B
FVNQHLCGSHLVEALVLVCGERGFIYTPK

SEQ ID NO: 71 - protein, 21 aa, Artificial Sequence
  Chain A
GIVEQCCTSICSLEQLENYCG

SEQ ID NO: 72 - protein, 29 aa, Artificial Sequence
  Chain B
FVNQHLCGSHLVEALVLVCGERGFWYTPK

SEQ ID NO: 73 - protein, 21 aa, Artificial Sequence
  Chain A
GIVEQCCTSICSLEQLENYCG

SEQ ID NO: 74 - protein, 29 aa, Artificial Sequence
  Chain B
FVEQHLCGSHLVEALVLVCGERGFWYTPK

SEQ ID NO: 75 - protein, 21 aa, Artificial Sequence
  Chain A
GIVEQCCTSICSLEQLENYCN

SEQ ID NO: 76 - protein, 29 aa, Artificial Sequence
  Chain B
FVNQHLCGSHLVEALVLVCGERGFVYTPK

SEQ ID NO: 77 - protein, 21 aa, Artificial Sequence
  Chain A
GIVEQCCTSICSLEQLENYCN

SEQ ID NO: 78 - protein, 29 aa, Artificial Sequence
  Chain B
FVEQHLCGSHLVEALVLVCGERGFVYTPK

SEQ ID NO: 79 - protein, 21 aa, Artificial Sequence
  Chain A
GIVEQCCTSICSLEQLENYCG

SEQ ID NO: 80 - protein, 29 aa, Artificial Sequence
  Chain B
FVEQHLCGSHLVEALVLVCGERGFVYTPK

SEQ ID NO: 81 - protein, 21 aa, Bos
  Chain A
GIVEQCCASVCSLYQLENYCN

SEQ ID NO: 82 - protein, 30 aa, Bos
  Chain B
FVNQHLCGSHLVEALYLVCGERGFFYTPKA

SEQ ID NO: 83 - protein, 21 aa, Sus
  Chain A
GIVEQCCTSICSLYQLENYCN

SEQ ID NO: 84 - protein, 30 aa, Sus; identical to SEQ ID NO: 82
  Chain B

SEQ ID NO: 85 - protein, 29 aa, Artificial Sequence
  Chain B
FVNQHLCGSHLVEALYLVCGERGFLYTPK

SEQ ID NO: 86 - protein, 29 aa, Artificial Sequence
  Chain B
FVNQHLCGSHLVEALYLVCGERGFVYTPK

SEQ ID NO: 87 - protein, 29 aa, Artificial Sequence
  Chain B
FVNQHLCGSHLVEALYLVCGERGFIYTPK

SEQ ID NO: 88 - protein, 29 aa, Artificial Sequence
  Chain B
FVNQHLCGSHLVEALYLVCGERGFVYTPK

SEQ ID NO: 89 - protein, 29 aa, Artificial Sequence
  Chain B
FVEQHLCGSHLVEALYLVCGERGFVYTPK

SEQ ID NO: 90 - protein, 29 aa, Artificial Sequence
  Chain B
FVNQHLCGSHLVEALILVCGERGFIYTPK

SEQ ID NO: 91 - protein, 29 aa, Artificial Sequence
  Chain B
FVEQHLCGSHLVEALILVCGERGFIYTPK

SEQ ID NO: 92 - protein, 29 aa, Artificial Sequence
  Chain B
FVNQHLCGSHLVEALILVCGERGFVYTPK

SEQ ID NO: 93 - protein, 29 aa, Artificial Sequence
  Chain B
FVEQHLCGSHLVEALILVCGERGFVYTPK

SEQ ID NO: 94 - protein, 29 aa, Artificial Sequence
  Chain B
FVNQHLCGSHLVEALVLVCGERGFIYTPK

SEQ ID NO: 95 - protein, 29 aa, Artificial Sequence
  Chain B
FVNQHLCGSHLVEALVLVCGERGFVYTPK

SEQ ID NO: 96 - protein, 29 aa, Artificial Sequence
  Chain B
FVEQHLCGSHLVEALVLVCGERGFVYTPK

SEQ ID NO: 97 - protein, 29 aa, Artificial Sequence
  Chain B
FVEQHLCGSHLVEALVLVCGERGFVYTPK

SEQ ID NO: 98 - protein, 44 aa, Artificial Sequence
  Lixisenatide
HGEGTFTSDLSKQMEEEAVRLFIEWLKNGGPSSGAPPSKKKKKK

SEQ ID NO: 99 - protein, 39 aa, Artificial Sequence
  Exenatide
HGEGTFTSDLSKQMEEEAVRLFIEWLKNGGPSSGAPPPS

SEQ ID NO: 100 - protein, 31 aa, Artificial Sequence
  Semaglutide
  MOD_RES 2: Aib
HAEGTFTSDVSSYLEGQAAKEFIAWLVRGRG

SEQ ID NO: 101 - protein, 31 aa, Artificial Sequence
  Dulaglutide
HAEGTFTSDVSSYLEGQAAKEFIAWLVKGRG

SEQ ID NO: 102 - protein, 21 aa, Homo sapiens
  human insulin , A chain
GIVEQCCTSICSLYQLENYCN

SEQ ID NO: 103 - protein, 30 aa, Homo sapiens; identical to SEQ ID NO: 2
  human insulin , B chain

SEQ ID NO: 104 - protein, 21 aa, Artificial Sequence
  insulin analog 1 , A chain
GIVEQCCTSICSLEQLENYCN

SEQ ID NO: 105 - protein, 29 aa, Artificial Sequence
  insulin analog 1 , B chain
FVNQHLCGSHLVEALHLVCGERGFHYTPK

SEQ ID NO: 106 - protein, 9 aa, Artificial Sequence
  MOD_RES 1: Xaa is any naturally occurring amino acid
  Linker peptide
  MOD_RES 2..8: Xaa is any naturally occurring amino acid or is absent
XXXXXXXXR

SEQ ID NO: 107 - protein, 25 aa, Artificial Sequence
  N-terminally extended A-chain
TEGRGIVEQCCTSICSLEQLENYCN

SEQ ID NO: 108 - protein, 54 aa, Artificial Sequence
  Proinsulin variant
FVNQHLCGSHLVEALYLVCGERGFVYTPKTEGRGIVEQCCTSICSLEQLENYCN

SEQ ID NO: 109 - protein, 21 aa, Artificial Sequence
  A-chain
  MOD_RES 14: Xaa is glutamic acid (Glu), aspartic acid (Asp) or histidine (His)
GIVEQCCTSICSLXQLENYCN

SEQ ID NO: 110 - protein, 29 aa, Artificial Sequence
  B-chain
  MOD_RES 16: Xaa is tyrosine (Tyr), valine (Val), isoleucine (Ile), leucine (Leu), alanine (Ala) or histidine (His)
  MOD_RES 25: Xaa is phenylalanine (Phe), valine (Val), isoleucine (Ile), leucine (Leu), alanine (Ala) or histidine (His)
FVNQHLCGSHLVEALXLVCGERGFXYTPK

SEQ ID NO: 111 - protein, 59 aa, Artificial Sequence
  Proinsulin
  MOD_RES 16: Xaa is is tyrosine (Tyr), valine (Val), isoleucine (Ile), leucine (Leu), alanine (Ala) or histidine (His)
  MOD_RES 25: Xaa is phenylalanine (Phe), valine (Val), isoleucine (Ile), leucine (Leu), alanine (Ala) or histidine (His)
  MOD_RES 30: Xaa is any naturally occuring amino acid
  MOD_RES 31..37: Xaa is any naturally occurring amino acid or is absent
  MOD_RES 52: Xaa is glutamic acid (Glu), aspartic acid (Asp) or histidine (His)
FVNQHLCGSHLVEALXLVCGERGFXYTPKXXXXXXXXRGIVEQCCTSICSLXQLENYCN

SEQ ID NO: 112 - protein, 4 aa, Artificial Sequence
  Linker peptide
TEGR

SEQ ID NO: 113 - protein, 30 aa, Artificial Sequence
  MOD_RES 1: Xaa is any naturally occurring amino acid
  N-terminally extended A-chain
  MOD_RES 2..8: Xaa is any naturally occurring amino acid or is absent
  MOD_RES 23: Xaa is glutamic acid (Glu), aspartic acid (Asp) or histidine (His)
XXXXXXXXRGIVEQCCTSICSLXQLENYCN

SEQ ID NO: 114 - protein, 4 aa, Artificial Sequence
  MOD_RES 1: Xaa is any naturally occurring amino acid
  Linker peptide
  MOD_RES 2..3: Xaa is any naturally occurring amino acid or is absent
XXXR

SEQ ID NO: 115 - protein, 4 aa, Artificial Sequence
  Linker peptide
  MOD_RES 2..3: Xaa is any naturally occurring amino acid or is absent
TXXR

(19) United States
(12) Patent Application Publication
(10) Pub. No.: US 2023/0346949 A1
(43) Pub. Date: Nov. 2, 2023

(54) A METHOD OF FORMING A CONJUGATE OF A SULFONAMIDE AND A POLYPEPTIDE

(71) Applicant: SANOFI, Paris (FR)

(72) Inventors: Thomas BOEHME, Frankfurt am Main (DE); Marisa RAEDISCH, Frankfurt am Main (DE); Manfred GERKEN, Frankfurt am Main (DE); Oliver STEYER, Frankfurt am Main (DE); Oliver BOSCHEINEN, Frankfurt am Main (DE); Daniel SCHEPS, Frankfurt am Main (DE); Hermut WEHLAN, Frankfurt am Main (DE)

(21) Appl. No.: 17/778,935

(22) PCT Filed: Dec. 10, 2020
(86) PCT No.: PCT/EP2020/085415

§ 371 Date: May 23, 2022

(30) Foreign Application Priority Data
Dec. 10, 2019 (EP) 19306610.7

(51) Int. Cl. A61K 47/54; A61K 47/65; C07K 14/62

(52) U.S. Cl. CPC A61K 47/545; A61K 47/65; C07K 14/62

(57) ABSTRACT

A method of forming a conjugate of a sulfonamide and a polypeptide, the method comprising: a) Providing an activated sulfonamide, wherein the activated sulfonamide corresponds to Formula (I); b) Providing an aqueous solution of a polypeptide having a free amino group, wherein the aqueous solution optionally comprises an alcohol; c) Contacting the aqueous solution of b) with the activated sulfonamide of a); and d) Reacting the activated sulfonamide with the polypeptide having a free amino group, obtaining a solution comprising the conjugate of a sulfonamide and a polypeptide, wherein the sulfonamide is covalently bonded to the polypeptide. Also associated conjugates, processes, procedures, proinsulins and the like.

### BACKGROUND

Polypeptides are used in various application fields. Since polypeptides have several functional groups, for example, an NH₂ group at the N terminus, a COOH group at the C terminus and normally one or more further functional groups directly bound to the polypeptide chain or to side chains, coupling with other compounds is complicated. Normally, byproducts are formed which reduce the yield of the desired product and which are hard to separate from the desired product.

Human insulin is a polypeptide of 51 amino acid residues, which are divided into 2 amino acid chains: the A chain having 21 amino acid residues and the B chain having 30 amino acid residues. The chains are connected to one another by means of 2 disulfide bridges. A third disulfide bridge exists between the cysteines at position 6 and 11 of the A chain. Some products in current use for the treatment of diabetes mellitus are insulin analogs, i.e. insulin variants whose sequence differs from that of human insulin by one or more amino acid substitutions in the A chain and/or in the B chain.

Like many other peptide hormones, human insulin has a short half-life in vivo. Thus, it is administered frequently which is associated with discomfort for the patient. Therefore, insulin analogs are desired which have an increased half-life in vivo and, thus, a prolonged duration of action.

WO 2008/034881A1 (Novo Nordisk, Nielsen) discloses protease stabilized insulin analogs.

In another approach, a long chain fatty acid group is conjugated to the epsilon amino group of LysB29 of insulin. The presence of this group allows the attachment of the insulin to serum albumin by noncovalent, reversible binding. As a consequence, this insulin analog has a significantly prolonged time-action profile relative to human insulin (see e.g. Mayer et al., Inc. Biopolymers (Pept Sci) 88: 687-713, 2007; or WO 2009/115469 A1 (Novo Nordisk, Madsen)). Another conjugate comprising an insulin analog and a covalently attached functional group which allows the attachment of the insulin to serum albumin by noncovalent, reversible binding is disclosed in WO 2017/032798A1 (Novo Nordisk, Madsen): here, an acylated analogue of human insulin is described, which insulin analog is derivatized by acylation of the epsilon amino group of the lysine residue at the A22 position with a group [Acyl]-[Linker]- wherein the Linker group is an amino acid chain composed of from 1 to 10 amino acid residues selected from gGlu (gamma glutamic acid residue) and/or OEG (residue of 8-amino-3,6-dioxaoctanoic acid).

One obstacle in synthesis of such conjugates, i.e. wherein an insulin analog is coupled with another molecule, which in turn allows the attachment of the insulin (analog) to serum albumin by noncovalent, reversible binding, is that the coupling step often results in poor yields and that a lot of work-up and purification steps are required which lower the yield further.

### SUMMARY

Provided herein is a method of forming a conjugate of a sulfonamide and a polypeptide, which enables higher overall yields in conjugate synthesis, i.e. yields of more than 20%, or more than 30%, or more than 40%, or more than 50%, based on the polypeptide used. The method is described herein below in section A.

Further provided is a process for generating a conjugate of an albumin binder and an insulin polypeptide comprising: a) providing a proinsulin comprising from N- to C-terminus an insulin B-chain, a linker peptide and an insulin A-chain, b) cleaving the proinsulin provided in step a) with a first protease between the last amino acid of the insulin B-chain and the first amino acid of the linker peptide, thereby generating an insulin precursor, said insulin precursor comprising the insulin B-chain and an N-terminally extended A-chain comprising the linker peptide and the A-chain, c) contacting said insulin precursor with an albumin binder, thereby generating a conjugate of an albumin binder and the insulin precursor, d) cleaving the N-terminally extended A-chain of said insulin precursor comprised by the conjugate with a second protease between the last amino acid of the linker peptide and the first amino acid of the A-chain, thereby generating a conjugate of an albumin binder and a mature insulin. The process is described herein below in section B.

### DETAILED DESCRIPTION

Section A: Method of Forming a Conjugate of a Sulfonamide and a Polypeptide

In order to increase the yield in conjugate synthesis, the number of steps required and the number as well as the ratio of byproducts plays a major role. Accordingly, there is a need for synthetic routes, which have a reduced number of steps and which at least have a more favorable ratio of desired product to undesired byproducts.

Surprisingly, it was found that using a specifically activated sulfonamide for the coupling reaction with the polypeptide helps to improve the ratio of desired product to undesired byproducts. Further, the use of a polypeptide precursor in combination with the use of specific enzymes enables a so called “one pot” reaction, i.e. coupling of the activated sulfonamide and cleavage of the polypeptide precursor to reach the final polypeptide can be done in one reaction vessel without the need for separation or intermediate purification steps. The use of additional protection groups for the sulfonamide can be avoided, this also reducing the need for intermediate separation or purification steps. The desired product, that is the conjugate of the sulfonamide and a polypeptide, can be obtained in yields of 50% or more.

Accordingly, provided herein is in a first aspect a method of forming a conjugate of a sulfonamide and a polypeptide, the method comprising:
a) Providing an activated sulfonamide, wherein the activated sulfonamide corresponds to Formula (I):

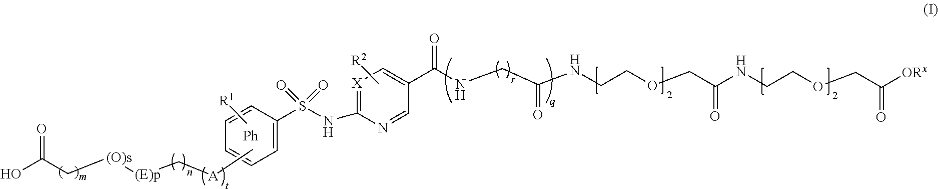

wherein
A is selected from the group consisting of oxygen atom, —CH₂CH₂— group, —OCH₂— group and —CH₂O— group;
E represents a —C₆H₃R— group with R being a hydrogen atom or a halogen atom, wherein the halogen atom is selected from the group consisting of fluorine, chlorine, bromine and iodine atom;
X represents a nitrogen atom or a —CH— group;
m is an integer in the range from 5 to 17;
n is zero or an integer in the range from 1 to 3;
p is zero or 1;
q is zero or 1;
r is an integer in the range from 1 to 6;
s is zero or 1;
t is zero or 1;
R¹ represents at least one residue selected from the group of hydrogen atom, halogen atom, C1 to C3 alkyl group and halogenated C1 to C3 alkyl group;
R² represents at least one residue selected from the group of hydrogen atom, halogen atom, C1 to C3 alkyl group and halogenated C1 to C3 alkyl group;
R^{x} represents an activation group;
wherein the combination of s being 1, p being zero, n being zero, A being an oxygen atom and t being 1 is excluded for Formula (I);
b) Providing an aqueous solution of a polypeptide, wherein the aqueous solution optionally comprises an alcohol;
c) Contacting the aqueous solution of b) with the activated sulfonamide of a); and
d) Reacting the activated sulfonamide with the polypeptide, obtaining a solution comprising the conjugate of a sulfonamide and the active pharmaceutical ingredient or the diagnostic compound, wherein the sulfonamide is covalently bonded to the polypeptide.

In at least one embodiment, the activated sulfonamide of Formula (I) is covalently bound to the polypeptide in that the terminal carboxy group carrying the R^{x} group in the non-coupled state of the activated sulfonamide of Formula (I) is covalently bond to a suitable functional group of the polypeptide, for example to an amino group or a hydroxyl group of the polypeptide.

A “polypeptide” is a peptide which comprises at least 2 amino acid residues. In some embodiments, the peptide comprises at least 10 amino acid residues, or at least 20 amino acid residues. In some embodiments, the peptide comprises not more than 1000 amino acid residues, such as not more than 500 amino acid residues, for example not more than 100 amino acid residues.

As used herein, the term “polypeptide” includes any diagnostic chemical or biological polypeptide, pharmaceutically active chemical or biological polypeptide and any pharmaceutically acceptable salt of a diagnostic or pharmaceutically active polypeptide and any mixture thereof, that provides some diagnostic effect or some pharmacologic effect and is used for diagnosing, treating or preventing a condition.

The “polypeptide” is a mature polypeptide or a precursor thereof.

In at least one embodiment, the polypeptide is selected from the group consisting of antidiabetic polypeptide, antiobesity polypeptide, appetite regulating polypeptide, antihypertensive polypeptide, polypeptide for the treatment and/or prevention of complications resulting from or associated with diabetes, polypeptides for the treatment and/or prevention of complications and disorders resulting from or associated with obesity, and a precursor of any one of those polypeptides.

In at least one embodiment of the method, the polypeptide is an antidiabetic polypeptide or a precursor thereof. In some embodiments, the polypeptide is GLP-1, GLP-1 analog, GLP-1 receptor agonist; dual GLP-1 receptor/glucagon receptor agonist; human FGF21, FGF21 analog, FGF21 derivative; insulin (for example human insulin), insulin analog, insulin derivative, or a precursor of any one of those polypeptides.

According to at least one embodiment of the method, the polypeptide is selected from the group consisting of insulin, insulin analog, GLP-1, and GLP-1 analog (for example GLP(-1) agonist) and a precursor of any one of those polypeptides.

As used herein, the terms “GLP-1 analog” refer to a polypeptide which has a molecular structure which formally can be derived from the structure of a naturally occurring glucagon-like-peptide-1 (GLP-1), for example that of human GLP-1, by deleting and/or exchanging at least one amino acid residue occurring in the naturally occurring GLP-1 and/or adding at least one amino acid residue. The added and/or exchanged amino acid residue can either be codable amino acid residues or other naturally occurring residues or purely synthetic amino acid residues.

As used herein, the term “GLP(-1) receptor agonist” refers to analogs of GLP(-1), which activate the glucagon-like-peptide-1-rezeptor (GLP-1-rezeptor). Examples of GLP(-1) agonists include, but are not limited to, the following: lixisenatide, exenatide/exendin-4, semaglutide, taspoglutide, albiglutide, dulaglutide.

Lixisenatide has the following amino acid sequence (SEQ ID NO: 98):
His-Gly-Glu-Gly-
Thr-Phe-Thr-Ser-Asp-Leu-Ser-Lys-Gln-Met-Glu-Glu-Glu-Ala-Val-Arg-Leu-Phe-Ile-
Glu-Trp-Leu-Lys-Asn-Gly-Gly-Pro-Ser-Ser-Gly-Ala-Pro-Pro-Ser-Lys-Lys-Lys-Lys-
Lys-Lys-NH₂
Exenatide has the following amino acid sequence (SEQ ID NO: 99):
His-Gly-Glu-Gly-Thr-Phe-Thr-Ser-Asp-Leu-Ser-Lys-Gln-Met-Glu-Glu-Glu-Ala-Val-Arg-Leu-
Phe-Ile-Glu-Trp-Leu-Lys-Asn-Gly-Gly-Pro-Ser-Ser-Gly-Ala-Pro-Pro-Pro-Ser-NH₂
Semaglutide-albumin binder coupled to Lys(20) has the following amino acid sequence
(SEQ ID NO: 100):
His-Aib-Glu-Gly-Thr-Phe-Thr-Ser-Asp-Val-Ser-Ser-Tyr-Leu-Glu-Gly-Gln-Ala-Ala-Lys(AEEAc-
AEEAc-γ-Glu-17-carboxyheptadecanoyl)-Glu-Phe-Ile-Ala-Trp-Leu-Val-Arg-Gly-Arg-Gly
Dulaglutide(GLP1(7-37) coupled via peptidic linker to an fc-fragment) has the following
amino acid sequence (SEQ ID NO: 101):
His-Ala-Glu-Gly-Thr-Phe-Thr-Ser-Asp-Val-Ser-Ser-Tyr-Leu-Glu-Gly-Gln-Ala-Ala-Lys-Glu-
Phe-Ile-Ala-Trp-Leu-Val-Lys-Gly-Arg-Gly

As used herein, the term “FGF-21” means “fibroblast growth factor 21”. FGF-21 compounds may be human FGF-21, an analog of FGF-21 (referred to “FGF-21 analog”) or a derivative of FGF-21 (referred to “FGF-21 derivative”).

According to at least one embodiment of the method, the polypeptide is insulin, an insulin analog, or a precursor of insulin or of insulin analog. The expression “insulin analog” as used herein refers to a peptide which has a molecular structure which formally can be derived from the structure of a naturally occurring insulin (herein also referred to as “parent insulin”, e.g. human insulin) by deleting and/or substituting at least one amino acid residue occurring in the naturally occurring insulin and/or adding at least one amino acid residue. The added and/or exchanged amino acid residue can either be codable amino acid residues or other naturally occurring residues or purely synthetic amino acid residues. The analog as referred to herein is capable of lowering blood glucose levels in vivo, such as in a human subject.

In at least one embodiment, “insulin analog” refers to an analog of human insulin (human insulin analog), whose sequence differs from that of human insulin by one or more amino acid substitutions in the A chain and/or in the B chain.

In some embodiments, the insulin, insulin analog, or the precursor of insulin or of insulin analog has an epsilon amino group of a lysine present in the insulin or insulin analog or precursor of insulin or of insulin analog or is the N-terminal amino group of the B chain of the insulin, the insulin analog, the precursor of insulin or the precursor of insulin analog. For example, the insulin or insulin analog or precursor thereof has one lysine in the A chain and/or B chain. In some embodiments, the insulin, insulin analog, precursor of insulin or precursor of insulin analog has one lysine in the A and in the B chain. In some embodiments, the activated sulfonamide of Formula (I) is covalently bond to lysine, for example to the epsilon amino group of the lysine of the polypeptide in that the terminal carboxy group carrying the R^{x} group in the pre-coupled state of the activated sulfonamide of Formula (I) forms an amide bond with the amino group.

In some embodiments, the insulin analog provided herein comprises two peptide chains, an A-chain and a B-chain. Typically, the two chains are connected by disulfide bridges between cysteine residues. For example, in some embodiments, insulin analogs provided herein comprise three disulfide bridges: one disulfide bridge between the cysteines at position A6 and A11, one disulfide bridge between the cysteine at position A7 of the A-chain and the cysteine at position B7 of the B-chain, and one between the cysteine at position A20 of the A-chain and the cysteine at position B19 of the B-chain. Accordingly, insulin analogs provided herein may comprise cysteine residues at positions A6, A7, A11, A20, B7 and B19.

Mutations of insulin, i.e. mutations of a parent insulin, are indicated herein by referring to the chain, i.e. either the A-chain or the B-chain of the analog, the position of the mutated amino acid residue in the A- or B-chain (such as A14, B16 and B25), and the three letter code for the amino acid substituting the native amino acid in the parent insulin. The term “desB30” refers to an analog lacking the B30 amino acid from the parent insulin (i.e. the amino acid at position B30 is absent). For example, Glu(A14)Ile(B16)desB30 human insulin, is an analog of human insulin in which the amino acid residue at position 14 of the A-chain (A14) of human insulin is substituted with glutamic acid, the amino acid residue at position 16 of the B-chain (B16) is substituted with isoleucine, and the amino acid at position 30 of the B chain is deleted (i.e. is absent).

Insulin analogs that can be used in the method described herein comprise at least one mutation (substitution, deletion, or addition of an amino acid) relative to parent insulin. The term “at least one”, as used herein means one, or more than one, such as “at least two”, “at least three”, “at least four”, “at least five”, etc. In some embodiments, the insulin analogs provided herein comprise at least one mutation in the B-chain and at least one mutation in the A-chain. In a further embodiment, the insulin analogs provided herein comprise at least two mutations in the B-chain and at least one mutation in the A-chain.

In some embodiments, the insulin analog comprises an A chain and a B chain, wherein the A chain comprises at least one mutation relative to the A chain of the parent insulin (such as human insulin) and/or wherein the B chain comprises at least one mutation relative to the parent insulin (such as human insulin). For example, the at least one mutation relative to the A chain of human insulin is a substitution at position A14, such as a substitution with an amino acid selected from the group consisting of glutamic acid (Glu), aspartic acid (Asp) and histidine (His), and/or a substitution at position A21, such as a substitution with glycine (Gly). For example, the mutation relative to the B chain of human insulin may be a substitution at position B16, such as a substitution with an amino acid selected from the group consisting of valine (Val), isoleucine (Ile), leucine (Leu), alanine (Ala) or histidine (His), a substitution at position B25, such as a substitution with valine (Val), isoleucine (Ile), leucine (Leu), alanine (Ala) or histidine (His), and/or a deletion at position B30.

In some embodiments, the insulin analog comprises a deletion at position B30. In some embodiments, the insulin analog may comprise a substitution at position B16, a deletion at position B30 and a substitution at position A14. In some embodiments, the insulin analog may comprise a substitution at position B25, a deletion at position B30 and a substitution at position A14. In some embodiments, the insulin analog may comprise a substitution at position B16, a substitution at position B25, a deletion at position B30 and a substitution at position A14.

The insulin analogs provided herein may comprise mutations in addition to the mutations above. In some embodiments, the number of mutations does not exceed a certain number. In some embodiments, the insulin analogs comprise less than twelve mutations (i.e. deletions, substitution, additions) relative to the parent insulin. In another embodiment, the analog comprises less than ten mutations relative to the parent insulin. In another embodiment, the analog comprises less than eight mutations relative to the parent insulin. In another embodiment, the analog comprises less than seven mutations relative to the parent insulin. In another embodiment, the analog comprises less than six mutations relative to the parent insulin. In another embodiment, the analog comprises less than five mutations relative to the parent insulin. In another embodiment, the analog comprises less than four mutations relative to the parent insulin. In another embodiment, the analog comprises less than three mutations relative to the parent insulin.

The expression “parent insulin” as used herein refers to naturally occurring insulin, i.e. to an unmutated, wild-type insulin. In some embodiments, the parent insulin is animal insulin, such as mammalian insulin. For example, the parent insulin may be human insulin, porcine insulin, or bovine insulin.

In some embodiments, the parent insulin is human insulin. The sequence of human insulin is well known in the art and shown in Table 1 in the Example section. Human insulin comprises an A chain having an amino acid sequence as shown in SEQ ID NO: 1 (GIVEQCCTSICSLYQLENYCN) and a B chain having an amino acid sequence as shown in SEQ ID NO: 2 (FVNQHLCGSHLVEALYLVCGERGFFYTPKT).

In another embodiment, the parent insulin is bovine insulin. The sequence of bovine insulin is well known in the art. Bovine insulin comprises an A chain having an amino acid sequence as shown in SEQ ID NO: 81 (GIVEQCCASVCSLYQLENYCN) and a B chain having an amino acid sequence as shown in SEQ ID NO: 82 (FVNQHLCGSHLVEALYLVC-GERGFFYTPKA).

In another embodiment, the parent insulin is porcine insulin. The sequence of porcine insulin is well known in the art. Porcine insulin comprises an A chain having an amino acid sequence as shown in SEQ ID NO: 83 (GIVEQCCTSICSLYQLENYCN) and a B chain having an amino acid sequence as shown in SEQ ID NO: 84 (FVNQHLCGSHLVEALYLVC GERGFFYTPKA).

Human, bovine, and porcine insulin comprises three disulfide bridges: one disulfide bridge between the cysteines at position A6 and A11, one disulfide bridge between the cysteine at position A7 of the A-chain and the cysteine at position B7 of the B-chain, and one between the cysteine at position A20 of the A-chain and the cysteine at position B19 of the B-chain.

The term “mature insulin” as referred to herein shall include parent insulin, such as human insulin, and insulin analogs. In some embodiments, the mature insulin is an insulin analog, such as an insulin analog listed in Table 1 of the Examples section. For example, the insulin analog may be insulin analog 24 of Table.

The insulin analogs provided herein typically have an insulin receptor binding affinity which is reduced as compared to the insulin receptor binding affinity of the corresponding parent insulin, e.g. of human insulin.

The insulin receptor can be any mammalian insulin receptor, such as a bovine, porcine or human insulin receptor. In some embodiments, the insulin receptor is a human insulin receptor, e.g. human insulin receptor isoform A or human insulin receptor isoform B (which was used in the Examples section).

Advantageously, the human insulin analogs provided herein have a significantly reduced binding affinity to the human insulin receptor as compared to the binding affinity of human insulin to the human insulin receptor (see Examples). Thus, the insulin analogs have a very low clearance rate, i.e. a very low insulin-receptor-mediated clearance rate.

In some embodiments, the insulin analogs that are used in the method of the present invention have, i.e. exhibit, less than 20% of the binding affinity to the corresponding insulin receptor compared to its parent insulin. In another embodiment, the insulin analogs provided herein have less than 10% of the binding affinity to the corresponding insulin receptor compared to its parent insulin. In another embodiment, the insulin analogs provided herein have less than 5% of the binding affinity to the corresponding insulin receptor compared to its parent insulin, such as less than 3% of the binding affinity compared to its parent insulin. For example, the insulin analogs provided herein may have between 0.1% to 10%, such as between 0.3% to 5% of the of the binding affinity to the corresponding insulin receptor compared to its parent insulin. Also, the insulin analogs provided herein may have between 0.5% to 3%, such as between 0.5% to 2% of the of the binding affinity to the corresponding insulin receptor compared to its parent insulin.

Methods for determining the binding affinity of an insulin analog to an insulin receptor are well known in the art. For example, the insulin receptor binding affinity can be determined by a scintillation proximity assay which is based on the assessment of competitive binding between [125I]-labelled parent insulin, such as [125I]-labelled human insulin, and the (unlabeled) insulin analog to the insulin receptor. The insulin receptor can be present in a membrane of a cell, e.g. of CHO (Chinese Hamster Ovary) cell, which overexpresses a recombinant insulin receptor. In an embodiment, the insulin receptor binding affinity is determined as described in the Examples section.

Binding of a naturally occurring insulin or an insulin analog to the insulin receptor activates the insulin signaling pathway. The insulin receptor has tyrosine kinase activity. Binding of insulin to its receptor induces a conformational change that stimulates the autophosphorylation of the receptor on tyrosine residues. The autophosphorylation of the insulin receptor stimulates the receptor's tyrosine kinase activity toward intracellular substrates involved in the transduction of the signal. The autophosphorylation of the insulin receptor by an insulin analog is therefore considered as a measure for signal transduction caused by said analog.

Thus, the insulin analogs that can be used in the method of the present invention may have a low binding activity, and consequently a lower receptor-mediated clearance rate, but are nevertheless capable of causing a relatively high signal transduction. Therefore, the insulin analogs provided herein could be used as long-acting insulins. In some embodiments, the insulin analog provided herein are capable of inducing 1 to 10%, such as 2 to 8%, insulin receptor autophosphorylation relative to the parent insulin (such as human insulin). Further, in some embodiments, the insulin analogs provided herein are capable of inducing 3 to 7%, such as 5 to 7% insulin receptor autophosphorylation relative to the parent insulin (such as human insulin). The insulin receptor autophosphorylation relative to a parent insulin can be determined as described in the Examples section.

As disclosed above, the activated sulfonamide of Formula (I) is covalently bond to the polypeptide in that the terminal carboxy group carrying the R^{x} group in the non-coupled state of the activated sulfonamide of Formula (I) is covalently bound to a suitable functional group of the polypeptide, for example to an amino group or a hydroxyl group of the polypeptide. According to at least one embodiment of the method, an amino group of the polypeptide to which the activated sulfonamide of formula (I) is covalently bound is an epsilon amino group of a lysine present at position B26 to B29, for example B29, of the B chain of human insulin, human insulin analog, precursor of human insulin or precursor of human insulin analog, for example of human insulin analog or a precursor of human insulin analog. In some embodiments, the polypeptide and the activated sulfonamide of formula (I) are connected by an amide bond, formed between the terminal carboxy group carrying the R^{x} group in the pre-coupled state of the activated sulfonamide of formula (I) and an amino group of the polypeptide, for example the epsilon amino group of a lysine present at position B26 to B29, for example B29, of the B chain of human insulin, human insulin analog, precursor of human insulin or precursor of human insulin analog, for example of human insulin analog or precursor of human insulin analog. It goes without saying that in case of an amide bond, the carboxyl group carrying the R^{x} group in the pre-coupled state is present in the conjugate formed as carbonyl group —C(═O)—, i.e. the amide bond is —C(═O)—NH— as shown below, wherein all residues E, A, R¹, R², X, as well as the indices m, s, p, n, t, r and q have the meaning as indicated above for formula (I) and the NH— group is already the part remaining from the peptide's amino group:

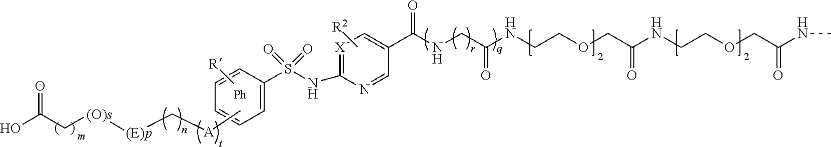

In an exemplary embodiment of the first aspect, a method of forming a conjugate of a sulfonamide and a polypeptide is provided, the method comprising:
a) Providing an activated sulfonamide, wherein the activated sulfonamide corresponds to Formula (I):

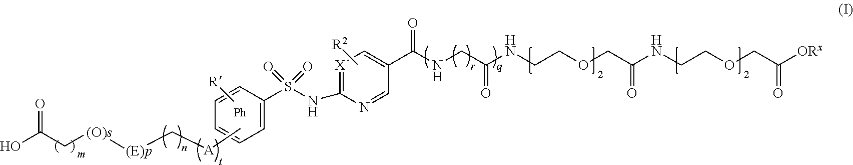

wherein
wherein
A is selected from the group consisting of oxygen atom, —CH₂CH₂— group, —OCH₂— group and —CH₂O— group;
E represents a —C₆H₃R— group with R being a hydrogen atom or a halogen atom, wherein the halogen atom is selected from the group consisting of fluorine, chlorine, bromine and iodine atom;
X represents a nitrogen atom or a —CH— group;
m is an integer in the range from 5 to 17;
n is zero or an integer in the range from 1 to 3;
p is zero or 1;
q is zero or 1;
r is an integer in the range from 1 to 6;
s is zero or 1;
t is zero or 1;
R¹ represents at least one residue selected from the group of hydrogen atom, halogen atom, C1 to C3 alkyl group and halogenated C1 to C3 alkyl group;
R² represents at least one residue selected from the group of hydrogen atom, halogen atom, C1 to C3 alkyl group and halogenated C1 to C3 alkyl group;
R^{x} represents an activation group;
wherein the combination of s being 1, p being zero, n being zero, A being an oxygen atom and t being 1 is excluded for Formula (I);
b) Providing an aqueous solution of a polypeptide having a free amino group, wherein the aqueous solution optionally comprises an alcohol;
c) Contacting the aqueous solution of b) with the activated sulfonamide of a); and
d) Reacting the activated sulfonamide with the polypeptide having a free amino group, obtaining a solution comprising the conjugate of a sulfonamide and a polypeptide, wherein the sulfonamide is covalently bonded to the polypeptide,
   wherein the polypeptide is an insulin polypeptide.

In some embodiments, the activated sulfonamide is an activated albumin binder, for example, if the method is a method of forming a conjugate of a sulfonamide and an insulin polypeptide.

Regarding the activated sulfonamide of formula (I): In some embodiments, s is zero, wherein the remaining residues and indices have the meaning as indicated above for formula (I).

According to at least one embodiment of the method, the polypeptide having a free amino group is a mature polypeptide or a precursor thereof, each having a free amino group, wherein the precursor of the mature polypeptide comprises an additional sequence of one or more further amino acid residues compared to the mature polypeptide. In some embodiments, “insulin polypeptide” is a mature insulin or a precursor of a mature insulin, wherein the precursor of the mature insulin comprises an additional sequence of one or more further amino acid residues compared to the mature insulin. The term “mature insulin” as referred to herein includes parent insulin, such as human insulin, and insulin analogs. In some embodiments, the mature insulin is an insulin analog, such as an insulin analog listed in Table 1 of the Examples section. For example, the insulin analog may be insulin analog 24 of Table 1.

In at least one embodiment of the method, the aqueous solution provided in a) has a pH value in the range of from 9 to 12, or in the range of from 9.5 to 11.5, or in the range of from to 11, wherein the pH value is determined with a pH sensitive glass electrode according to ASTM E 70:2007. In some embodiments, the pH value is adjusted in the respective range by addition of a base, such as a base selected from the group consisting of alkali hydroxides (lithium hydroxide, sodium hydroxide, potassium hydroxide), alkyl amines and mixtures of two or more thereof. In some embodiments, the base is selected from the group of tertiary alkyl amines N(C1-C5 alkyl)₃, primary alkyl amines H₂N—C(C1-C5 alkyl)₃ and mixtures of two or more thereof, wherein the C1-C5 alkyl groups of the tertiary amines and of the primary amines are each independently selected from branched or straight C1-C5 alkyl groups and wherein each C1-C5 alkyl group has at least one substituent selected from the group of hydrogen atom, hydroxyl group and carboxyl group. In some embodiments, the base is selected from the group of tertiary alkyl amines N(C1-C3 alkyl)₃, primary alkyl amines H₂N—C(C1-C3 alkyl)₃ and mixtures of two or more thereof, wherein the C1-C3 alkyl groups of the tertiary amines and of the primary amines are each independently selected from branched or straight C1-C3 alkyl groups and wherein each C1-C3 alkyl group has at least one substituent selected from the group of hydrogen atom, hydroxyl group and carboxyl group. In some embodiments, the base is selected from the group of bicine, trimethylamine, triethylamine, tris(hydroxymethyl)aminomethane and mixtures of two or more thereof. In some embodiments, the base comprises at least triethylamine.

In at least one embodiment of the method, contacting the aqueous solution of b) with the activated sulfonamide of a) according to step c) is done in that the activated sulfonamide of a) is added as a solution of the activated sulfonamide to the aqueous solution of b). In some embodiments, the solution of the activated sulfonamide is an organic solution, such as a solution comprising the activated sulfonamide and a polar aprotic organic solvent. In some embodiments, the polar aprotic organic solvent has an octanol-water-partition coefficient (K_{OW}) in the range of from 1 to 5 at standard conditions (T: 20-25° C., p: 1013 mbar). In some embodiments, the polar aprotic organic solvent has an octanol-water-partition coefficient (K_{OW}) in the range of from 2 to 4 at standard conditions (T: 20-25° C., p: 1013 mbar). In some embodiments, the polar aprotic organic solvent is selected from the group consisting of tetrahydrofuran, acetonitrile, dimethylformamide, and mixtures of two or more thereof. In some embodiments, the polar aprotic organic solvent is selected from the group of tetrahydrofuran, acetonitrile and mixtures of tetrahydrofuran and acetonitrile.

In at least one embodiment of the method, contacting the aqueous solution of b) with the activated sulfonamide of a) according to step c) is done in that the activated sulfonamide of a) is added in solid form to the aqueous solution of b). n some embodiments, the activated sulfonamide of a) is added in at least partially in crystalline form. In some embodiments, the activated sulfonamide of a) is added so that at least 90 weight-% thereof are in crystalline form.

In at least one embodiment of the method, step d) comprises:
d.1) Reacting the activated sulfonamide with a precursor of a mature polypeptide having a free amino group at a pH in the range from 9 to 12, obtaining a pre-conjugate comprising the sulfonamide and the precursor of the mature polypeptide, wherein the sulfonamide is covalently bonded to the precursor of the mature polypeptide by an amide bond C(═O)—NH— formed between the —C(═O)—O(R) of the (activated) sulfonamide of Formula (I) and the amino group of the precursor of the mature polypeptide;
d.2) Enzymatic digestion of the precursor of the mature insulin of the pre-conjugate obtained according to d.1), obtaining a solution comprising the conjugate of the sulfonamide and the mature polypeptide.

In some embodiments, reacting the activated sulfonamide with a precursor of the mature polypeptide having a free amino group according to d.1) is done at a pH in the range from 9.5 to 11.5. In some embodiments, reacting the activated sulfonamide with a precursor of the mature polypeptide having a free amino group according to d.1) is done at a pH in the range from 10 to 11. In some embodiments, the enzymatic digestion according to d.2) is done at a pH in the range below 9. In some embodiments, the enzymatic digestion according to d.2) is done at a pH in the range of 7 to 9.

In at least one embodiment, the method further comprises:
e) Isolating the conjugate of the sulfonamide and the mature polypeptide from the solution obtained in d) or d.2).

According to at least one embodiment, the activated sulfonamide has the formula (I-1)

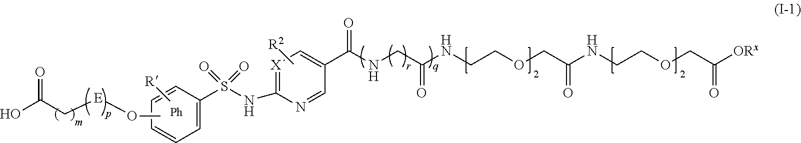

wherein:
E represents a —C₆H₃R— group with R being a hydrogen atom or a halogen atom, wherein the halogen atom is selected from the group consisting of fluorine, chlorine, bromine and iodine atom and is for example a fluorine atom;
X represents a nitrogen atom or a —CH— group;
p is zero or 1;
q is zero or 1;
r is an integer in the range from 1 to 6;
R¹ represents at least one residue selected from the group of hydrogen atom and halogen atom, wherein the halogen atom is for example a fluorine or chlorine atom;
R² represents at least one residue selected from the group of hydrogen atom, C1 to C3 alkyl group and halogenated C1 to C3 alkyl group, wherein the C1 to C3 alkyl group is for example a methyl group and the halogenated C1 to C3 alkyl group is for example perhalogenated such as a trifluoromethyl group;
R^{x} represents an activation group;
with m being an integer in the range from 5 to 15 if p is zero, or m being an integer in the range from 7 to 15 if p is 1.

In some embodiments, the residues R¹ and R² of the activated sulfonamide are hydrogen atoms. In some embodiments, the residue X of the activated sulfonamide represents a nitrogen atom. In some embodiments, the HOOC—(CH₂)ₘ—(O)ₛ-(E)ₚ-(CH₂)ₙ-(A)ₜ- group of formula (I) or the HOOC—(CH₂)ₘ-(E)ₚ-O— group of formula (I-1) of the activated sulfonamide is situated in meta or para position on phenyl ring Ph with respect to the —S(O)₂— group. In some embodiments, if p is 1, the HOOC—(CH₂)ₘ—(O)ₛ— group and the —(CH₂)ₙ-(A)ₜ- group are situated in meta or para position on (E)ₚ of formula (I) of the activated sulfonamide or the HOOC—(CH₂)ₘ— group and the —O— are situated in meta or para position on (E)ₚ of formula (I-1). In some embodiments, the index q of the activated sulfonamide is zero.

In some embodiments, the activated sulfonamide has the formula (I-1-2)

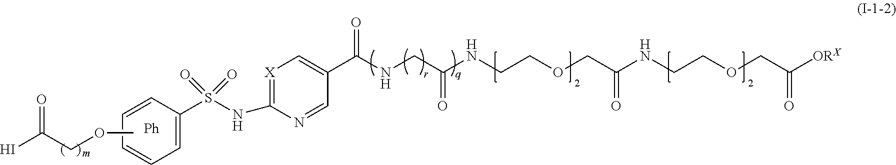

wherein X is a nitrogen atom or a —CH— group, for example a nitrogen atom; m is an integer in the range from 5 to 15; r is an integer in the range from 1 to 6; q is zero or 1, for example zero; R^{x} is an activation group; and the HOOC—(CH₂)ₘ—O— group is situated in meta or para position on phenyl ring Ph with respect to the —S(O)₂— group.

According to some embodiments, the activated sulfonamide has the formula (I-1-2a)

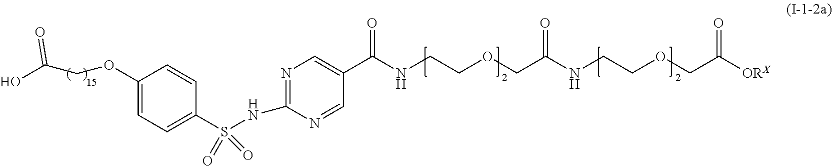

wherein R^{x} is an activation group.

In at least one embodiment of the method, the activation group R^{x} of the activated sulfonamide of Formula (I) is selected from the group consisting of 7-azabenzotriazole, 4-nitro benzene and N-succinimidyl-group. The 7-azabenzotriazole may be derived from HATU (1-[bis(dimethylamino)methylene]-1H-1,2,3-triazolo[4,5-b]pyridinium 3-oxide hexafluorophosphate) or HBTU (3-[bis(dimethylamino)methyliumyl]-3H-benzotriazol-1-oxide hexafluorophosphate). In some embodiments, R^{x} is a N-succinimidyl-group.

In at least one embodiment of the method, the aqueous solution of the polypeptide having a free amino group according to b) comprises an alcohol which is selected from the group consisting of C1-C4 monoalcohols and mixtures of two or more thereof. In some embodiments, the aqueous solution of the polypeptide having a free amino group according to b) comprises an alcohol which is selected from the group consisting of methanol, ethanol, propan-2-ol, propan-1-ol, butan-1-ol and mixtures of two or more thereof. In some embodiments, the aqueous solution of the polypeptide having a free amino group according to b) comprises an alcohol which is selected from the group consisting of ethanol, propan-2-ol, propan-1-ol, and mixtures of two or more thereof.

In at least one embodiment of the method, the aqueous solution according to b) comprises an alcohol, wherein the alcohol is present in the aqueous solution in an amount in the range from 0.0001 to 35 volume-%, based on the total volume of water and alcohol. In some embodiments, the aqueous solution according to b) comprises an alcohol, wherein the alcohol is present in the aqueous solution in an amount in the range from 0.001 to 30 volume-%, based on the total volume of water and alcohol. In some embodiments, the aqueous solution according to b) comprises an alcohol, wherein the alcohol is present in the aqueous solution in an amount in the range from 0.01 to 25 volume-%, based on the total volume of water and alcohol. In some embodiments, the aqueous solution according to b) comprises an alcohol, wherein the alcohol is present in the aqueous solution in an amount in the range from 0.1 to 20 volume-%, based on the total volume of water and alcohol.

In at least one embodiment of the method, the enzymatic digestion according to d.2) comprises use of at least one enzyme selected from the group consisting of trypsin, a TEV protease (Tobacco Etch Virus protease) and mixtures of two or more thereof.

In at least one embodiment of the method, the mature polypeptide is a mature insulin, which comprises an A chain and a B chain, wherein the A chain comprises at least one mutation relative to the A chain of human insulin and/or the B chain comprises at least mutation relative to human insulin. In some embodiments, the at least one mutation relative to the A chain of human insulin is a substitution at position A14, such as a substitution with an amino acid selected from the group consisting of glutamic acid (Glu), aspartic acid (Asp) and histidine (His), and/or a substitution at position A21, such as a substitution with glycine (Gly). In some embodiments, the mutation relative to the B chain of human insulin is a substitution at position B16, such as a substitution with an amino acid selected from the group consisting of valine (Val), isoleucine (Ile), leucine (Leu), alanine (Ala) or histidine (His), a substitution at position B25, such as a substitution with valine (Val), isoleucine (Ile), leucine (Leu), alanine (Ala) or histidine (His), and/or a deletion at position B30.

In some embodiments, the insulin analog comprises a mutation at position B16 which is substituted with a hydrophobic amino acid. Thus, the amino acid at position B16 (tyrosine in human, bovine and porcine insulin) is replaced with a hydrophobic amino acid.

In another embodiment, the insulin analog comprises a mutation at position B25 which is substituted with a hydrophobic amino acid. Thus, the amino acid at position B25 (phenylalanine in human, bovine and porcine insulin) is replaced with a hydrophobic amino acid.

In another embodiment, insulin analog comprises a mutation at position B16 which is substituted with a hydrophobic amino acid and a mutation at position B25 which is substituted with a hydrophobic amino acid. The hydrophobic amino acid may be any hydrophobic amino acid. For example, the hydrophobic amino acid may be an aliphatic amino acid such as a branched-chain amino acid.

In some embodiments, the hydrophobic amino acid used for the substitution at position B16 and/or B25 is isoleucine, valine, leucine, alanine, tryptophan, methionine, proline, glycine, phenylalanine or tyrosine. In some embodiments, the hydrophobic amino acid used for the substitution at position B16 and/or B25 is isoleucine, valine, leucine, such as valine. Further, it is envisaged that the amino acid at position B16 and/or at position B25 is substituted with a histidine.

The insulin analog may comprise further mutations. For example, the insulin analog may further comprise a mutation at position A14. Such mutations are known to increase protease stability (see e.g. WO 2008/034881 A1 [Novo Nordisk, Nielsen]). In some embodiments, the amino acid at position A14 is substituted with glutamic acid (Glu). In some embodiments, the amino acid at position A14 is substituted with aspartic acid (Asp). In some embodiments, the amino acid at position A14 is substituted with histidine (His).

Further, the insulin analog may comprise a mutation at position B30. In some embodiment, the mutation at position B30 is the deletion of threonine at position B30 of the parent insulin (also referred to as Des(B30)-mutation).

Further, the insulin analog of the present invention may further comprise a mutation at position B3 which is substituted with a glutamic acid (Glu), and/or a mutation at position A21 which is substituted with glycine (Gly).

In some embodiments, the insulin analog comprises a substitution at position A14, a substitution a position B25, and a deletion at position B30 (i.e. the amino acid at position B30 is absent).

In some embodiments, the A chain of the insulin analog comprises or consists of the following sequence:

(SEQ ID NO: 109)
GIVEQCCTSICSL Xaa9 QLENYCN,

and the B-chain of the insulin analog comprises or consists of the following sequence:

(SEQ ID NO: 110)
FVNQHLCGSHLVEAL Xaa10 LVCGERGF Xaa11 YTPK,

wherein Xaa9 is glutamic acid (Glu), aspartic acid (Asp) or histidine (His)
wherein Xaa10 is tyrosine (Tyr), valine (Val), isoleucine (Ile), leucine (Leu), alanine (Ala) or histidine (His), and/or
wherein Xaa11 is phenylalanine (Phe), valine (Val), isoleucine (Ile), leucine (Leu), alanine (Ala) or histidine (His).

In some embodiments, Xaa9 is glutamic acid (Glu), Xaa10 is tyrosine (Tyr), and Xaa11 is valine (Val), isoleucine (Ile), or leucine (Leu). In some embodiments, Xaa9 is glutamic acid

(Glu), Xaa10 is tyrosine (Tyr), and Xaa11 is valine (Val).

In some embodiments, the mature insulin is selected from the group consisting of
Leu(B16)-human insulin,
Val(B16)-human insulin,
Ile(B16)-human insulin,
Leu(B16)Des(B30)-human insulin,
Val(B16)Des(B30)-human insulin,
Ile(B16)Des(B30)-human insulin,
Leu(B25)-human insulin,
Val(B25)-human insulin,
Ile(B25)-human insulin,
Leu(B25)Des(B30)-human insulin,
Val(B25)Des(B30)-human insulin,
Ile(B25)Des(B30)-human insulin,
Glu(A14)Leu(B16)Des(B30)-human insulin,
Glu(A14)Ile(B16)Des(B30)-human insulin,
Glu(A14)Val(B16)Des(B30)-human insulin,
Glu(A14)Leu(B16)-human insulin,
Glu(A14)Ile(B16)-human insulin,
Glu(A14)Val(B16)-human insulin,
Glu(A14)Leu(B25)Des(B30)-human insulin,
Glu(A14)Ile(B25)Des(B30)-human insulin,
Glu(A14)Val(B25)Des(B30)-human insulin,
Glu(A14)Leu(B25)-human insulin,
Glu(A14)Ile(B25)-human insulin,
Glu(A14)Val(B25)-human insulin,
Glu(A14)Gly(A21)Glu(B3)Val(B25)Des(B30)-human insulin,
Glu(A14)Ile(B16)Ile(B25)Des(B30)-human insulin,
Glu(A14)Glu(B3)Ile(B16)Ile(B25)Des(B30)-human insulin,
Glu(A14)Ile(B16)Val(B25)Des(B30)-human insulin,
Glu(A14)Gly(A21)Glu(B3)Ile(B16)Val(B25)Des(B30)-human insulin,
Glu(A14)Val(B16)Ile(B25)Des(B30)-human insulin,
Glu(A14)Val(B16)Val(B25)Des(B30)-human insulin,
Glu(A14)Glu(B3)Val(B16)Val(B25)Des(B30)-human insulin,
Glu(A14)Gly(A21)Glu(B3)Val(B16)Val(B25)Des(B30)-human insulin,
Glu(A14)Gly(A21)Glu(B3)Val(B25)-human insulin,
Glu(A14)Ile(B16)Ile(B25)-human insulin,
Glu(A14)Glu(B3)Ile(B16)Ile(B25)-human insulin,
Glu(A14)Ile(B16)Val(B25)-human insulin,
Glu(A14)Gly(A21)Glu(B3)Ile(B16)Val(B25)-human insulin,
Glu(A14)Val(B16)Ile(B25)-human insulin,
Glu(A14)Val(B16)Val(B25)-human insulin,
Glu(A14)Glu(B3)Val(B16)Val(B25)-human insulin, and
Glu(A14)Gly(A21)Glu(B3)Val(B16)Val(B25)-human insulin
Glu (A14)His(B25)Des(B30) human insulin, and
Glu (A14)His(B16)His(B25) Des(B30) human insulin.

In some embodiments, the amino acid residues referred to herein are L-amino acid residues (such as L-isoleucine, L-valine, or L-leucine). Accordingly, the amino acid residues (or the derivatives thereof) used for e.g. the substitution at position B16, B25, A14 and/or A21 are typically L-amino acid residues.

In another embodiment, the insulin analog is Leu(B25)Des(B30)-Insulin (such as Leu(B25)Des(B30)-human insulin). The sequence of this analog is, e.g., shown in Table 1 of the Examples section (see Analog 11).

In another embodiment, the insulin analog is Val(B25)Des(B30)-Insulin (such as Val(B25)Des(B30)-human insulin). The sequence of this analog is, e.g., shown in Table 1 of the Examples section (see Analog 12).

In another embodiment, the insulin analog is Glu(A14)Ile(B25)Des(B30)-Insulin (such as Glu(A14)Ile(B25)Des(B30)-human insulin). The sequence of this analog is, e.g., shown in Table 1 of the Examples section (see Analog 22).

In another embodiment, the insulin analog is Glu(A14)Val(B25)Des(B30)-Insulin (such as Glu(A14)Val(B25)Des(B30)-human insulin). The sequence of this analog is, e.g., shown in Table 1 of the Examples section (see Analog 24).

In another embodiment, the insulin analog is Glu(A14)Gly(A21)Glu(B3)Val(B25)Des(B30)-Insulin (such as Glu(A14)Gly(A21)Glu(B3) Val(B25)Des(B30)-human insulin). The sequence of this analog is, e.g., shown in Table 1 of the Examples section (see Analog 25).

In another embodiment, the insulin analog is Glu(A14)Ile(B16)Ile(B25)Des(B30)-Insulin(such as Glu(A14)Ile(B16)Ile(B25)Des(B30)-human insulin). The sequence of this analog is, e.g., shown in Table 1 of the Examples section (see Analog 29).

In another embodiment, the insulin analog is Glu(A14)Glu(B3)Ile(B16)Ile(B25)Des(B30)-Insulin(such as Glu(A14)Glu(B3)Ile(B16) Ile(B25)Des(B30)-human insulin). The sequence of this analog is, e.g., shown in Table 1 of the Examples section (see Analog 30). (FVEQHLCGSHLVEALILVCGERGFIYTPK).

In another embodiment, the insulin analog is Glu(A14)Ile(B16)Val(B25)Des(B30)-Insulin (such as Glu(A14)Ile(B16)Val(B25)Des(B30)-human insulin) The sequence of this analog is, e.g., shown in Table 1 of the Examples section (see Analog 32).

In another embodiment, the insulin analog is Glu(A14)Gly(A21)Glu(B3) Ile(B16)Val(B25)Des(B30)-Insulin (such as Glu(A14)Gly(A21)Glu(B3)Ile(B16)Val(B25) Des(B30)-human insulin). The sequence of this analog is, e.g., shown in Table 1 of the Examples section (see Analog 33).

In another embodiment, the insulin analog is Glu(A14)Val(B16)Ile(B25)Des(B30)-Insulin (such as Glu(A14)Val(B16)Ile(B25)Des(B30)-human insulin). The sequence of this analog is, e.g., shown in Table 1 of the Examples section (see Analog 35).

In another embodiment, the insulin analog is Glu(A14)Val(B16)Val(B25)Des(B30)-Insulin (such as Glu(A14)Val(B16)Val(B25) Des(B30)-human insulin). The sequence of this analog is, e.g., shown in Table 1 of the Examples section (see Analog 38).

In another embodiment, the insulin analog is Glu(A14)Glu(B3)Val(B16)Val(B25)Des(B30)-Insulin (such as Glu(A14)Glu(B3)Val(B16) Val(B25)Des(B30)-human insulin). The sequence of this analog is, e.g., shown in Table 1 of the Examples section (see Analog 39).

In another embodiment, the insulin analog is Glu(A14)Gly(A21)Glu(B3)Val(B16)Val(B25)Des(B30)-Insulin (such as Glu(A14)Gly(A21) Glu(B3)Val(B16)Val(B25)Des(B30)-human insulin). The sequence of this analog is, e.g., shown in Table 1 of the Examples section (see Analog 40).

In another embodiment, the insulin analog is Glu(A14)His(B25)Des(B30) human insulin.

In another embodiment, the insulin analog is Glu(A14)His(B16)His(B25) Des(B30) human insulin.

In at least one embodiment of the method, the precursor of the mature polypeptide is a precursor of a mature insulin, which comprises a sequence as listed in detail herein above for the mature insulin, comprising an A chain and a B chain, and an additional linker peptide, which has a length of at least two amino acid residues. Optionally, the linker peptide has a length in the range from 2 to 30 amino acid residues, for example a length in the range from 4 to 9 amino acid residues. In some embodiments, the precursor is a precursor as defined in section B of the present application.

In at least one embodiment of the method, the first amino acid of the linker peptide is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or a valine residue, for example, the first amino acid of the linker peptide is selected from alanine, arginine, asparagine, aspartic acid, glutamine, glutamic acid, glycine, histidine, isoleucine, leucine, methionine, phenylalanine, proline, serine, threonine, tryptophan, tyrosine, or valine. In some embodiments, the first amino acid of the linker peptide is a threonine residue, phenylalanine residue, a glutamine residue, a glutamic acid residue, an asparagine residue or an aspartic acid residue. At least these amino acid residues for the first amino acid of the linker peptide are amino acid residues having an amino group at the N-terminus which has a low nucleophilicity. This reduces the reactivity regarding a reaction with the activated carboxyl group —COOR^{x} of the sulfonamide.

In at least one embodiment of the method, the last amino acid of the linker peptide is an arginine residue.

In at least one embodiment of the method, the linker peptide comprises or consists of the following sequence

(SEQ ID NO: 106)
Xaa1-Xaa2-Xaa3-Xaa4-Xaa5-Xaa6-Xaa7-Xaa8-Arg

wherein Xaa1 to Xaa8 may be as follows:

Xaa1 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. In some embodiments, Xaa1 is threonine, phenylalanine, glutamine, glutamic acid, asparagine or aspartic acid.

Xaa2 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, or valine. In some embodiments, Xaa2 is glutamic acid. Alternatively, Xaa2 is absent.

Xaa3 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. In some embodiments, Xaa3 is glycine. Alternatively, Xaa3 is absent.

Xaa4 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa4 is absent.

Xaa5 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa5 is absent.

Xaa6 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa6 is absent.

Xaa7 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa7 is absent.

Xaa8 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa8 is absent.

In at least one embodiment of the method, the linker peptide has the sequence TEGR (SEQ ID NO: 112). A pre-conjugate comprising an exemplary sulfonamide and a precursor of a mature polypeptide, wherein the linker peptide has the sequence TEGR (SEQ ID NO: 112), and an exemplary sulfonamide is shown in FIG. 8.

In at least one embodiment of the method, the linker peptide is a linker peptide as defined in section B of the present application.

In at least one embodiment of the method, the sulfonamide is covalently bonded to the mature polypeptide and the precursor thereof respectively by an amide bond C(═O)—NH— formed between the —C(═O)—O(R^{x}) of the (activated) sulfonamide of Formula (I) and the free amino group of the mature polypeptide and the precursor thereof respectively. In some embodiments, the free amino group of the polypeptide is the amino group of a lysine comprised in the mature polypeptide and the precursor thereof respectively. In some embodiments, the free amino group of the polypeptide is the amino group of a terminal lysine. In some embodiments, the free amino group of the polypeptide is the amino group of a lysine present at a C terminus of the mature polypeptide and the precursor thereof respectively. In some embodiments, the free amino group of the polypeptide is the amino group of a lysine present at the C terminus of the B-chain.

The (cleavable) linker peptide, especially the linker peptide TEGR (SEQ ID NO: 112), protects the N-terminus of the A-chain from coupling with the activated sulfonamide of Formula (I). The peptide TEGR (SEQ ID NO: 112) does not or only to a minor extend below 1% react with the activated carboxyl group —COOR^{x} of the sulfonamide, which lowers the excess of sulfonamide required. The cleavage of TEGR (SEQ ID NO: 112) after the coupling with the sulfonamide can be achieved in one pot by adjusting the pH to a value in the range below 9, followed by the addition of trypsin, a TEV protease (Tobacco Etch Virus protease) or a mixture of these two enzymes. In some embodiments, the pH is adjusted to a value in the range of 7 to 9. In some embodiments, the pH is adjusted to a value of approximately 8. Since the linker peptide, typically the TEGR (SEQ ID NO: 112) peptide, protects the A1 amino acid, i.e. its free NH₂-group, the A1-acylated byproduct is not produced. The separation of the desired compound is thus simplified, because the A1-acylated byproduct shows similar retention times as the most desired product, wherein only the lysine at B29 is coupled to the sulfonamide conjugate.

Exemplary conjugates of the sulfonamide of formula (I) and polypeptides, for example, insulin analogs, which are obtained or obtainable by the method described herein above are disclosed in section B; exemplary conjugates are shown in FIGS. 3 to 6.

In at least one embodiment of the method, the activated sulfonamide of Formula (I) is obtained or obtainable from a protected activated sulfonamide of Formula (0)

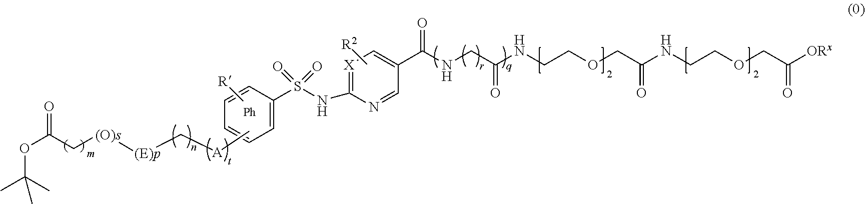

wherein A, E, X, m, n, p, q, r, s, t, R¹, R² and R^{x} have the meaning as defined herein above, wherein the protected activated sulfonamide of Formula (0) is optionally deprotected by addition of one or more acids, for example by addition of at least trifluoroacetic acid.

In a second aspect, a conjugate obtained or obtainable from the method of any one of embodiments as described herein above is provided.

In a third aspect, a precursor of a mature polypeptide comprising a sequence of a mature polypeptide according to the embodiment as described herein above and an additional linker peptide as defined in any one of embodiments as described herein above covalently bonded to the N terminus of the mature polypeptide A-chain is provided.

In a fourth aspect, a procedure for crystallizing an activated sulfonamide corresponding to Formula (I) is provided

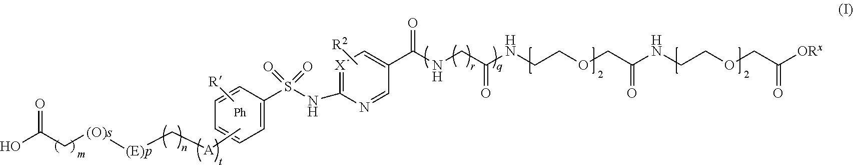

wherein A, E, X, m, n, p, q, r, s, t, R¹, R² and R^{x} have the meaning as defined above with respect to the method for preparing a conjugate, comprising
A) Providing a solution comprising the activated sulfonamide and an organic solvent;
B) Removing the organic solvent at least partially, for example by distillation, obtaining a phase of the activated sulfonamide having a reduced amount of the organic solvent compared to the solution provided in A);
C) Adding organic solvent to the phase obtained in B) obtaining a solution of the activated sulfonamide; and
D) Repeating step B) with the solution obtained in C) obtaining a phase of the activated sulfonamide having a reduced amount of the organic solvent compared to the solution obtained in C);
E) Optionally repeating steps C) and D) at least one further time.

The “phase of the activated sulfonamide having a reduced amount of the organic solvent compared to the solution provided in A)” comprises solutions (i.e. liquid phases) of the activated sulfonamide having a reduced amount of the organic solvent compared to the solution provided in A), oily phases of the activated sulfonamide and solid phases of the activated sulfonamide. In at least one embodiment, the procedure for crystallizing an activated sulfonamide corresponding to Formula (I) comprises an additional step:
F) Adding organic solvent to the phase of the activated sulfonamide obtained in D) and/or to the phase of the activated sulfonamide obtained in E) and keeping the resulting solution at a temperature in the range from 5 to 40° C. for at least one hour, thus obtaining a precipitate comprising the activated sulfonamide in solid form.

Said precipitate obtained according to (F) can be separated from the solution by means known in the art, for example, by filtration.

In some embodiments, the resulting solution in F) is kept at a temperature in the range from to 35° C. In some embodiments, the resulting solution in F) is kept at a temperature in the range from 20 to 30° C. In some embodiments, the resulting solution in F) is kept at the respective temperature for 1 to 72 hours. In some embodiments, the resulting solution in F) is kept at the respective temperature for 10 to 48 hours. In some embodiments, the resulting solution in F) is kept at the respective temperature for 15 to 30 hours. In some embodiments, the precipitate obtained in F) comprises the activated sulfonamide at least partially in crystalline form. In some embodiments, the precipitate obtained in F) comprises the activated sulfonamide at least 90 weight-% in crystalline form.

In at least one embodiment of the procedure for crystallizing an activated sulfonamide corresponding to Formula (I), the solution comprising the activated sulfonamide and an organic solvent the organic solvent provided in A) further comprises trifluoroacetic acid. In at least one embodiment, the organic solvent is selected from the group of organic solvents capable of forming an aceotropic mixture with trifluoroacetic acid. In at least one embodiment of the procedure for crystallizing an activated sulfonamide corresponding to Formula (I), the organic solvent is a polar aprotic organic solvent. In some embodiments, the polar aprotic organic solvent has an octanol-water-partition coefficient (K_{OW}) in the range from 1 to 5 at standard conditions (temperature: 20-25° C., pressure: 1013 mbar). In some embodiments, the polar aprotic organic solvent has an octanol-water-partition coefficient (K_{OW}) in the range from 2 to 4, at standard conditions (temperature: 20-25° C., pressure: 1013 mbar). In some embodiments, the organic solvent is selected from the group of acetonitrile, tetrahydrofuran and mixtures of acetonitrile and tetrahydrofuran. In some embodiments, the organic solvent at least comprises acetonitrile.

Due to, for example, synthesis reasons, the activated sulfonamide corresponding to Formula (I) comprises minor amounts of trifluoroacetic acid (less than 5 weight-% based on the weight of the activated sulfonamide). However, these minor amounts disturb the solidification and crystallisation respectively of the activated sulfonamide, which results in the activated sulfonamide being present in oil form. The repeated addition of organic solvent and the off-distillation thereof enables an aceotropic removal of trifluoroacetic acid, which in turn results in solidification/crystallisation of the activated sulfonamide after reduction and removal respectively of the trifluoroacetic acid amount. According to the Dortmunder Datenbank, acetonitrile has an octanol-water-partition coefficient (K_{OW}) of 2, tetrahydrofuran (THF) has a K_{OW} of 4, dimethylformamide has a K_{OW} of 4.

The organic solvent used for providing the solution in A), the organic solvent added in C), the optional organic solvent used in E) and the organic solvent used in F) are the same or different and are independently selected from the group of organic solvents capable of forming an aceotropic mixture with trifluoroacetic acid and/or from the group of polar aprotic organic solvents, which may have a K_{OW} in the range of from 1-5. In some embodiments, the polar aprotic organic solvents has a K_{OW} in the range from 2-4. In some embodiments, the same organic solvent is used for providing the solution in A), in C), optionally in E) and in F).

In a fifth aspect, a solid form of the activated sulfonamide corresponding to Formula (I) is provided

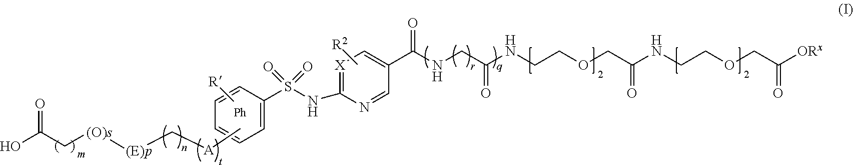

wherein A, E, X, m, n, p, q, r, s, t, R¹, R² and R^{x} have the meaning as defined herein above. In some embodiments, the activated binder is crystalline.

In one embodiment, an exemplary sulfonamide of Formula (I) is prepared by coupling of two building blocks A and B as shown in schema 1 below, wherein the coupling of building blocks A and B gives the exemplary sulfonamide, which is called Pyrimidine-bis-OEG-acid:

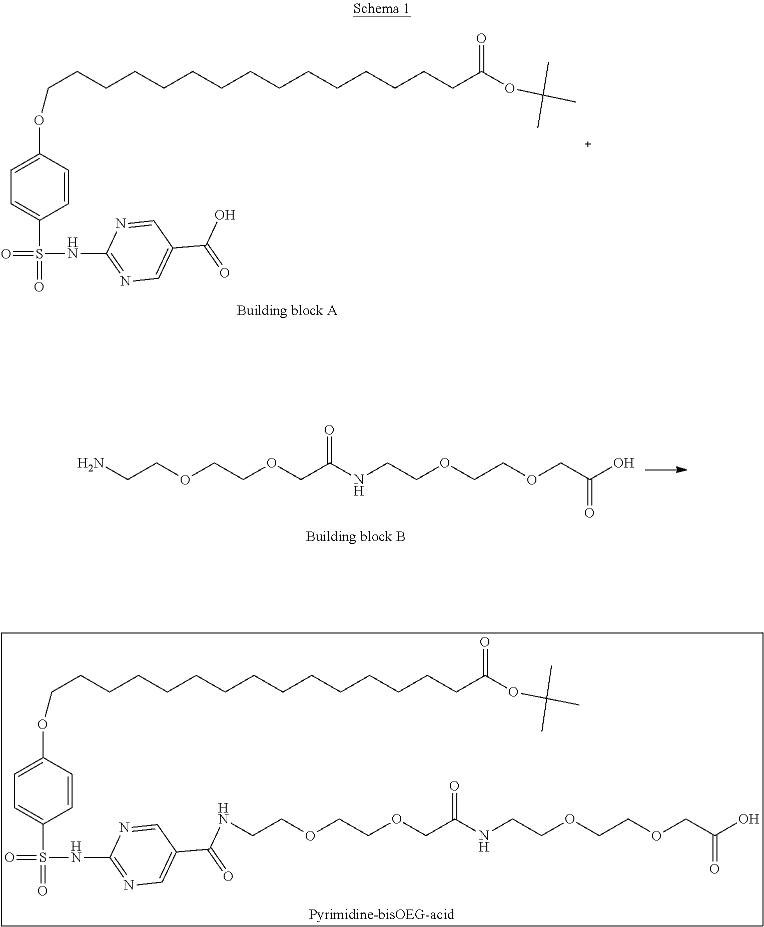 [SMILES: CC(C)(C)OC(=O)CCCCCCCCCCCCCCCOc1ccc(S(=O)(=O)Nc2ncc(C(=O)O)cn2)cc1 ; NCCOCCOCC(=O)NCCOCCOCC(=O)O ; CC(C)(C)OC(=O)CCCCCCCCCCCCCCCOc1ccc(S(=O)(=O)Nc2ncc(C(=O)NCCOCCOCC(=O)NCCOCCOCC(=O)O)cn2)cc1]

Schema 2 shows the synthesis of building block A, and schema 3 shows the synthesis of building block B:

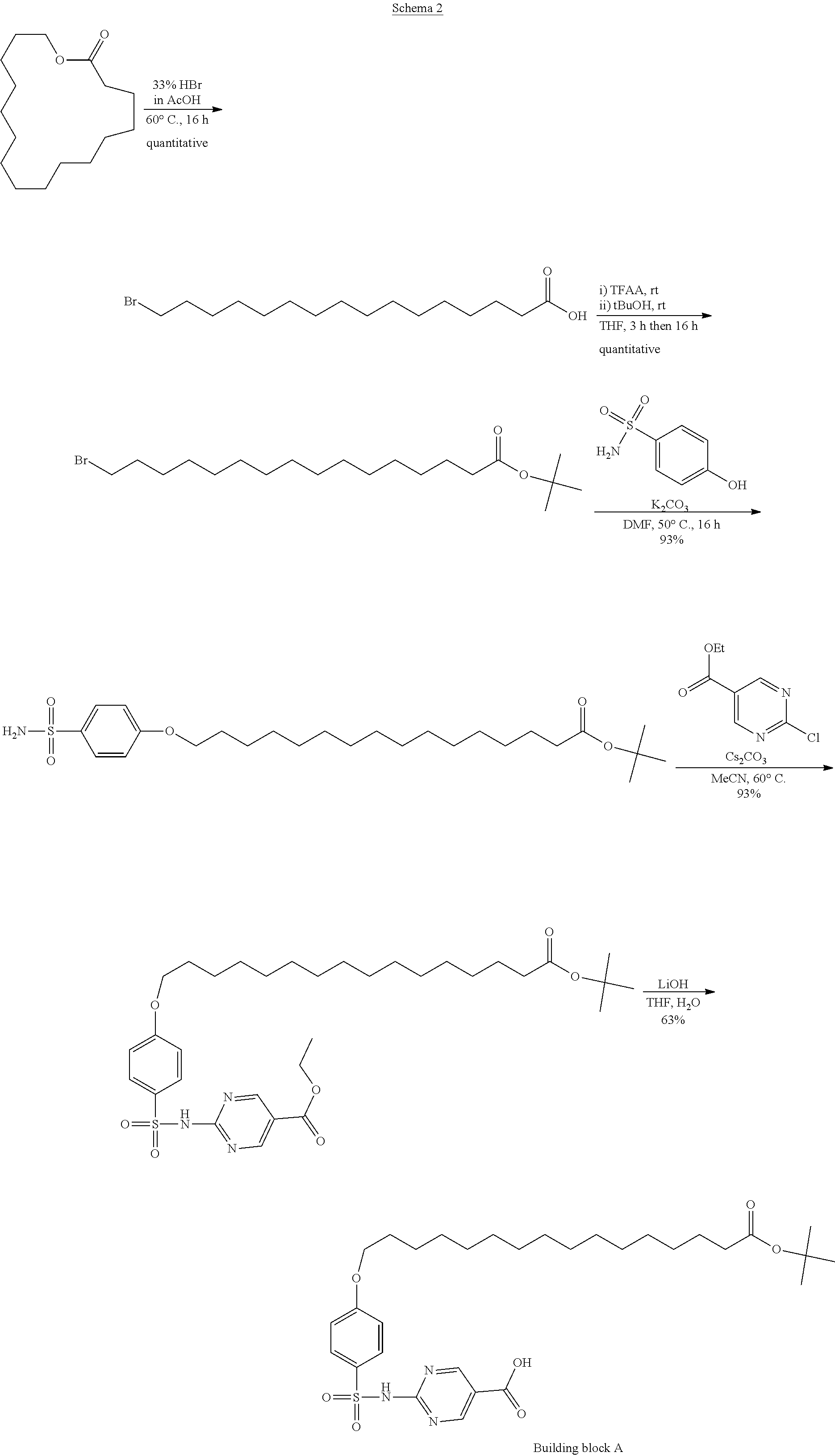

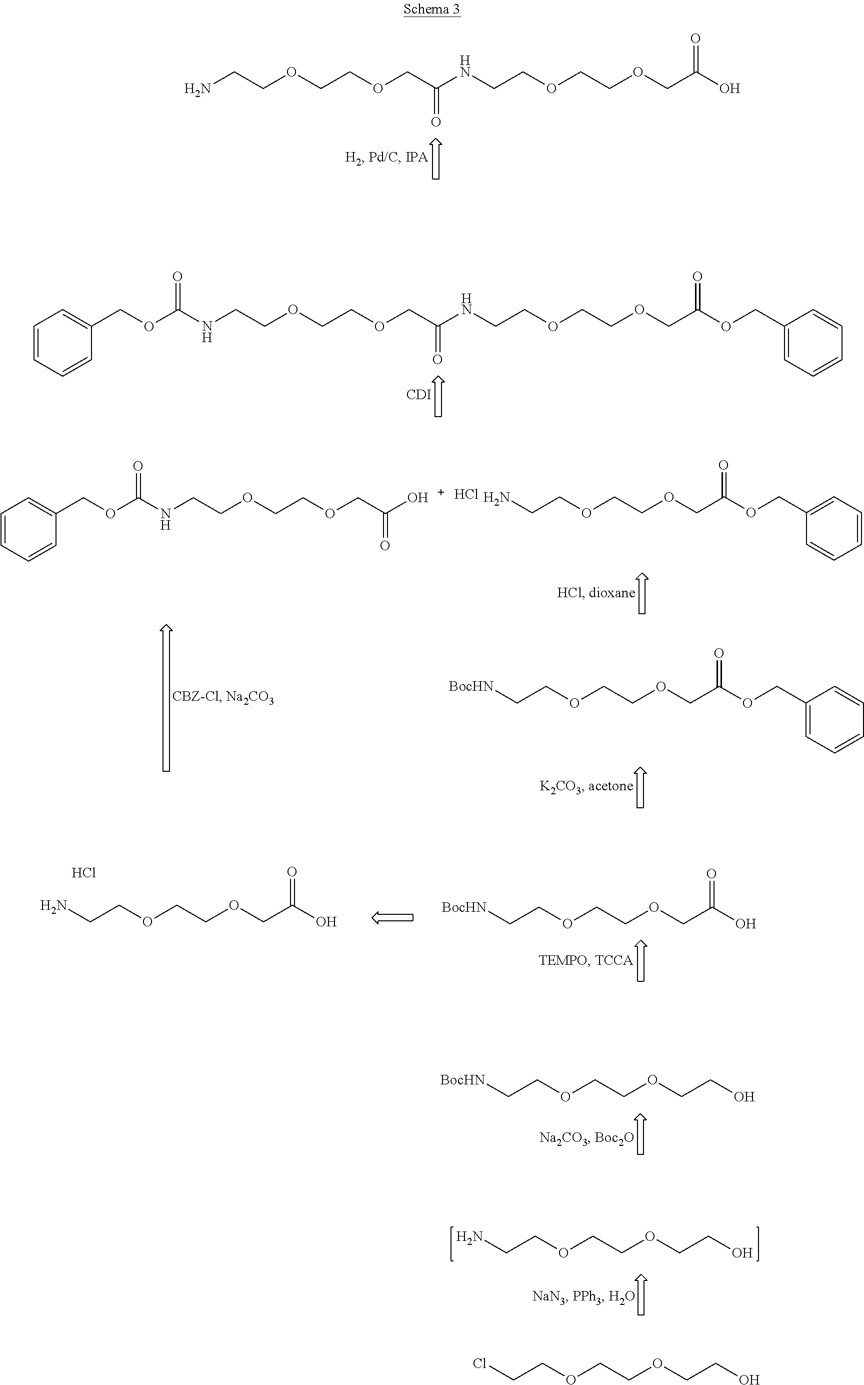

The definitions and explanations provided herein above in Section A shall apply mutatis mutandis to the embodiments described herein below in section B.

Section B

As set forth in section A, the presence of one or more additional amino acid residues at the N-terminus of the A-chain, for example the presence of a TEGR (SEQ ID NO: 112) peptide, protects the A1 amino acid of the A-chain, i.e. the free NH₂-group of the A-chain. Accordingly, the A1-acylated byproduct is not produced when preparing a conjugate of an albumin binder and an insulin precursor comprising an N-terminally extended A-chain and a B-chain. After conjugation, the one or more additional peptides at the N-terminus of the A-chain can be advantageously removed by proteolytic cleavage, e.g. with trypsin, thereby generating the conjugate of a mature insulin (such as an insulin analog) and an albumin binder. The separation of the desired conjugated is simplified, because the A1-acylated byproduct shows similar retention times as the desired product, wherein only the lysine at B29 is coupled to the albumin binder conjugate.

Further, the amount of binder used for the conjugation might be reduced, if the first amino acid of the N-terminally extended A-chain has a lower nucleophilicity than the A1 amino acid. This reduces the reactivity regarding a reaction with the activated carboxyl group —COOR^{x} of the albumin binder. For example, threonine has a lower nucleophilicity than glycine which is frequently found at the A1 position of insulin analogs.

An insulin precursor comprising an N-terminally extended A-chain and a B-chain can be generated by cleaving a proinsulin comprising from N- to C-terminus an insulin B-chain, a linker peptide and an insulin A-chain with a protease between the last amino acid of the insulin B-chain and the first amino acid of the linker peptide. The generated N-terminally extended A-chain comprises, from N- to C-terminus, the linker peptide and the A-chain. The linker peptide then protects the A1 amino acid of the A-chain in the subsequent conjugation step.

Accordingly, the present invention relates to a process for generating a conjugate of an albumin binder and a mature insulin, said process comprising
a) Providing a proinsulin comprising from N- to C-terminus an insulin B-chain, a linker peptide and an insulin A-chain,
b) Cleaving the proinsulin provided in step a) with a first protease between the last amino acid of the insulin B-chain and the first amino acid of the linker peptide, thereby generating an insulin precursor, said insulin precursor comprising the insulin B-chain and an N-terminally extended A-chain comprising the linker peptide and the A-chain,
c) Contacting said insulin precursor with an albumin binder, wherein the albumin binder comprises a functional group capable of binding to albumin;
   thereby generating a conjugate of an albumin binder and the insulin precursor,
d) Cleaving the N-terminally extended A-chain of said insulin precursor comprised by the conjugate with a second protease between the last amino acid of the linker peptide and the first amino acid of the A-chain, thereby generating a conjugate of an albumin binder and a mature insulin.

An “albumin binder” is a compound capable of binding non-covalently to an albumin, for example, human albumin, for example in a blood sample.

Optionally, the albumin binder is an activated albumin binder. The activated albumin binder may comprise an activated carboxyl group —COOR^{x}, wherein R^{x} is an activation group. The activation group R^{x} is in at least one embodiment selected from the group consisting of 7-azabenzotriazole, for example derived from HATU or HBTU, 4-nitro benzene and N-succinimidyl-group. Optionally, R^{x} is a N-succinimidyl-group.

In at least one embodiment, the albumin binder comprises a functional group capable of binding to albumin, such as human serum albumin. Optionally, the functional group capable of binding to albumin is a carboxyl group or a bioisostere of a carboxyl group. Optionally, the functional group capable of binding to albumin is selected from the group consisting of a carboxyl group, hydroxamic acid group, hydroxamic ester group, phosphonic acid group, phosphinic acid group, sulfonic acid group, sulfinic acid group, sulfonamide group, acyl sulfonamide group, sulfonyl urea group, acyl urea group, tetrazole group, thiazolinine dione group, oxazolindine dione group, oxadiazol-5(4H)-one group, thiadiazol-5(4H)-one group, oxathiadiazole-2-oxide group, oxadiazole-5(4H)-thione group, isoxazole group, tetramic acid group, cyclopentane 1,3-diones, cyclopentane 1,2-diones, squaric acid derivatives, substituted phenols, —CO-Asp, —CO-Glu, —CO-Gly, —CO-Sar (—CO-sarcosine), —CH(COOH)₂, and —N(CH₂COOH)₂. Optionally, the functional group capable of binding to albumin is selected from the group consisting of a carboxyl group, —CO-Asp, —CO-Glu, —CO-Gly, —CO-Sar, —CH(COOH)₂, —N(CH₂COOH)₂, sulfonic acid group (—S0₃H) and phosphonic acid group (—P0₃H).

Optionally, the albumin binder comprises an acyl moiety. For example, the acyl moiety has the general formula Acy-AA1n-AA2m-AA3p- (Formula N), wherein:
n is 0 or an integer in the range from 1 to 3; m is 0 or an integer in the range from 1 to 10; p is 0 or an integer in the range from 1 to 10;
Acy is a fatty acid or a fatty diacid comprising from about 8 to about 24 carbon atoms;
AA1 is a neutral linear or cyclic amino acid residue;
AA2 is an acidic amino acid residue;
AA3 is a neutral, alkylene glycol-containing amino acid residue.

In Formula N, the order by which AA1, AA2 and AA3 appear in the formula can be interchanged independently; AA2 can occur several times along the formula (e.g., Acy-AA2-AA3rAA2-); AA2 can occur independently (and a being different species) several times along the formula (e.g., Acy-AA2-AA3-AA2-). In Formula N, the bonds between Acy, AA1, AA2 and/or AA3 are amide (peptide) bonds.

Optionally, AA1 is selected from the group consisting of: Gly, D- or L-Ala, beta-Ala, 4-aminobutyric acid, 5-aminovaleric acid, 6-aminohexanoic acid, D- or L-Glu-alpha-amide, D- or L-Glu-gamma-amide, D- or L-Asp-alpha-amide, D- or L-Asp-beta-amide, 7-aminoheptanoic acid and 8-aminooctanoic acid.

Optionally, AA2 is selected from the group consisting of L- or D-Glu, L- or D-Asp, L- or D-homoGlu.

Optionally, the neutral cyclic amino acid residue designated AA1 is an amino acid containing a saturated 6-membered carbocyclic ring, optionally containing a nitrogen hetero atom, and the ring may be a cyclohexane ring or a piperidine ring. Optionally, the molecular weight of this neutral cyclic amino acid is in the range from about 100 to about 200 Da.

The acidic amino acid residue designated AA2 may be an amino acid with a molecular weight of up to about 200 Da comprising two carboxylic acid groups and one primary or secondary amino group. Alternatively, acidic amino acid residue designated AA2 is an amino acid with a molecular weight of up to about 250 Da comprising one carboxylic acid group and one primary or secondary sulfonamide group.

The neutral, alkylene glycol-containing amino acid residue designated AA3 is an alkylene glycol moiety, optionally an oligo- or polyalkylene glycol moiety containing a carboxylic acid functionality at one end and an amino group functionality at the other end. Herein, the term alkylene glycol moiety covers mono-alkylene glycol moieties as well as oligoalkylene glycol moieties. Mono- and oligoalkyleneglycols comprises mono- and oligoethyleneglycol based, mono- and oligopropyleneglycol based and mono- and oligobutyleneglycol based chains, i.e., chains that are based on the repeating unit —CH₂CH₂O—, —CH₂CH₂CH₂O— or —CH₂CH₂CH₂CH₂O—. The alkyleneglycol moiety may be monodisperse (with a well-defined length/molecular weight). Monoalkylene glycol moieties comprise —OCH₂CH₂O—, —OCH₂CH₂CH₂O— or —OCH₂CH₂CH₂CH₂O— containing different groups at each end.

The connections between the moieties Acy, AA 1, AA2 and/or AA3 are formally obtained by amide bond (peptide bond) formation (—CONH—) by removal of water from the parent compounds from which they formally are built.

For example, a suitable albumin binder comprising an acyl moiety is Eicosandioyl-gGlu-(OEG)₂. In Eicosandioyl-gGlu-(OEG)₂, the functional group capable of binding to albumin is the terminal COOH group of the eicosandiol group and the albumin binder may be coupled to the insulin polypeptide via a terminal OEG group:

HOOC—(CH₂)₁₈—C(═O)—NH—CH(COOH)—(CH₂)₂—C(═O)—NH—(CH₂)₂—O—(CH₂)₂—O—CH₂—C(═O)—NH—(CH₂)₂—O—(CH₂)₂—O—C(═O)—NH— insulin polypeptide.

Suitable acyl moieties are described in WO 2009/115469 A1 (Novo Nordisk, published 24 Sep. 2009) from page 27, line 13 to page 43.

Optionally, the albumin binder is a sulfonamide of Formula (I) as described in detail in section A above.

According to step a) of the process of the present invention described in Section B, a proinsulin shall be provided. In some embodiments, the provided proinsulin has been produced by expressing a polynucleotide encoding for said proinsulin in a host cell. The thus produced proinsulin may have been subsequently purified from the cultivation medium in which the host cell was cultivated.

The proinsulin in accordance with the present invention shall comprise from N- to C-terminus an insulin B-chain, a linker peptide and an insulin A-chain. Accordingly, the proinsulin comprises a B-chain fused to linker peptide followed by the C-terminal A-chain. The insulin B-chain, the linker peptide and the insulin A-chain shall be linked via peptide bonds, typically without intervening amino acid residues. Suitable proinsulins are described herein below.

In accordance with the present invention, the linker peptide shall have a length of at least one amino acid residues, such as of least two amino acid residues. For example, the linker peptide may have a length in the range 1 to 30 amino acid residues, in particular, in the range from 2 to 30 amino acid residues. In some embodiments, the linker has a length in the range from 4 to 9 amino acid residues. In some embodiments, the linker peptide has a length of four amino acid residues.

In at least one embodiment, the first amino acid of the linker peptide is selected from the group consisting of an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and a valine residue. In at least one embodiment, the first amino acid of the linker peptide is selected from the group consisting of an alanine, arginine, asparagine, aspartic acid, glutamine, glutamic acid, glycine, histidine, isoleucine, leucine, methionine, phenylalanine, proline, serine, threonine, tryptophan, tyrosine, and a valine residue.

In some embodiments, the first amino acid of the linker peptide is threonine, phenylalanine residue, a glutamine residue, a glutamic acid residue, an asparagine residue or an aspartic acid residue. The aforementioned amino acid residues have a low nucleophilicity. For example, the nucleophilicity of the aforementioned amino acid residues is lower than the nucleophilicity of glycine which can be found in many insulin analogs at the A1 position. Accordingly, the first amino acid of the insulin A chain is a glycine residue.

In some embodiments, the first amino acid of the linker peptide is threonine. Further, it is envisaged that the last amino acid of the linker peptide is arginine. The presence of an arginine residue at this position allows for the removal of the linker peptide with trypsin in step d) of the above method.

Accordingly, the linker peptide, i.e. the linker peptide between the B chain and the A-chain, may comprise or, in particular, consist of the following sequence:

(SEQ ID NO: 106)
Xaa1-Xaa2-Xaa3-Xaa4-Xaa5-Xaa6-Xaa7-Xaa8-Arg

wherein
Xaa1 is any naturally occurring amino acid residue, for example wherein Xaa1 is threonine, phenylalanine, glutamine, glutamic acid, asparagine or aspartic acid,
Xaa2 is any naturally occurring amino acid residue or Xaa2 is absent. In some embodiments, Xaa2 is glutamic acid.
Xaa3 is any naturally occurring amino acid residue, in particular wherein Xaa3 is glycine, or wherein Xaa3 is absent,
Xaa4 is any naturally occurring amino acid residue, or Xaa4 is absent,
Xaa5 is any naturally occurring amino acid residue, or Xaa5 is absent,
Xaa6 is any naturally occurring amino acid residue, or Xaa6 is absent,
Xaa7 is any naturally occurring amino acid residue, or n Xaa7 is absent, and
Xaa8 is any naturally occurring amino acid residue, or Xaa8 is absent.

Xaa1, Xaa2, Xaa3, Xaa4, Xaa5, Xaa6, Xaa7 and Xaa8 can be any naturally occurring amino acid residue, in particular alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, Xaa1, Xaa2, Xaa3, Xaa4, Xaa5, Xaa6, Xaa7 and Xaa8 are not lysine and cysteine. In this case, Xaa1, Xaa2, Xaa3, Xaa4, Xaa5, Xaa6, Xaa7 and Xaa8 are (independently) selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine.

Thus, Xaa1 to Xaa8 may be as follows:
Xaa1 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. In some embodiments, Xaa1 is threonine, phenylalanine, glutamine, glutamic acid, asparagine or aspartic acid.
Xaa2 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, or valine. In some embodiments, Xaa2 is glutamic acid. Alternatively, Xaa2 is absent.
Xaa3 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. In some embodiments, Xaa3 is glycine. Alternatively, Xaa3 is absent.
Xaa4 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa4 is absent,
Xaa5 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa5 is absent,
Xaa6 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa6 is absent,
Xaa7 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa7 is absent,
Xaa8 may be selected from the group consisting of alanine, arginine, asparagine, aspartic acid, glutamine, glutamic acid, glycine, histidine, isoleucine, leucine, methionine, phenylalanine, proline, serine, threonine, tryptophan, tyrosine, and valine. Alternatively, Xaa8 is absent.

In some embodiments, the linker peptide consists of the sequence Xaa1-Arg, wherein Xaa1 has a meaning as set forth above, e.g. wherein Xaa1 is threonine, phenylalanine, glutamine, glutamic acid, asparagine or aspartic acid. In some embodiments, the linker peptide consists of the sequence Thr-Arg.

In an alternative embodiment, the linker peptide consists of the sequence Xaa1-Xaa2-Arg, such as of Thr-Xaa2-Arg, e.g. Thr-Glu-Arg.

In an alternative embodiment of the present invention, the linker peptide consists of a sequence Xaa1-Xaa2-Xaa-3-Arg (SEQ ID NO: 114), such as of Thr-Xaa2-Xaa3-Arg (SEQ ID NO: 115), e.g. of Thr-Glu-Gly-Arg (SEQ ID NO: 112). Thus, the linker peptide may be TEGR (SEQ ID NO: 112).

As set forth above, the proinsulin provided in step a) of the process described in this section shall also comprise an insulin A-chain and insulin B-chain. For example, the insulin A-chain and insulin B-chain may be an insulin A-chain and insulin B-chain of an insulin analog described in Section A of the present application. In some embodiments, the insulin A-chain is the A-chain of any one of the insulin analogs listed in Table 1, and the insulin B-chain is the corresponding insulin B-chain. For example, the proinsulin may comprise the A-chain and the B-chain of insulin analog 24 shown in Table 1.

In some embodiments, the proinsulin comprises an A chain which comprises or, in particular, consists of the following sequence:

(SEQ ID NO: 109)
GIVEQCCTSICSL Xaa9 QLENYCN,

wherein Xaa9 is glutamic acid (Glu), aspartic acid (Asp) or histidine (His)

Further, the proinsulin may comprise a B chain which comprises or, in particular, consists of following sequence:

(SEQ ID NO: 110)
FVNQHLCGSHLVEAL Xaa10 LVCGERGF Xaa11 YTPK,

wherein Xaa10 is tyrosine (Tyr), valine (Val), isoleucine (Ile), leucine (Leu), alanine (Ala) or histidine (His), and/or
wherein Xaa11 is phenylalanine (Phe), valine (Val), isoleucine (Ile), leucine (Leu), alanine (Ala) or histidine (His).

In some embodiments, Xaa9 is glutamic acid (Glu), Xaa10 is tyrosine (Tyr), and Xaa11 is valine (Val), isoleucine (Ile), or leucine (Leu). In some embodiments, Xaa9 is glutamic acid (Glu), Xaa10 is tyrosine (Tyr), and Xaa11 is valine (Val).

In some embodiments, the proinsulin provided in step a) of the above process has the following sequence:

(SEQ ID NO: 111)
FVNQHLCGSHLVEAL Xaa10 LVCGERGF Xaa11 YTPK Xaa1
Xaa2 Xaa3 Xaa4 Xaa5 Xaa6 Xaa7 Xaa8 R
GIVEQCCTSICSL Xaa9 QLENYCN,

wherein Xaa1 to Xaa11 have the meanings as set forth above.

In the above sequence, the B-chain is indicated in bold, the linker peptide is indicated in italics and the A chain is underlined.

In some embodiments, the proinsulin comprises an A-chain consisting of the sequence GIVEQCCTSICSLEQLENYCN (SEQ ID NO: 47) and a B chain consisting of the sequence shown in FVNQHLCGSHLVEALYLVCGERGFVYTPK (SEQ ID NO: 48). Further, the linker peptide between the B-chain and the A chain may have a sequence as set forth above, e.g. as shown in SEQ ID NO: 106. In some embodiments, the linker peptide has the sequence TEGR (SEQ ID NO: 112). For example, the proinsulin provided in step a) of the above process may have the following sequence:

(SEQ ID NO: 108)
FVNQHLCGSHLVEALYLVCGERGFVYTPKTEGRGIVEQCCTSICSLEQL
ENYCN

Again, the B-chain is indicated in bold, the linker peptide is indicated in italics and the A chain is underlined.

According to step b) of the process described in Section B, the proinsulin provided in step a) is cleaved with a first protease between the last amino acid of the insulin B-chain and the first amino acid of the linker peptide. By cleaving the proinsulin with the protease, an insulin precursor is generated comprising the insulin B-chain and an N-terminally extended A-chain.

Said N-terminally extended A-chain comprises, from N to C-terminus, the linker peptide and the A-chain (fused via a peptide bond). Accordingly, the N-terminally extended A-chain comprises at the N-terminus, depending on the length of the linker peptide, one or more additional amino acid residues as compared to the A-chain of the mature insulin, for example 2 to 30 additional amino acid residues, such as 4 to 9 additional amino acid residues.

In some embodiments, the N-terminally extended A-chain comprises at the N-terminus the entire linker peptide. Accordingly, the first amino acid of the linker peptide shall be the first amino acid of the N-terminally extended A-chain.

As set forth above, the insulin precursor generated by cleavage with the first protease shall comprise i) the insulin B-chain and ii) an N-terminally extended A-chain comprising the linker peptide and the A-chain. In the N-terminally extended A-chain, the linker peptide is still fused to the A-chain via a peptide bond, whereas the B-chain is no longer bound to the linker peptide via a peptide bond. However, the B-chain and the N-terminally extended A-chain may be connected by disulfide bridges between cysteine residues, for example, by one disulfide bridge between the cysteine at position A7 of the A-chain and the cysteine at position B7 of the B-chain, and by one disulfide bridge between the cysteine at position A20 of the A-chain and the cysteine at position B19 of the B-chain.

In accordance with the process described in this section, the first protease shall be a capable of cleaving the peptide bond between the B-chain and the linker peptide in the provided proinsulin, i.e. the peptide bond between the last amino acid residue of the B-chain and the first amino acid residue of the linker peptide in the A-chain. Therefore, the first protease is to be chosen that it allows for the cleavage of said peptide bond.

The term “protease” as used herein is synonymous with peptidase or proteinase. The term refers to a protein that catalyzes the cleavage of peptide bonds in peptides/polypeptides. Examples of proteases include trypsin, TEV protease (Tobacco Etch Virus protease) and endoproteinase Lys-C.

In an embodiment of the process of the present invention, the first protease is endoproteinase Lys-C. Endoproteinase Lys-C is a serine endoproteinase which cleaves peptide bonds at the carboxyl side of lysine. Thus, in order to allow for the cleavage of the proinsulin by endoproteinase Lys-C in step b) of the process described in Section B, the last amino acid, i.e. the C-terminal amino acid, of the B chain shall be lysine. For example, it is envisaged that the B chain comprises lysine at position B29, but lacks the amino acid at position B30. Thus, the B-chain comprised by the proinsulin as referred to in step a) of the above described proinsulin may be a des(B30) B-chain.

It is to be understood that the cleavage with the first protease and the cleavage with the second protease in steps b) and d) of the process described in Section B are carried out under conditions which allow for the cleavage. Such conditions are well-known in the art and can be selected by the skilled person without further ado.

The insulin precursor as referred to herein in Section B shall comprise a free amino group. In some embodiments, the free amino group is the amino group of a lysine comprised in the precursor, such as a terminal lysine, for example a lysine present at a C terminus of the B chain, such as position B29 of the B-chain.

In some embodiments, the terminal lysine is the only lysine residue present in insulin precursor generated by cleavage of the proinsulin with the first protease.

After cleavage with the first protease, the produced insulin precursor shall be contacted with an albumin binder in step c) of the process described in Section B). The albumin binder is as defined above in connection with the process of the present invention.

In step c) of the process of the present invention described in Section B, a conjugate of an albumin binder and the insulin precursor is generated. In some embodiments, a conjugate as described in the method of the present invention in Section A is generated. Thus, the, optionally activated, albumin binder to be used in step c) of the process of the present invention described in Section B may be an activated albumin binder as described in Section A above.

According to step c) a conjugate of an albumin binder and the insulin precursor is generated. In at least one embodiment, the insulin precursor is covalently bond to the albumin binder. For example, where the albumin binder is an activated albumin binder, which comprises an activated carboxyl group —COOR^{x} in the pre-coupled state, the terminal carboxy group carrying the R^{x} group in the non-conjugated state is covalently bond to a suitable functional group of the insulin precursor, for example to an amino group or a hydroxyl group of the insulin precursor. It goes without saying that in case of an amide bond formed with an amino group of the insulin precursor, the carboxyl group carrying the R^{x} group in the pre-coupled state is present in the conjugate formed as carbonyl group —C(═O)—, i.e. an amide bond —C(═O)—NH— is formed wherein —C(═O) is the remaining part of the COOR^{x} group and —NH— is the remaining part of the insulin precursor's amino group. For example, the amino group is from a lysine present at a C terminus of the B chain, such as position B29 of the B-chain. After generating a conjugate of an albumin binder and the insulin precursor by step c) of the process of the present invention described in section B), the generated conjugate is contacted in step d) with a second protease. Step d) shall allow for the proteolytic cleavage of the N-terminally extended A-chain of said insulin precursor comprised by the conjugate generated in step d) between the last amino acid of the linker peptide and the first amino acid of the A-chain, thereby generating a conjugate of an albumin binder and an mature insulin, in particular a conjugate of an albumin binder and an insulin analog as set forth herein, such as an insulin analog disclosed in Section A.

Accordingly, the N-terminally extended A-chain shall be cleavable by the second protease between the linker peptide and the A-chain and, thus, shall comprise a cleavage site for the second protease between the last amino acid of the linker peptide and the first amino acid of the A-chain. Cleavage of the N-terminally extended A-chain with said second protease shall result in the A-chain and the linker peptide, wherein the A-chain and the linker peptide are no longer covalently bound via a peptide bond.

In some embodiments, the second protease to be used in step b) of the process described in Section B herein is trypsin or a TEV protease (Tobacco Etch Virus protease). Accordingly, the first protease may be endoproteinase Lys-C and the second protease may be trypsin or a TEV protease (Tobacco Etch Virus protease).

In some embodiments, the second protease is trypsin. Trypsin (EC 3.4.21.4) is a serine protease from the PA clan superfamily, found in the digestive system of many vertebrates, where it hydrolyzes proteins. In some embodiments, trypsin is a vertebrate trypsin, such as a mammalian trypsin, e.g. porcine trypsin. Other names for trypsin are a-trypsin; 8-trypsin, pseudotrypsin, tryptase, tripcellim, sperm receptor hydrolase. Trypsin cleaves the peptide bond after an arginine residue or after a lysine residue (Arg-|-Xaa, Lys-|-Xaa). The trypsin to be used herein can be of any sources, such trypsin from bovine pancreas, trypsin from human pancreas, trypsin from porcine pancreas, recombinantly produced trypsin, or mutated trypsin (such as a trypsin described in WO2006015879A1 [Roche, Hoess] or WO2007031187A1 [Sanofi-Aventis, Geipel]) as long as it is capable of cleaving a peptide or polypeptide, such as the N-terminally extended A-chain as set forth herein, after an arginine residue and/or after a lysine residue. Accordingly, the last amino acid of the linker peptide is typically an arginine residue or a lysine residue. In some embodiments, the last amino acid of the linker peptide is an arginine residue (e.g. when the first protease is LysC).

In accordance with the present invention, it is envisaged that the proinsulin provided in step a) of the process described in Section B may further comprise a signal peptide, for example a signal peptide that allows for secretion of a proinsulin produced by a host cell into the cultivation medium. Suitable signal peptides are known in the art and are selected depending on the chosen expression host. In particular, said proinsulin may comprise a signal peptide at the N-terminus of the proinsulin, i.e. N-terminally to the insulin B chain. Thus, the order shall be as follows (from N- to C-terminus): signal peptide, B-chain, linker peptide, A chain (all linked via peptide bonds). The signal peptide comprised by the proinsulin is removed in step b) by cleavage with the first protease. Accordingly, the first protease additionally cleaves the proinsulin between the last amino acid of the signal peptide and the first amino acid of B-chain. Thus, the proinsulin comprises two cleavage sites for the first protease, one cleavage site between the signal peptide and the B-chain, and one cleavage site between the B-chain of the linker peptide. Cleavage with the first protease in step b) of the above process of the present invention will generate a signal peptide and an insulin precursor comprising a B-chain and an N-terminally extended A chain (as described elsewhere herein). Thus, the signal peptide and the B-chain are no longer covalently bound by a peptide bond. The same applies to the B-chain and the N-terminally extended A-chain.

As set forth above, the first protease may be endoproteinase Lys-C. Thus, the last amino acid of the signal peptide may be a lysine residue. In some embodiments, the last amino acid of the signal peptide and the last amino acid of the B is a lysine residue. The presence of a lysine residue at these positions allows for the cleavage with endoproteinase Lys-C.

The generated signal peptide is no longer needed and can be removed. The generated insulin precursor can be subsequently further processed in step c). Accordingly, the precursor is contacted with an activated albumin binder as described elsewhere herein.

In step d) of the process of the present invention, a conjugate of an albumin binder and a mature insulin is produced. Accordingly, a conjugate of an albumin binder and an insulin analog (such as an insulin analog as disclosed in Section A or Table 4) is produced. In some embodiments, the conjugate is the conjugate shown in FIG. 3. In some embodiments, the conjugate is the conjugate shown in FIG. 4. In some embodiments, the conjugate is the conjugate shown in FIG. 5. In some embodiments, the conjugate is the conjugate shown in FIG. 6.

The definitions given herein above in connection with the process described in this section apply mutatis mutandis to the following.

The present invention also relates to a proinsulin comprising from N- to C-terminus:
(a) an insulin B-chain,
(b) a linker peptide, and
(c) an insulin A-chain,
wherein said proinsulin comprises a cleavage site for endoproteinase Lys-C between the last amino acid of the insulin B-chain and the first amino acid of the linker peptide and a cleavage site for trypsin between the last amino acid of the linker peptide and the first amino acid of the insulin A-chain. Accordingly, the last amino acid residue of the insulin B-chain is a lysine residue. Further, the last amino acid of linker peptide is an arginine residue.

In some embodiments, the first amino acid of the A-chain is a glycine residue.

In some embodiments, the proinsulin further comprises N-terminally to the insulin B-chain a signal peptide. Accordingly, the proinsulin comprises a further cleavage site for endoproteinase Lys-C between the last amino acid of the signal peptide and the first amino acid of B-chain. Accordingly, the last amino acid residue of the signal peptide is a lysine residue.

The present invention also relates to a polynucleotide coding for the proinsulin according to the present invention. In some embodiments, the polynucleotide is operably linked to a promoter. Said promoter shall allow for expressing said polynucleotide in a host cell. In some embodiments, the promoter is heterologous with respect to the polynucleotide.

The present invention further relates to a vector comprising the polynucleotide of the present invention. In some embodiments, said vector is an expression vector.

The present invention also relates to host cell comprising the proinsulin of the present invention, the polynucleotide of the present invention and/or the vector of the present invention. In some embodiments, the host cell is a bacterial cell such as a cell of belonging to the genus Escherichia, e.g. an E. coli cell. In another embodiment, the host cell is a yeast cell, such as a Pichia pastoris cell or Klyveromyces lactis cell.

The present invention also concerns an N-terminally extended insulin A-chain as set forth herein above in step a) of the process of the invention described in Section B. Specifically, the N-terminally extended insulin A-chain comprises from N- to C-terminus:
(a) a linker peptide, and
(b) an insulin A-chain,
wherein said proinsulin comprises a cleavage site for trypsin between the last amino acid of the linker peptide and the first amino acid of the A-chain.

Typically, the last amino acid of the linker peptide is an arginine residue. Further, it is envisaged that the first amino acid of the insulin A chain is a glycine residue.

In some embodiments, the insulin A-Chain comprises or consists of the sequence GIVEQCCTSICSL Xaa9 QLENYCN (SEQ ID NO: 109), wherein Xaa9 is glutamic acid (Glu), aspartic acid (Asp) or histidine (His), e.g. wherein Xaa9 is glutamic acid (Glu) and the linker peptide comprises of consists of the sequence Xaa1-Xaa2-Xaa3-Xaa4-Xaa5-Xaa6-Xaa7-Xaa8-arginine (SEQ ID NO: 106), wherein Xaa1 to Xaa8 have the meanings set forth herein above in this section.

In some embodiments, the N-terminally extended A chain comprises or, in particular, consists of the sequence

(SEQ ID NO: 113)
Xaa1 Xaa2 Xaa3 Xaa4 Xaa5 Xaa6 Xaa7 Xaa8 R
GIVEQCCTSICSL Xaa9 QLENYCN,

wherein Xaa1 to Xaa9 have the meanings as set forth above. In some embodiments, the N-terminal extended A-chain comprises or consists of the sequence

(SEQ ID NO: 107)
TEGRGIVEQCCTSICSLEQLENYCN.

The present invention further pertains to an insulin precursor comprising the N-terminally extended insulin A-chain of the present invention and an insulin B-chain.

The insulin B chain comprised by the precursor shall not be bound to the N-terminally extended insulin A-chain via a peptide bond. Further, it may be any B chain as set forth herein. In some embodiments, the insulin-B chain is a des(B30) B-chain. Accordingly, the amino acid at position 30 is absent. In some embodiments, the N-terminal amino acid is lysine at position B29.

In some embodiments, the B chain comprises of consists of the sequence:

(SEQ ID NO: 110)
FVNQHLCGSHLVEAL Xaa10 LVCGERGF Xaa11 YTPK,

wherein Xaa10 is tyrosine (Tyr), valine (Val), isoleucine (Ile), leucine (Leu), alanine (Ala) or histidine (His), and/or
wherein Xaa11 is phenylalanine (Phe), valine (Val), isoleucine (Ile), leucine (Leu), alanine (Ala) or histidine (His).

In some embodiments, the insulin precursor comprises a B chain comprising or consisting of the sequence FVNQHLCGSHLVEALYLVCGERGFVYTP (SEQ ID NO: 48).

Thus, the insulin precursor may have the sequence shown in FIG. 7 (B-chain: SEQ ID NO: 48, N-terminally extended A-chain: SEQ ID NO: 107):

The present invention further concerns a conjugate comprising the insulin precursor of and a sulfonamide as set forth herein in section B. In some embodiments, the conjugate is as shown in FIG. 8.

Finally, the present invention relates to a process for generating an insulin precursor, said process comprising
a) Providing a proinsulin comprising from N- to C-terminus an insulin B-chain, a linker peptide and an insulin A-chain,
b) Cleaving the proinsulin provided in step a) with a first protease between the last amino acid of the insulin B-chain and the first amino acid of the linker peptide, thereby generating an insulin precursor, said insulin precursor comprising the insulin B-chain and an N-terminally extended A-chain comprising the linker peptide and the A-chain.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e.

the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and exemplary aspects of the present invention.
1. A method of forming a conjugate of a sulfonamide and a polypeptide, the method comprising:
   a) Providing an activated sulfonamide of Formula (I):

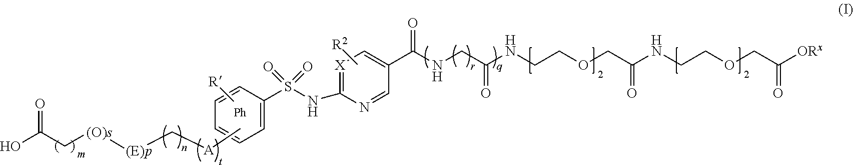

wherein
A is selected from the group consisting of oxygen atom, —CH₂CH₂— group, —OCH₂— group and —CH₂O— group;
E represents a —C₆H₃R— group with R being a hydrogen atom or a halogen atom, wherein the halogen atom is selected from the group consisting of fluorine, chlorine, bromine and iodine atom;
X represents a nitrogen atom or a —CH— group;
m is an integer in the range from 5 to 17;
n is zero or an integer in the range from 1 to 3;
p is zero or 1;
q is zero or 1;
r is an integer in the range from 1 to 6;
s is zero or 1;
t is zero or 1;
R¹ represents at least one residue selected from the group of hydrogen atom, halogen atom, C1 to C3 alkyl group and halogenated C1 to C3 alkyl group;
R² represents at least one residue selected from the group of hydrogen atom, halogen atom, C1 to C3 alkyl group and halogenated C1 to C3 alkyl group;
R^{x} represents an activation group;
wherein the combination of s being 1, p being zero, n being zero, A being an oxygen atom and t being 1 is excluded for Formula (I);
b) Providing an aqueous solution of a polypeptide having a free amino group, wherein the aqueous solution optionally comprises an alcohol;
c) Contacting the aqueous solution of b) with the activated sulfonamide of a); and
d) Reacting the activated sulfonamide with the polypeptide having a free amino group, obtaining a solution comprising the conjugate of a sulfonamide and a polypeptide, wherein the sulfonamide is covalently bonded to the polypeptide.
2. The method of embodiment 1, wherein the polypeptide having a free amino group is a polypeptide or a precursor thereof, each having a free amino group, wherein the precursor of the polypeptide comprises an additional sequence of one or more further amino acid residues compared to the polypeptide.
3. The method of embodiment 1 or 2, wherein the aqueous solution provided in a) has a pH value in the range of from 9 to 12 or in the range of from 9.5 to 11.5, or in the range of from 10 to 11, wherein the pH value is determined with a pH sensitive glass electrode according to ASTM E 70:2007.
4. The method of any one of embodiments 1 to 3, wherein the pH value is adjusted in the respective range by addition of a base, or a base selected from the group consisting of alkali hydroxides (lithium hydroxide, sodium hydroxide, potassium hydroxide), alkyl amines and mixtures of two or more thereof; or selected from the group of tertiary alkyl amines N(C1-C5 alkyl)₃, primary alkyl amines H₂N—C(C1-C5 alkyl)₃ and mixtures of two or more thereof, wherein the C1-C5 alkyl groups of the tertiary amines and of the primary amines are each independently selected from branched or straight C1-C5 alkyl groups and wherein each C1-C5 alkyl group has at least one substituent selected from the group of hydrogen atom, hydroxyl group and carboxyl group; or selected from the group of tertiary alkyl amines N(C1-C3 alkyl)₃, primary alkyl amines H₂N—C(C1-C3 alkyl)₃ and mixtures of two or more thereof, wherein the C1-C3 alkyl groups of the tertiary amines and of the primary amines are each independently selected from branched or straight C1-C3 alkyl groups and wherein each C1-C3 alkyl group has at least one substituent selected from the group of hydrogen atom, hydroxyl group and carboxyl group; or selected from the group of bicine, trimethylamine, trimethylamine, tris(hydroxymethyl)aminomethane and mixtures of two or more thereof; wherein the base in particular comprises at least triethylamine.
5. The method of any one of embodiments 1 to 4, wherein contacting the aqueous solution of b) with the activated sulfonamide of a) according to step c) is done in that the activated sulfonamide of a) is added as a solution of the activated sulfonamide to the aqueous solution of b).
6. The method of embodiment 5, wherein the solution of the activated sulfonamide is an organic solution.
7. The method of embodiment 6, wherein the organic solution comprises the activated sulfonamide and a polar aprotic organic solvent,
8. The method of embodiment 7, wherein the polar aprotic organic solvent has an octanol-water-partition coefficient (K_{OW}) in the range of from 1 to 5 at standard conditions (T: 20-25° C., p: 1013 mbar); or wherein the polar aprotic organic solvent is selected from the group consisting of tetrahydrofuran, acetonitrile, dimethylformamide, and mixtures of two or more thereof; in particular selected from the group of tetrahydrofuran, acetonitrile and mixtures of tetrahydrofuran and acetonitrile.
9. The method of any one of embodiments 1 to 8, wherein contacting the aqueous solution of b) with the activated sulfonamide of a) according to step c) is done in that the activated sulfonamide of a) is added in solid form to the aqueous solution of b) or at least partially in crystalline form, or at least 90 weight-% in crystalline form.
10. The method of any one of embodiments 2 to 9, wherein step d) comprises:
   d.1) Reacting the activated sulfonamide with a precursor of the polypeptide having a free amino group at a pH in the range from 9 to 12, obtaining a pre-conjugate comprising the sulfonamide and the precursor of the polypeptide, wherein the sulfonamide is covalently bonded to the precursor of the polypeptide by an amide bond C(═O)—NH— formed between the —C(═O)—O(R) of the (activated) sulfonamide of Formula (I) and the amino group of the precursor of the polypeptide;
   d.2) Enzymatic digestion at a pH in the range below 9 of the precursor of the polypeptide of the pre-conjugate obtained according to d.1), obtaining a solution comprising the conjugate of the sulfonamide and the polypeptide.
11. The method of any one of embodiments 2 to 10, further comprising:
   e) Isolating the conjugate of the sulfonamide and the polypeptide from the solution obtained in d) or d.2).
12. The method of any one of embodiments 1 to 11, wherein the activation group R^{x} of the activated sulfonamide of Formula (I) is selected from the group consisting of 7-azabenzotriazole (for example derived from HATU or H BTU), 4-nitro benzene and N-succinimidyl-group, or wherein R^{x} is a N-succinimidyl-group.
13. The method of any one of embodiments 1 to 12, wherein the aqueous solution of the polypeptide having a free amino group according to b) comprises an alcohol which is selected from the group consisting of C1-C4 monoalcohols and mixtures of two or more thereof, or from the group consisting of methanol, ethanol, propan-2-ol, propan-1-ol, butan-1-ol and mixtures of two or more thereof, or from the group consisting of ethanol, propan-2-ol, propan-1-ol, and mixtures of two or more thereof.
14. The method of any one of embodiments 1 to 13, wherein the aqueous solution according to b) comprises an alcohol, wherein the alcohol is present in the aqueous solution in an amount in the range from 0.0001 to 35 volume-%, or in the range from 0.001 to 30 volume-%, or in the range from 0.01 to 25 volume-%, or in the range from 0.1 to 20 volume-%, each based on the total volume of water and alcohol.
15. The method of any one of embodiments 6 to 14, wherein the enzymatic digestion according to d.2) comprises use of at least one enzyme selected from the group consisting of trypsin, a TEV protease (Tobacco Etch Virus protease) and mixtures of trypsin and TEV protease.
16. The method of any one of embodiments 1 to 15, wherein the polypeptide is a mature insulin, which comprises an A chain and a B chain, wherein the A chain comprises at least one mutation relative to the A chain of human insulin and/or the B chain comprises at least mutation relative to human insulin,
   for example wherein the at least one mutation relative to the A chain of human insulin is a substitution at position A14, such as a substitution with an amino acid selected from the group consisting of glutamic acid (Glu), aspartic acid (Asp) and histidine (His), and/or a substitution at position A21, such as a substitution with glycine (Gly), for example wherein the mutation relative to the B chain of human insulin is a substitution at position B16, such as a substitution with an amino acid selected from the group consisting of valine (Val), isoleucine (Ile), leucine (Leu), alanine (Ala) or histidine (His), a substitution at position B25, such as a substitution with valine (Val), isoleucine (Ile), leucine (Leu), alanine (Ala) or histidine (His), and/or a deletion at position B30,
   in particular wherein the mature insulin is selected from the group consisting of
   Leu(B16)-human insulin,
   Val(B16)-human insulin,
   Ile(B16)-human insulin,
   Leu(B16)Des(B30)-human insulin,
   Val(B16)Des(B30)-human insulin,
   Ile(B16)Des(B30)-human insulin,
   Leu(B25)-human insulin,
   Val(B25)-human insulin,
   Ile(B25)-human insulin,
   Leu(B25)Des(B30)-human insulin,
   Val(B25)Des(B30)-human insulin,
   Ile(B25)Des(B30)-human insulin,
   Glu(A14)Leu(B16)Des(B30)-human insulin,
   Glu(A14)Ile(B16)Des(B30)-human insulin,
   Glu(A14)Val(B16)Des(B30)-human insulin,
   Glu(A14)Leu(B16)-human insulin,
   Glu(A14)Ile(B16)-human insulin,
   Glu(A14)Val(B16)-human insulin,
   Glu(A14)Leu(B25)Des(B30)-human insulin,
   Glu(A14)Ile(B25)Des(B30)-human insulin,
   Glu(A14)Val(B25)Des(B30)-human insulin,
   Glu(A14)Leu(B25)-human insulin,
   Glu(A14)Ile(B25)-human insulin,
   Glu(A14)Val(B25)-human insulin,
   Glu(A14)Gly(A21)Glu(B3)Val(B25)Des(B30)-human insulin,
   Glu(A14)Ile(B16)Ile(B25)Des(B30)-human insulin,
   Glu(A14)Glu(B3)Ile(B16)Ile(B25)Des(B30)-human insulin,
   Glu(A14)Ile(B16)Val(B25)Des(B30)-human insulin,
   Glu(A14)Gly(A21)Glu(B3)Ile(B16)Val(B25)Des(B30)-human insulin,
   Glu(A14)Val(B16)Ile(B25)Des(B30)-human insulin,
   Glu(A14)Val(B16)Val(B25)Des(B30)-human insulin,
   Glu(A14)Glu(B3)Val(B16)Val(B25)Des(B30)-human insulin,
   Glu(A14)Gly(A21)Glu(B3)Val(B16)Val(B25)Des(B30)-human insulin,
   Glu(A14)Gly(A21)Glu(B3)Val(B25)-human insulin,
   Glu(A14)Ile(B16)Ile(B25)-human insulin,
   Glu(A14)Glu(B3)Ile(B16)Ile(B25)-human insulin,
   Glu(A14)Ile(B16)Val(B25)-human insulin,
   Glu(A14)Gly(A21)Glu(B3)Ile(B16)Val(B25)-human insulin,
   Glu(A14)Val(B16)Ile(B25)-human insulin,
   Glu(A14)Val(B16)Val(B25)-human insulin,
   Glu(A14)Glu(B3)Val(B16)Val(B25)-human insulin, and
   Glu(A14)Gly(A21)Glu(B3)Val(B16)Val(B25)-human insulin
   Glu (A14)His(B25)Des(B30) human insulin, and
   Glu (A14)His(B16)His(B25) Des(B30) human insulin.
17. The method of any one of embodiments 1 to 16, wherein the sulfonamide is covalently bonded to the polypeptide and the precursor thereof respectively by an amide bond C(═O)—NH— formed between the —C(═O)—O(R^{x}) of the (activated) sulfonamide of Formula (I) and the free amino group of the polypeptide and the precursor thereof respectively, wherein the free amino group of the polypeptide is optionally the amino group of a lysine comprised in a mature insulin and the precursor thereof respectively, such as a terminal lysine, in particular a lysine present at a C terminus of a mature insulin and the precursor thereof respectively, such as a lysine present at the C terminus of the B-chain.
18. The method of any one of embodiments 1 to 17, wherein the activated sulfonamide of Formula (I) is obtained or obtainable from a protected activated sulfonamide of Formula (0)

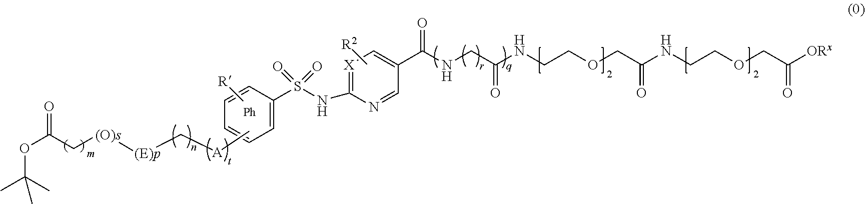

wherein A, E, X, m, n, p, q, r, s, t, R¹, R² and R^{x} have the meaning as defined in embodiment 1, wherein the protected activated sulfonamide of Formula (0) is deprotected by addition of one or more acids, or by addition of at least trifluoroacetic acid.
19. The method of any one of embodiments 1 to 18, wherein the precursor of the polypeptide comprises a sequence according to embodiment 12 and an additional linker peptide, which has a length of at least two amino acid residues, or a length in the range from 2 to 30 amino acid residues, or a length in the range from 4 to 9 amino acid residues.
20. The method of embodiment 19, wherein the first amino acid of the linker peptide is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or a valine residue, wherein the first amino acid of the linker peptide is for example a threonine, phenylalanine residue, a glutamine residue, a glutamic acid residue, an asparagine residue or an aspartic acid residue.
21. The method of embodiment 19 or 20, wherein the last amino acid of the linker peptide is an arginine residue.
22. The method of any one of embodiments 19 to 21, wherein the linker peptide comprises the following sequence

(SEQ ID NO: 106)
Xaa1-Xaa2-Xaa3-Xaa4-Xaa5-Xaa6-Xaa7-Xaa8-Arg,

wherein
Xaa1 is any naturally occurring amino acid residue, optionally wherein Xaa1 is threonine, phenylalanine, glutamine, glutamic acid, asparagine or aspartic acid,
Xaa2 is any naturally occurring amino acid residue, optionally wherein Xaa2 is glutamic acid, or wherein Xaa2 is absent,
Xaa3 is any naturally occurring amino acid residue, in particular wherein Xaa3 is glycine, or wherein Xaa3 is absent,
Xaa4 is any naturally occurring amino acid residue, or wherein Xaa4 is absent,
Xaa5 is any naturally occurring amino acid residue, or wherein Xaa5 is absent,
Xaa6 is any naturally occurring amino acid residue, or wherein Xaa6 is absent,
Xaa7 is any naturally occurring amino acid residue, or wherein Xaa7 is absent, and
Xaa8 is any naturally occurring amino acid residue, or wherein Xaa8 is absent.
23. The method of any one of embodiments 19 to 22, wherein the linker peptide has the sequence TEGR (SEQ ID NO: 112).
24. A conjugate obtained or obtainable from the method of any one of embodiments 1 to 23.
25. A precursor of a polypeptide comprising a sequence of a mature insulin according to embodiment 16 and an additional linker peptide as defined in any one of embodiments 19 to 23 covalently bonded to the N terminus of the mature insulin A-chain.
26. A procedure for crystallizing an activated sulfonamide corresponding to Formula (I)

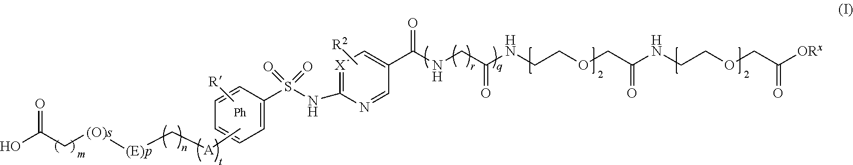

wherein A, E, X, m, n, p, q, r, s, t, R¹, R² and R^{x} have the meaning as defined in embodiment 1, comprising
   A) Providing a solution comprising the activated sulfonamide and an organic solvent;
   B) Removing the organic solvent at least partially, for example by distillation, obtaining a phase of the activated sulfonamide having a reduced amount of the organic solvent compared to the solution provided in A);
   C) Adding organic solvent to the phase obtained in B) obtaining a solution of the activated sulfonamide; and
   D) Repeating step B) with the solution obtained in C) obtaining a phase of the activated sulfonamide having a reduced amount of the organic solvent compared to the solution obtained in C);
   E) Optionally repeating steps C) and D) at least one further time.
27. The procedure of embodiment 26, wherein the solution comprising the activated sulfonamide and an organic solvent the organic solvent provided in A) further comprises trifluoroacetic acid.
28. The procedure of embodiment 26 or 27, wherein the organic solvent is selected from the group of organic solvents capable of forming an aceotropic mixture with trifluoroacetic acid.
29. The procedure of any one of embodiments 26 to 28, wherein the organic solvent is a polar aprotic organic solvent, which for example has an octanol-water-partition coefficient (K_{OW}) in the range from 1 to 5 at standard conditions (temperature: 20-25° C., pressure: 1013 mbar), wherein the organic solvent is for example selected from the group of acetonitrile, tetrahydrofuran and mixtures of acetonitrile and tetrahydrofuran, wherein the organic solvent in particular comprises at least comprises acetonitrile.
30. A solid form of the activated sulfonamide corresponding to Formula (I)

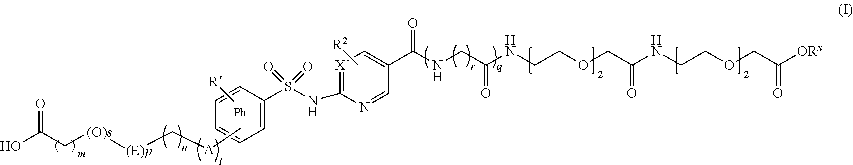

wherein A, E, X, m, n, p, q, r, s, t, R¹, R² and R^{x} have the meaning as defined in embodiment 1.
31. A process for generating a conjugate of an albumin binder and a mature insulin, said process comprising
   a) Providing a proinsulin comprising from N- to C-terminus an insulin B-chain, a linker peptide and an insulin A-chain,
   b) Cleaving the proinsulin provided in step a) with a first protease between the last amino acid of the insulin B-chain and the first amino acid of the linker peptide, thereby generating an insulin precursor, said insulin precursor comprising the insulin B-chain and an N-terminally extended A-chain comprising the linker peptide and the A-chain,
   c) Contacting said insulin precursor with a albumin binder, wherein the albumin binder comprises
      a functional group capable of binding to albumin;
      thereby generating a conjugate of an albumin binder and the insulin precursor,
   d) Cleaving the N-terminally extended A-chain of said insulin precursor comprised by the conjugate with a second protease between the last amino acid of the linker peptide and the first amino acid of the A-chain, thereby generating a conjugate of an albumin binder and a mature insulin.
32. The process of embodiment 31, wherein the last amino acid of the insulin B-chain is a lysine residue.
33. The process of embodiment 31 or 32, wherein the first amino acid of the A-chain is a glycine residue.
34. The process of any one of embodiments 31 to 33, wherein the linker peptide has a length of at least two amino acid residues.
35. The process of embodiment 34, wherein the linker peptide has length of 2 to 30 amino acid residues, such as a length of 4 to 9 amino acid residues.
36. The process of any one of embodiments 31 to 35, wherein the first amino acid of the linker peptide is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, e.g. wherein the first amino acid of the linker peptide is selected from alanine, arginine, asparagine, aspartic acid, glutamine, glutamic acid, glycine, histidine, isoleucine, leucine, methionine, phenylalanine, proline, serine, threonine, tryptophan, tyrosine.
37. The process of embodiment 36, wherein the first amino acid of the linker peptide is a threonine residue, phenylalanine residue, a glutamine residue, a glutamic acid residue, an asparagine residue or an aspartic acid residue, for example, wherein the first amino acid of the linker peptide is a threonine residue,
38. The process of any one of embodiments 31 to 37, wherein the last amino acid of the linker peptide is an arginine residue.
39. The process of any one of embodiments 31 to 38, wherein the first protease is endoproteinase Lys-C and/or wherein the second protease is trypsin or a TEV protease (Tobacco Etch Virus protease).
40. The process of any one of embodiments 31 to 39, wherein the proinsulin provided in step a) further comprises N-terminally to the insulin B-chain a signal peptide, and wherein the first protease additionally cleaves the proinsulin between the last amino acid of the signal peptide and the first amino acid of B-chain.
41. The process of embodiment 40, wherein the last amino acid of the signal peptide is a lysine residue.
42. A proinsulin comprising from N- to C-terminus:
   (a) an insulin B-chain,
   (b) a linker peptide, and
   (c) an insulin A-chain,
   wherein said proinsulin comprises a cleavage site for endoproteinase Lys-C between the last amino acid of the insulin B-chain and the first amino acid of the linker peptide and a cleavage site for trypsin between the last amino acid of the linker peptide and the first amino acid of the insulin A-chain.
43. The proinsulin of embodiment 42, wherein the proinsulin further comprises N-terminally to the insulin B-chain a signal peptide and wherein said proinsulin comprises a cleavage site for endoproteinase Lys-C between the last amino acid of the signal peptide and the first amino acid of B-chain.
44. The proinsulin of embodiment 43, wherein the linker peptide comprises the following sequence

(SEQ ID NO: 106)
Xaa1-Xaa2-Xaa3-Xaa4-Xaa5-Xaa6-Xaa7-Xaa8-Arg

wherein
Xaa1 is any naturally occurring amino acid residue, optionally wherein Xaa1 is threonine, phenylalanine, glutamine, glutamic acid, asparagine or aspartic acid,
Xaa2 is any naturally occurring amino acid residue, optionally wherein Xaa2 is glutamic acid, or wherein Xaa2 is absent,
Xaa3 is any naturally occurring amino acid residue, in particular wherein Xaa3 is glycine, or wherein Xaa3 is absent,
Xaa4 is any naturally occurring amino acid residue, or wherein Xaa4 is absent,
Xaa5 is any naturally occurring amino acid residue, or wherein Xaa5 is absent,
Xaa6 is any naturally occurring amino acid residue, or wherein Xaa6 is absent,
Xaa7 is any naturally occurring amino acid residue, or wherein Xaa7 is absent, and
Xaa8 is any naturally occurring amino acid residue, or wherein Xaa8 is absent.
45. The proinsulin according to any of embodiments 42 to 44, wherein the A-chain consists of the following sequence:

(SEQ ID NO: 109)
GIVEQCCTSICSL Xaa9 QLENYCN,

wherein Xaa9 is glutamic acid (Glu), aspartic acid (Asp) or histidine (His), and/or
wherein the B-chain consists of following sequence:

(SEQ ID NO: 110)
FVNQHLCGSHLVEAL Xaa10 LVCGERGF Xaa11 YTPK,

wherein Xaa10 is tyrosine (Tyr), valine (Val), isoleucine (Ile), leucine (Leu), alanine (Ala) or histidine (His), and/or
wherein Xaa11 is phenylalanine (Phe), valine (Val), isoleucine (Ile), leucine (Leu), alanine (Ala) or histidine (His).
46. The proinsulin of any one of embodiments 42 to 45, wherein the linker peptide has the sequence TEGR (SEQ ID NO: 112).
47. The proinsulin of embodiment 46, wherein the proinsulin comprises the following sequence

(SEQ ID NO: 111)
FVNQHLCGSHLVEAL Xaa10 LVCGERGF Xaa11 YTPK Xaa1
Xaa2 Xaa3 Xaa4 Xaa5 Xaa6 Xaa7 Xaa8 R
GIVEQCCTSICSL Xaa9 QLENYCN,

wherein Xaa1 to Xaa8 have the meaning as set forth in embodiment 40 or in Section B) above, and wherein Xaa9 to Xaa11 have the meaning as set forth in embodiment 41, for example wherein the proinsulin comprises the following sequence

(SEQ. ID NO: 108)
FVNQHLCGSHLVEALYLVCGERGFVYTPKTEGRGIVEQCCTSICSLEQL
ENYCN

48. A polynucleotide coding for the proinsulin according to any one of embodiments 42 to 47.
49. A vector comprising the polynucleotide of embodiments 48.
50. A host cell comprising the proinsulin according to any one of embodiments 42 to 47, the polynucleotide of embodiment 44 and/or the vector of embodiment 45.
51. An N-terminally extended insulin A-chain comprising from N- to C-terminus:
   (a) a linker peptide, and
   (b) an insulin A-chain,
   wherein said N-terminally extended insulin A-chain comprises a cleavage site for trypsin between the last amino acid of the linker peptide and the first amino acid of the A-chain.
52. An insulin precursor comprising the N-terminally extended insulin A-chain of embodiment 51 and an insulin B-chain.
53. A conjugate comprising the insulin precursor of embodiment 52 and a sulfonamide.
54. The conjugate of embodiment 53, wherein the conjugate is as (B-chain: SEQ ID NO: 48, N-terminally extended A-chain: SEQ ID NO: 107) is a conjugate as shown in FIG. 8.
55. The activated sulfonamide according to embodiment 30, wherein the activated sulfonamide is in crystalline form.
56. The method of any one of embodiments 1 to 23, being a method of forming a conjugate of a sulfonamide and an insulin polypeptide, optionally wherein the activated sulfonamide is an activated albumin binder.

The present invention is further illustrated by the following examples.

### Examples

I. Synthesis of Compounds & Preparation of Conjugates

All pH measurements were done with a pH sensitive glass electrode according to ASTM E 70:2007.

Yields from HPLC data were calculated from the integral relation between educt and product.

### 1 16 [4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate

### 1.1 Synthesis

32.5 g 2-[2-[2-[[2-[2-[2-[[2-[[4-(16-tert-butoxy-16-oxo-hexadecoxy)phenyl]sulfonylamino]-pyrimidine-5-carbonyl]amino]ethoxy]ethoxy]acetyl]amino]ethoxy]ethoxy]acetic acid (sticky syrup) was dissolved in 305 ml dry acetonitrile. 305 ml dry ethyl acetate under an argon layer, 16.25 g (63.4 mmol) N,N-disuccinimidylcarbonat and 3.25 g (26.6 mmol) dimethylaminopyridine was added under stirring at room temperature (25° C.). After 90 minutes, the solvent was distilled off using a rotary evaporator and the remaining oily product was dissolved in 1.5 liters of ethyl acetate. The ethyl acetate solution was extracted three times, each time with 300 ml 0.1 N HCl and 300 ml saturated NaCl solution. The solvent was distilled off using a rotary evaporator, leaving again an oily product. The oily product was dissolved in 0.5 liters of ethyl acetate, the precipitated NaCl was filtered off and the ethyl acetate was distilled off. Next, 0.5 liters of ethyl acetate was added and the solvent was distilled off using a rotary evaporator. This procedure—addition of 0.5 liters of ethyl acetate and removal of solvent by distillation—was repeated 3 times, resulting in an oily product. The oily product was dissolved in 325 ml methylene chloride. 163 ml trifluoroacetic acid was added and stirred at room temperature (25° C.) for 80 minutes. The solvent and the trifluoroacetic acid was distilled off using a rotary evaporator. To the remaining oily product 100 ml acetonitrile was added and the solvent was distilled off using a rotary evaporator. This procedure—addition of 100 ml acetonitrile and removal of the solvent by distillation—was repeated 6 times. Then 1.5 liters of acetonitrile was added and the solution was overlayed with argon and kept overnight at a temperature in the range of from 2 to 8° C. The resulting product in the acetonitrile solution under argon was only stable for less than 3 days). Yield in the acetonitrile solution was calculated from HPLC data as 78% based on the amount of 2-[2-[2-[[2-[2-[2-[[2[[4-(16-tert-butoxy-16-oxo-hexadecoxy)phenyl]sulfonylamino]-pyrimidine-5-carbonyl]amino]ethoxy]ethoxy]acetyl]amino]ethoxy]ethoxy]acetic acid used.

### 1.2 Crystallization

174.4 g (194.6 mmol) 2-[2[[2-[2-[[2-[[2-[[4-(16-tert-butoxy-16-oxo-hexadecoxy) phenyl]sulfonylamino]pyrimidine-5-carbonyl]amino]ethoxy]ethoxy]acetyl]amino]-ethoxy]ethoxy]acetic acid (sticky syrup) was dissolved in 2.3 liters of ethyl acetate at 45° C. 3.47 g (28.4 mmol) dimethylaminopyridine was added into an reaction vessel and subsequently the solution of 2-[2-[2-[[2-[2-[2-[[2[[4-(16-tert-butoxy-16-oxo-hexadecoxy)-phenyl]sulfonylamino]pyrimidine-5-carbonyl]amino]ethoxy]ethoxy]acetyl]amino]-ethoxy]ethoxy]acetic acid in ethyl acetate was added at room temperature (25° C.). Then a solution of 98.7 g (385.3 mmol) N,N-disuccinimidylcarbonate in 3.7 liters of acetonitrile was added under stirring at room temperature (25° C.). After 90 minutes the solvent was distilled off using a rotary evaporator and the remaining oily product was dissolved in 2.3 liters of ethyl acetate. The ethyl acetate solution was extracted three times, each with 470 ml 0.1 N HCl and 470 ml saturated NaCl solution. The solvent was distilled off using a rotary evaporator, leaving an oily product. The oily product was dissolved in 1.4 liters of ethyl acetate. Then, the solvent was distilled off using a rotary evaporator, followed by addition of 2.7 liters of ethyl acetate. The precipitated NaCl was filtered off and the ethyl acetate was distilled off, leaving an oily product. The oily product was dissolved in 2 liters of methylene chloride. 450 ml trifluoroacetic acid was added and the solution was stirred at room temperature (25° C.) for 100 minutes. The solvent and the trifluoroacetic acid was distilled off using a rotary evaporator, leaving an oily product. To the oily product 940 ml acetonitrile was added, followed by removal of the solvent by distillation using a rotary evaporator. This procedure—addition of 940 ml acetonitrile and removal of the solvent by distillation—was repeated 4 times. Then 2.2 liters of acetonitrile was added and the solution was stirred overnight at room temperature (25° C.), resulting in crystallization of the product. The suspension was filtered and the precipitate was dried under vacuum at room temperature for 3 days. The crystalline product 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate was stable for at least six months at 2° to 8° C.

Yield 162 g, 89% based on the amount of 2-[2-[2-[[2-[2-[2-[[2-[[4-(16-tert-butoxy-16-oxo-hexadecoxy)phenyl]sulfonylamino]pyrimidine-5-carbonyl]amino]ethoxy]ethoxy]acetyl]amino]-ethoxy]ethoxy]acetic acid used

### 2 Insulin Analogs

### 2.1 Insulin Analog 1

insulin analog 1 is based on human insulin with mutations in positions A14, B25, a removal of the amino acid at position B30 and an additional amino acid sequence TEGR (SEQ ID NO: 112) at the beginning of the A-chain:
Glu(A14): The amino acid at position 14 of the A-chain of human insulin (Y, tyrosine, Tyr) is substituted by glutamic acid (E, Glu),
Val(B25): The amino acid at position 25 of the B-chain of human insulin (F, phenylalanine, Phe) is substituted by valine (V, Val),
Des(B30): The amino acid at position 30 of the B-chain of human insulin is deleted.

The complete amino acid sequence of insulin analog 1 in view of A and B chain is:

A-chain:
(SEQ ID NO: 107)
TEGRGIVEQCCTSICSLEQLENYCN
B-chain:
(SEQ ID NO: 48)
FVNQHLCGSHLVEALYLVCGERGFVYTPK

The one intrachenar and the two interchenar disulfide bridges are in accordance with human insulin.

A prepro-insulin containing a signal peptide and the following proinsulin amino acid sequence was used:

(SEQ. ID NO: 108)
FVNQHLCGSHLVEALYLVCGERGFVYTPKTEGRGIVEQCCTSICSLEQL
ENYCN

A solution of the prepro-insulin, coming from a cation exchange chromatography capture step was adjusted to pH 8.5.

Endoproteinase Lys-C was added and the solution was stirred for two hours. The mixture was then purified by cation exchange chromatography followed by reversed phase chromatography. The solution with the product fractions was collected and lyophylisated. The resulting white powder contained insulin analog 1.

### 2.2 Insulin Analog 2

insulin analog 2 corresponds to insulin analog 1 with the exception that the TEGR (SEQ ID NO: 112)-group at the beginning of the A-chain is not present.

The complete amino acid sequence of insulin analog 2 in view of A and B chain is:

A-chain:
(SEQ ID NO: 47)
GIVEQCCTSICSLEQLENYCN
B-chain:
(SEQ ID NO: 48)
FVNQHLCGSHLVEALHLVCGERGFHYTPK

The one intrachenar and the two interchenar disulfide bridges are in accordance with human insulin.

### 3 Conjugate Synthesis

### 3.2 Conjugat of Insulin Analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate Solution in Acetonitrile—Conjugation in Water/Acetonitrile

37.5 g (12.4 mmol) insulin analog 1 from section 2.1 above was suspended in 938 ml water and the pH was adjusted with triethylamine to 11.1; 1313 ml acetonitrile was added. 600 ml (14.5 mmol) 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]-phenoxy]hexadecanoate solution in acetonitrile (20 g/litre) from section 1.1 above was added slowly under stirring at room temperature (25° C.) over 90 minutes to the solution of insulin analog 1 in water/acetonitrile. The pH was maintained at a value in the range of from 10.9 to 11 by addition of triethylamine. When an in-process-control by HPLC showed complete reaction (purity 54.4%), the pH was adjusted to 10.3 with 1N HCl and the acetonitrile in the solution was distilled off using a rotary evaporator. Then 1 liter water was added and the pH of the water solution was adjusted to 8.3 with 0.1 N HCl and 3.75 ml trypsin solution in water (11035 U/ml) was added and stirred overnight at room temperature (25° C.). At the time when the control with HPLC showed complete reaction (purity 50.7%), the solution was sent to chromatographic purification.

Yield in the water solution was calculated from HPLC data before purification as 50% based on the amount of insulin analog 1 used.

HPLC analytics were made while conjugation and after trypsin-cleavage with an Agilent 1100 HPLC at 210 nm:
Column: Phenomenex Gemini C6-Phenyl; 3 μm, 50×3 mm
Mobile Phase A: Water/trifluoroacetic acid 0.1%
Mobile Phase B: Acetonitrile/trifluoroacetic acid 0.1%
Gradient: 90% A/10% B to 12% A/82% B within 13 min the retention time in the chromatograms is 7.34 min after conjugation and before trypsin cleavage and 7.51 min after cleavage with trypsin.

The conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate had the structure shown in FIG. 3 (conjugate 1).

### 3.3 Conjugate of Insulin Analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate in Crystalline Form—Conjugation in Water/Acetonitrile/Tetrahydrofurane, Reaction in Solution

75 g (80 mmol) crystalline 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]-phenoxy]hexadecanoate from section 1.2 above was dissolved in 1.8 liters of tetrahydrofuran and 3.8 liters of acetonitrile (binder solution). 250 g (41.2 mmol) insulin analog 1 from section 2.1 above was suspended in 6.2 liters of water and the pH was adjusted with triethylamine to 10.3 to get insulin analog 1 dissolved. The binder solution was added to the insulin analog 1 solution under stirring at room temperature (25° C.). When an in-process-control with HPLC showed complete reaction (reaction turnover 99%; purity 80%), the organic solvents in the solution was distilled off using a rotary evaporator. Then the pH of the water solution was adjusted to a value in the range of from 8.0 to 8.5 with 0.1 N HCl. 3 ml trypsin solution in water (11035 U/ml) was added and stirred overnight at room temperature (25° C.) in order to remove the TEGR-group from the insulin analog 1's A-chain. When an in-process-control with HPLC showed complete reaction (reaction turnover 99.5%; purity 81%) the pH was adjusted to 6.5 with 1 N HCl and the solution was sent to chromatographic purification. Yield in the water solution was calculated from HPLC data before purification as 80% based on the amount of insulin analog 1 used.

Release analytics were made after chromatographic purification with an Agilent 1100 HPLC at 210 nm:
Column: Waters X-Select CSH C18; 2.5 μm, 150×4.6 mm
Mobile Phase A: Water/acetonitrile/trifluoroacetic acid 90%/10%/0.05%
Mobile Phase B: Water/acetonitrile/trifluoroacetic acid 10%/90%/0.05%
Gradient: 65% A/35% B to 10% A/90/B within 15 min Purity of the conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate was 98.0%, the retention time in the chromatograms was 7.65 min.

The conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate obtained in 3.3 had the structure shown in in FIG. 3 (conjugate 1).

### 3.4 Conjugate of Insulin Analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate in Crystalline Form—Conjugation in Water, Solid Phase Conjugation

250 g (41.2 mmol) insulin analog 1 from section 2.1 above was suspended in 10 liters of water and the pH was adjusted with triethylamine to a value in the range of from 10.6 to 10.8 to get the insulin analog 1 dissolved. 70 g (74.7 mmol) crystalline 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate from section 1.2 above was added to the aqueous solution of insulin analog 1 in 5×10 g portions and 4×5 g portions wherein the pH was kept at a value in the range of from 10.6 to 10.8 with triethylamine. When an in-process-control with HPLC showed complete reaction (reaction turnover 94%; purity 70%), the pH of the water solution was adjusted to a value in the range of from 8.2 to 8.4 with 1 N HCl. 3.25 ml trypsin solution in water (11035 u/ml) was added and stirred overnight at room temperature (25° C.) in order to remove the TEGR-group from the insulin's A-chain. When an in-process-control with HPLC showed complete reaction (reaction turnover 100%; purity 72%), the pH was adjusted to 6.5 with 1 N HCl and the solution was sent to chromatographic purification. Yield in the water solution was calculated from HPLC data before purification as 72% based on the amount of insulin analog 1 used.

Release analytics were made after chromatographic purification with an Agilent 1100 HPLC at 210 nm:
Column: Waters X-Select CSH C18; 2.5 μm, 150×4.6 mm
Mobile Phase A: Water/acetonitrile/trifluoroacetic acid 90%/10%/0.05%
Mobile Phase B: Water/acetonitrile/trifluoroacetic acid 10%/90%/0.05%
Gradient: 65% A/35% B to 10% A/90/B within 15 min

Purity of the conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate was 98.4%, the retention time in the chromatograms was 7.89 min.

The conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate obtained in 3.4 had the structure shown in in FIG. 3 (conjugate 1).

### 3.5 Conjugation of Insulin Analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate in Crystalline Form—Conjugation in Water/n-Propanol, Solid Phase Conjugation

99.49 g wet insulin analog 1 with a content of 23 g (3.8 mmol) insulin analog 1 from section 2.1 above was suspended in 688 ml water and the pH was adjusted with triethylamine to 10.9 to get the insulin analog 1 dissolved. 231 ml n-propanol was added and the pH was adjusted to 10.6. 6.25 g (6.68 mmol) crystalline 6-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate from section 1.2 above was added to the water/n-propanol solution in 2×2 g, 2×1 g and 1×250 mg portions wherein the pH was kept at a value in the range of from 10.6 to 10.8 with triethylamine. When an in-process-control with HPLC (reaction turnover 93%; purity 80%) showed complete reaction, the pH of the water/n-propanol solution was adjusted to 8.2 with 1 N HCl and 137.5 μl trypsin solution in water (18562 U/ml) was added and stirred overnight at room temperature in order the remove the TEGR-group from the insulin analog 1's A-chain. When an in-process-control with HPLC (reaction turnover 94%; purity 89%) showed complete reaction, the pH was adjusted to 6.8 with 1 N HCl and the solution was sent to chromatographic purification.

Yield in the water solution was calculated from HPLC data before purification as 84% based on the amount of insulin analog 1 used.

Release analytics were made after chromatographic purification with an Agilent 1100 HPLC at 210 nm:
Column: Waters X-Select CSH C18; 2.5 μm, 150×4.6 mm
Mobile Phase A: Water/acetonitrile/trifluoroacetic acid 90%/10%/0.05%
Mobile Phase B: Water/acetonitrile/trifluoroacetic acid 10%/90%/0.05%
Gradient: 65% A/35% B to 10% A/90/B within 15 min

Purity of the conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate was 96.0%, the retention time in the chromatograms was 8.12 min.

The conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate obtained in 3.5 had the structure shown in in FIG. 3 (conjugate 1).

### 3.6 Conjugate of Insulin Analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]-ethoxy]ethyl—Comparative Example

Tert-butyl ester of 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]-hexadecanoate was reacted with insulin analog 2 so that an amide bond was formed, followed by removal of the tert-butyl protective group as follows:

A solution of 400 mg of insulin analog 2 from section 2.2 above was suspended in 20 ml water and then 0.4 ml triethylamine was added. To the clear solution, 20 ml DMF and then 5 ml (17.04 mM in DMF) tert-butyl 16-[4-[[5-[2-[2-[2-[2-[2-[2-(2,5-dioxopyrrolidin-1-yl)oxy-2-oxo-ethoxy]ethoxy]ethylamino]-2-oxo-ethoxy]ethoxy]ethylcarbamoyl]pyrimidin-2-yl]sulfamoyl]-phenoxy]hexadecanoate) was added. The solution was stirred for 2 hours at room temperature. The reaction was analyzed with Waters UPLC H-class at 214 nm in a sodium chloride phosphate buffer.
Waters BEH300 10 cm.
Retention time insulin: 2.643 min.
Rentention time insulin conjugate 6.224 min.
The product was purified by HPLC with ÄKTA avant 25.
Kinetex 5 μm C18 100 A 250×21.2 mm. Column volume (CV) 88 ml.
Solvent A: 0.5% acetic acid in water
Solvent B: 0.5% acetic acid in water/acetonitrile 4: 6
Gradient: 80% A 20% B to 20% A 80% B in 10 CV

After lyophylisation of the product, the powder was dissolved in 2 ml trifluoroacetic acid. After one hour, the solution was neutralized with diluted sodium bicarbonate. The product was purified by HPLC with ÄKTA avant 25. Kinetex 5 μm C18 100 A 250×21.2 mm. Column volume (CV) 88 ml.
Solvent A: 0.5% acetic acid in water
Solvent B: 0.5% acetic acid in water/acetonitrile 4: 6
Gradient: 70% A 30% B to 30% A 70% B in 8 CV The reaction was analyzed with waters UPLC H-class at 214 nm in a sodium chloride phosphate buffer.
Waters BEH300 10 cm.
Retention time insulin conjugate: 5.121 min.

The solution was lyophilized and gave the desired product.
63 mg, 14% yield based on the amount of insulin analog 2 used.
Mass spec.: 6453.9 g/mol.

The conjugate of insulin analog 2 with 16-[4-[[5-[2-[2-[2-[2-[2-[2-(2,5-dioxopyrrolidin-1-yl) oxy-2-oxo-ethoxy] ethoxy]ethylamino]-2-oxo-ethoxy]ethoxy]ethylcarbamoyl]pyrimidin-2-yl]sulfamoyl]phenoxy]hexadecanoate obtained in 3.6 had the structure shown in in FIG. 3 (conjugate 1).

### 3.7 Conclusion

The yields of the synthesis routes from sections 3.2 to 3.6 in relation to the amount of insulin analog 1 and 2 respectively used are summarized as follows:

| | Conjugate of section ## | | | | |
|---|---|---|---|---|---|
| | | | | | 3.6 |
| | 3.2 | 3.3 | 3.4 | 3.5 | (comparative) |
| Yield [%] | 50 | 80 | 72 | 84 | 14 |

As can be derived from the yields summarized above, the inventive method of forming a conjugate of a sulfonamide and a polypeptide, here a conjugate of a specific sulfonamide and a specific insulin analog, enables higher overall yields in conjugate synthesis, i.e. yields of more than 20%, or more than 30%, or more than 40%, or more than 50%, based on the polypeptide/insulin analog used. The combined use of a specifically activated sulfonamide with a TEGR-protected insulin analog already increases the yield versus the comparative example at least at factor 3, i.e. the yield is at least 50%. Use of a solid form of the activated sulfonamide (in solid form, see 3.4 or 3.5 or solved in a solvent, see 3.3) further increases the yield to more than 70%. An optimized combination of solid phase reaction and a suitable solvent combination as in 3.5 enables yields of more than 80%.

II. In Vivo and In Vitro Testing

### 1. Production of Human Insulin and Insulin Analogs

Various insulins analogs with mutations e.g. at positions B16, B25 and/or A14 were generated. Table 1 provides an overview of the generated insulins.

TABLE 1
Generated analogs of human Insulin

| | | | | | Amino acid | SEQ ID | Amino acid | SEQ ID |
|---|---|---|---|---|---|---|---|---|
| | | | | | sequence | NO (A | sequence | NO (B- |
| Analog | Backbone | A14 | B16 | B25 | Chain A | chain) | Chain B | Chain) |
| WT | Human Insulin | Tyr | Tyr | Phe | GIVEQCCTSICS | 1 | FVNQHLCGSHLVEAL | 2 |
| | (wild-type) | | | | LYQLENYCN | | YLVCGERGFFYTPKT | |
| 2 | Glu(A14)Des | Glu | Tyr | Phe | GIVEQCCTSICS | 3 | FVNQHLCGSHLVEAL | 4 |
| | (B30)-Insulin | | | | LEQLENYCN | | YLVCGERGFFYTPK | |
| 3 | Leu(B16)Des | Tyr | Leu | Phe | GIVEQCCTSICS | 5 | FVNQHLCGSHLVEAL | 6 |
| | (B30)-Insulin | | | | LYQLENYCN | | LLVCGERGFFYTPK | |
| 4 | Gly(A21)Trp | Tyr | Trp | Phe | GIVEQCCTSICS | 7 | FVNQHLCGSHLVEAL | 8 |
| | (B16)Des(B30)- | | | | LYQLENYCG | | WLVCGERGFFYTPK | |
| | Insulin | | | | | | | |
| 5 | His(B16)Des | Tyr | His | Phe | GIVEQCCTSICS | 9 | FVNQHLCGSHLVEAL | 10 |
| | (B30)-Insulin | | | | LYQLENYCN | | HLVCGERGFFYTPK | |
| 6 | Val(B16)Des | Tyr | Val | Phe | GIVEQCCTSICS | 11 | FVNQHLCGSHLVEAL | 12 |
| | (B30)-Insulin | | | | LYQLENYCN | | VLVCGERGFFYTPK | |
| 7 | Ala(B25)-Insulin | Tyr | Tyr | Ala | GIVEQCCTSICS | 13 | FVNQHLCGSHLVEAL | 14 |
| | | | | | LYQLENYCN | | YLVCGERGFAYTPKT | |
| 8 | Ala(B25)Des | Tyr | Tyr | Ala | GIVEQCCTSICS | 15 | FVNQHLCGSHLVEAL | 16 |
| | (B30)-Insulin | | | | LYQLENYCN | | YLVCGERGFAYTPK | |
| 9 | Glu(B25)Des | Tyr | Tyr | Glu | GIVEQCCTSICS | 17 | FVNQHLCGSHLVEAL | 18 |
| | (B30)-Insulin | | | | LYQLENYCN | | YLVCGERGFEYTPK | |
| 10 | His(B25)Des | Tyr | Tyr | His | GIVEQCCTSICS | 19 | FVNQHLCGSHLVEAL | 20 |
| | (B30)-Insulin | | | | LYQLENYCN | | YLVCGERGFHYTPK | |
| 11 | Leu(B25)Des | Tyr | Ty | Leu | GIVEQCCTSICS | 21 | FVNQHLCGSHLVEAL | 22 |
| | (B30)-Insulin | | | | LYQLENYCN | | YLVCGERGFLYTPK | |
| 12 | Val(B25)Des | Tyr | Tyr | Val | GIVEQCCTSICS | 23 | FVNQHLCGSHLVEAL | 24 |
| | (B30)-Insulin | | | | LYQLENYCN | | YLVCGERGFVYTPK | |
| 13 | His(B16)His | Tyr | His | His | GIVEQCCTSICS | 25 | FVNQHLCGSHLVEAL | 26 |
| | (B25)Des(B30)- | | | | LYQLENYCN | | HLVCGERGFHYTPK | |
| | Insulin | | | | | | | |
| 14 | Gly(A21)Trp | Tyr | Trp | His | GIVEQCCTSICS | 27 | FVNQHLCGSHLVEAL | 28 |
| | (B16)His(B25)Des | | | | LYQLENYCG | | WLVCGERGFHYTPK | |
| | (B30)-Insulin | | | | | | | |
| 15 | Gly(A21)Trp | Tyr | Trp | Trp | GIVEQCCTSICS | 29 | FVNQHLCGSHLVEAL | 30 |
| | (B16)Trp(B25)Des | | | | LYQLENYCG | | WLVCGERGFWYTPK | |
| | (B30)-Insulin | | | | | | | |
| 16 | Glu(A14)His | Glu | His | Phe | GIVEQCCTSICS | 31 | FVNQHLCGSHLVEAL | 32 |
| | (B16)Des(B30)- | | | | LEQLENYCN | | HLVCGERGFFYTPK | |
| | Insulin | | | | | | | |
| 17 | Glu(A14)Gly | Glu | Trp | Phe | GIVEQCCTSICS | 33 | FVNQHLCGSHLVEAL | 34 |
| | (A21)Trp(B16)Des | | | | LEQLENYCG | | WLVCGERGFFYTPK | |
| | (B30)-Insulin | | | | | | | |
| 18 | Glu(A14)Ile(B16) | Glu | Ile | Phe | GIVEQCCTSICS | 35 | FVNQHLCGSHLVEALI | 36 |
| | Des(B30)- | | | | LEQLENYCN | | LVCGERGFFYTPK | |
| | Insulin | | | | | | | |
| 19 | Glu(A14)Val | Glu | Val | Phe | GIVEQCCTSICS | 37 | FVNQHLCGSHLVEAL | 38 |
| | (B16)Des(B30)- | | | | LEQLENYCN | | VLVCGERGFFYTPK | |
| | Insulin | | | | | | | |
| 20 | Glu(A14)Glu | Glu | Val | Phe | GIVEQCCTSICS | 39 | FVEQHLCGSHLVEAL | 40 |
| | (B3)Val(B16)Des | | | | LEQLENYCN | | VLVCGERGFFYTPK | |
| | (B30)-Insulin | | | | | | | |
| 21 | Glu(A14)His | Glu | Tyr | His | GIVEQCCTSICS | 41 | FVNQHLCGSHLVEAL | 42 |
| | (B25)Des(B30)- | | | | LEQLENYCN | | YLVCGERGFHYTPK | |
| | Insulin | | | | | | | |
| 22 | Glu(A14)Ile(B25) | Glu | Tyr | Ile | GIVEQCCTSICS | 43 | FVNQHLCGSHLVEAL | 44 |
| | Des(B30)- | | | | LEQLENYCN | | YLVCGERGFIYTPK | |
| | Insulin | | | | | | | |
| 23 | Glu(A14)Gly | Glu | Tyr | Trp | GIVEQCCTSICS | 45 | FVNQHLCGSHLVEAL | 46 |
| | (A21)Trp(B25)Des | | | | LEQLENYCG | | YLVCGERGFWYTPK | |
| | (B30)-Insulin | | | | | | | |
| 24 | Glu(A14)Val | Glu | Tyr | Val | GIVEQCCTSICS | 47 | FVNQHLCGSHLVEAL | 48 |
| | (B25)Des(B30)- | | | | LEQLENYCN | | YLVCGERGFVYTPK | |
| | Insulin | | | | | | | |
| 25 | Glu(A14)Gly | Glu | Tyr | Val | GIVEQCCTSICS | 49 | FVEQHLCGSHLVEAL | 50 |
| | (A21)Glu(B3)Val | | | | LEQLENYCG | | YLVCGERGFVYTPK | |
| | (B25)Des(B30)- | | | | | | | |
| | Insulin | | | | | | | |
| 26 | Glu(A14)Glu | Glu | Glu | His | GIVEQCCTSICS | 51 | FVNQHLCGSHLVEAL | 52 |
| | (B16)His(B25)Des | | | | LEQLENYCN | | ELVCGERGFHYTPK | |
| | (B30)-Insulin | | | | | | | |
| 27 | Glu(A14)His | Glu | His | Ala | GIVEQCCTSICS | 53 | FVNQHLCGSHLVEAL | 54 |
| | (B16)Ala(B25)Des | | | | LEQLENYCN | | HLVCGERGFAYTPK | |
| | (B30)-Insulin | | | | | | | |
| 28 | Glu(A14)His | Glu | His | His | GIVEQCCTSICS | 55 | FVNQHLCGSHLVEAL | 56 |
| | (B16)His(B25)Des | | | | LEQLENYCN | | HLVCGERGFHYTPK | |
| | (B30)-Insulin | | | | | | | |
| 29 | Glu(A14)Ile | Glu | Ile | Ile | GIVEQCCTSICS | 57 | FVNQHLCGSHLVEALI | 58 |
| | (B16)Ile(B25)Des | | | | LEQLENYCN | | LVCGERGFIYTPK | |
| | (B30)-Insulin | | | | | | | |
| 30 | Glu(A14)Glu | Glu | Ile | Ile | GIVEQCCTSICS | 59 | FVEQHLCGSHLVEALI | 60 |
| | (B3)Ile(B16)Ile | | | | LEQLENYCN | | LVCGERGFIYTPK | |
| | (B25)Des(B30)- | | | | | | | |
| | Insulin | | | | | | | |
| 31 | Glu(A14)Gly | Glu | Ile | Trp | GIVEQCCTSICS | 61 | FVEQHLCGSHLVEALI | 62 |
| | (A21)Glu(B3)Ile | | | | LEQLENYCG | | LVCGERGFWYTPK | |
| | (B16)Trp(B25)Des | | | | | | | |
| | (B30)-Insulin | | | | | | | |
| 32 | Glu(A14)Ile | Glu | Ile | Val | GIVEQCCTSICS | 63 | FVNQHLCGSHLVEALI | 64 |
| | (B16)Val(B25)Des | | | | LEQLENYCN | | LVCGERGFVYTPK | |
| | (B30)-Insulin | | | | | | | |
| 33 | Glu(A14)Gly | Glu | Ile | Val | GIVEQCCTSICS | 65 | FVEQHLCGSHLVEALI | 66 |
| | (A21)Glu(B3)Ile | | | | LEQLENYCG | | LVCGERGFVYTPK | |
| | (B16)Val(B25)Des | | | | | | | |
| | (B30)-Insulin | | | | | | | |
| 34 | Glu(A14)Leu | Glu | Leu | Ala | GIVEQCCTSICS | 67 | FVNQHLCGSHLVEAL | 68 |
| | (B16)Ala(B25)Des | | | | LEQLENYCN | | LLVCGERGFAYTPK | |
| | (B30)-Insulin | | | | | | | |
| 35 | Glu(A14)Val | Glu | Val | Ile | GIVEQCCTSICS | 69 | FVNQHLCGSHLVEAL | 70 |
| | (B16)Ile(B25)Des | | | | LEQLENYCN | | VLVCGERGFIYTPK | |
| | (B30)-Insulin | | | | | | | |
| 36 | Glu(A14)Gly | Glu | Val | Trp T | GIVEQCCTSICS | 71 | FVNQHLCGSHLVEAL | 72 |
| | (A21)Val(B16)Trp | | | | LEQLENYCG | | VLVCGERGFWYTPK | |
| | (B25)Des(B30)- | | | | | | | |
| | Insulin | | | | | | | |
| 37 | Glu(A14)Gly | Glu | Val | Trp | GIVEQCCTSICS | 73 | FVEQHLCGSHLVEAL | 74 |
| | (A21)Glu(B3)Val | | | | LEQLENYCG | | VLVCGERGFWYTPK | |
| | (B16)Trp(B25)Des | | | | | | | |
| | (B30)-Insulin | | | | | | | |
| 38 | Glu(A14)Val | Glu | Val | Val | GIVEQCCTSICS | 75 | FVNQHLCGSHLVEAL | 76 |
| | (B16)Val(B25)Des | | | | LEQLENYCN | | VLVCGERGFVYTPK | |
| | (B30)-Insulin | | | | | | | |
| 39 | Glu(A14)Glu | Glu | Val | Val | GIVEQCCTSICS | 77 | FVEQHLCGSHLVEAL | 78 |
| | (B3)Val(B16)Val | | | | LEQLENYCN | | VLVCGERGFVYTPK | |
| | (B25)Des(B30)- | | | | | | | |
| | Insulin | | | | | | | |
| 40 | Glu(A14)Gly | Glu | Val | Val | GIVEQCCTSICS | 79 | FVEQHLCGSHLVEAL | 80 |
| | (A21)Glu(B3)Val | | | | LEQLENYCG | | VLVCGERGFVYTPK | |
| | (B16)Val(B25)Des | | | | | | | |
| | (B30)-Insulin | | | | | | | |

### 2. Insulin Receptor Binding Affinity Assays/Insulin Receptor Autophosphorylation Assays

Insulin binding and signal transduction of various generated insulin analogs were determined by a binding assay and a receptor autophosphorylation assay.

A) Insulin Receptor Binding Affinity Assay

Insulin receptor binding affinity for the analogs listed in Table 1 was determined as described in Hartmann et al. (Effect of the long-acting insulin analogs glargine and degludec on cardiomyocyte cell signaling and function. Cardiovasc Diabetol. 2016; 15:96). Isolation of insulin receptor embedded plasma membranes (M-IR) and competition binding experiments were performed as previously described (Sommerfeld et al., PLoS One. 2010; 5(3): e9540). Briefly, CHO-cells overexpressing the IR were collected and re-suspended in ice-cold 2.25 STM buffer (2.25 M sucrose, 5 mM Tris-HCl pH 7.4, 5 mM MgCl₂, complete protease inhibitor) and disrupted using a Dounce homogenizer followed by sonication. The homogenate was overlaid with 0.8 STM buffer (0.8 M sucrose, 5 mM Tris-HCl pH 7.4, 5 mM MgCl₂, complete protease inhibitor) and ultra-centrifuged for 90 min at 100,000 g. Plasma membranes at the interface were collected and washed twice with phosphate buffered saline (PBS). The final pellet was re-suspended in dilution buffer (50 mM Tris-HCl pH 7.4, 5 mM MgCl₂, complete protease inhibitor) and again homogenized with a Dounce homogenizer. Competition binding experiments were performed in a binding buffer (50 mM Tris-HCl, 150 mM NaCl, 0.1% BSA, complete protease inhibitor, adjusted to pH 7.8) in 96-well microplates. In each well 2 μg isolated membrane was incubated with 0.25 mg wheat germ agglutinin polyvinyltoluene polyethylenimine scintillation proximity assay (SPA) beads. Constant concentrations of [125I]-labelled human insulin (100 pM) and various concentrations of respective unlabeled insulin (0.001-1000 nM) was added for 12 h at room temperature (23° C.). The radioactivity was measured at equilibrium in a microplate scintillation counter (Wallac Microbeta, Freiburg, Germany).”

The results of the insulin receptor binding affinity assays for the tested analogs relative to human insulin are shown in Table 2.

B) Insulin Receptor Autophosphorylation Assays (as a Measure for Signal Transduction)

In order to determine signal transduction of an insulin analog binding to insulin receptor B, autophosphorylation was measured in vitro.

CHO cells expressing human insulin receptor isoform B (IR-B) were used for IR autophosphorylation assays using In-Cell Western technology as previously described (Sommerfeld et al., PLoS One. 2010; 5(3): e9540). For the analysis of IGF1R autophosphorylation, the receptor was overexpressed in a mouse embryo fibroblast 3T3 Tet off cell line (BD Bioscience, Heidelberg, Germany) that was stably transfected with IGF1R tetracycline-regulatable expression plasmid. In order to determine the receptor tyrosine phosphorylation level, cells were seeded into 96-well plates and grown for 44 h. Cells were serum starved with serum-free medium Ham's F12 medium (Life Technologies, Darmstadt, Germany) for 2 h. The cells were subsequently treated with increasing concentrations of either human insulin or the insulin analog for 20 min at 37° C. After incubation the medium was discarded and the cells fixed in 3.75% freshly prepared para-formaldehyde for 20 min. Cells were permeabilised with 0.1% Triton X-100 in PBS for 20 min. Blocking was performed with Odyssey blocking buffer (LICOR, Bad Homburg, Germany) for 1 hour at room temperature. Anti-pTyr 4G10 (Millipore, Schwalbach, Germany) was incubated for 2 h at room temperature. After incubation of the primary antibody, cells were washed with PBS+0.1% Tween 20 (Sigma-Aldrich, St Louis, MO, USA). The secondary antimouse-IgG-800-CW antibody (LICOR, Bad Homburg, Germany) was incubated for 1 h. Results were normalized by the quantification of DNA with TO-PRO3 dye (Invitrogen, Karlsruhe, Germany). Data were obtained as relative units (RU).

The results of the insulin receptor autophosphorylation assays for the tested analogs relative to human insulin are shown in Table 2.

TABLE 2
Relative insulin receptor binding affinities and autophosphorylation activities
of tested analogs of human insulin (for the sequences, please see Table 1).

| | | | | | Insulin- | |
|---|---|---|---|---|---|---|
| | | | | | receptor | |
| | | | | | binding | Autophosphorylation |
| Analog | Backbone | A14 | B16 | B25 | affinity* | activity* |
| WT | Human Insulin (wild-type) | Tyr | Tyr | Phe | 1 | 1 |
| 2 | Glu(A14)Des(B30)-Insulin | Glu | Tyr | Phe | 1.05 | 0.87 |
| 3 | Leu(B16)Des(B30)-Insulin | Tyr | Leu | Phe | 0.24 | 0.34 |
| 4 | Gly(A21)Trp(B16)Des(B30)-Insulin | Tyr | Trp | Phe | 0.57 | 0.4 |
| 5 | His(B16)Des(B30)-Insulin | Tyr | His | Phe | nd | nd |
| 6 | Val(B16)Des(B30)-Insulin | Tyr | Val | Phe | nd | 0.32 |
| 7 | Ala(B25)-Insulin | Tyr | Tyr | Ala | 0.05 | 0.2 |
| 8 | Ala(B25)Des(B30)-Insulin | Tyr | Tyr | Ala | nd | 0.17 |
| 9 | Glu(B25)Des(B30)-Insulin | Tyr | Tyr | Glu | nd | nd |
| 10 | His(B25)Des(B30)-Insulin | Tyr | Tyr | His | 0.37 | 0.31 |
| 11 | Leu(B25)Des(B30)-Insulin | Tyr | Tyr | Leu | 0.01 | 0.06 |
| 12 | Val(B25)Des(B30)-Insulin | Tyr | Tyr | Val | 0.01 | 0.06 |
| 13 | His(B16)His(B25)Des(B30)-Insulin | Tyr | His | His | 0.11 | 0.1 |
| 14 | Gly(A21)Trp(B16)His(B25)Des(B30)-Insulin | Tyr | Trp | His | 0.4 | 0.35 |
| 15 | Gly(A21)Trp(B16)Trp(B25)Des(B30)-Insulin | Tyr | Trp | Trp | 0.43 | 0.38 |
| 16 | Glu(A14)His(B16)Des(B30)-Insulin | Glu | His | Phe | 0.36 | 0.29 |
| 17 | Glu(A14)Gly(A21)Trp(B16)Des(B30)-Insulin | Glu | Trp | Phe | 0.63 | 0.38 |
| 18 | Glu(A14)Ile(B16)Des(B30)-Insulin | Glu | Ile | Phe | 0.23 | 0.18 |
| 19 | Glu(A14)Val(B16)Des(B30)-Insulin | Glu | Val | Phe | nd | 0.32 |
| 20 | Glu(A14)Glu(B3)Val(B16)Des(B30)-Insulin | Glu | Val | Phe | 0.4 | 0.28 |
| 21 | Glu(A14)His(B25)Des(B30)-Insulin | Glu | Tyr | His | nd | nd |
| 22 | Glu(A14)Ile(B25)Des(B30)-Insulin | Glu | Tyr | Ile | 0.01 | 0.04 |
| 23 | Glu(A14)Gly(A21)Trp(B25)Des(B30)-Insulin | Glu | Tyr | Trp | 0.56 | 0.37 |
| 24 | Glu(A14)Val(B25)Des(B30)-Insulin | Glu | Tyr | Val | 0.01 | 0.04 |
| 25 | Glu(A14)Gly(A21)Glu(B3)Val(B25)Des(B30)-Insulin | Glu | Tyr | Val | 0.02 | 0.03 |
| 26 | Glu(A14)Glu(B16)His(B25)Des(B30)-Insulin | Glu | Glu | His | 0.01 | 0.07 |
| 27 | Glu(A14)His(B16)Ala(B25)Des(B30)-Insulin | Glu | His | Ala | | 0.11 |
| 28 | Glu(A14)His(B16)His(B25)Des(B30)-Insulin | Glu | His | His | 0.12 | 0.11 |
| 29 | Glu(A14)Ile(B16)Ile(B25)Des(B30)-Insulin | Glu | Ile | Ile | | 0.01 |
| 30 | Glu(A14)Glu(B3)Ile(B16)Ile(B25)Des(B30)-Insulin | Glu | Ile | Ile | 0** | 0.01 |
| 31 | Glu(A14)Gly(A21)Glu(B3)Ile(B16)Trp(B25)Des(B30)- | Glu | Ile | Trp | 0.12 | 0.13 |
| | Insulin | | | | | |
| 32 | Glu(A14)Ile(B16)Val(B25)Des(B30)-Insulin | Glu | Ile | Val | 0 | 0.04 |
| 33 | Glu(A14)Gly(A21)Glu(B3)Ile(B16)Val(B25)Des(B30)- | Glu | Ile | Val | 0.01 | 0.02 |
| | Insulin | | | | | |
| 34 | Glu(A14)Leu(B16)Ala(B25)Des(B30)-Insulin | Glu | Leu | Ala | 0.01 | 0.04 |
| 35 | Glu(A14)Val(B16)Ile(B25)Des(B30)-Insulin | Glu | Val | Ile | 0 | 0.01 |
| 36 | Glu(A14)Gly(A21)Val(B16)Trp(B25)Des(B30)-Insulin | Glu | Val | Trp | 0.17 | 0.23 |
| 37 | Glu(A14)Gly(A21)Glu(B3)Val(B16)Trp(B25)Des(B30)- | Glu | Val | Trp | 0.21 | 0.19 |
| | Insulin | | | | | |
| 38 | Glu(A14)Val(B16)Val(B25)Des(B30)-Insulin | Glu | Val | Val | 0 | 0.03 |
| 39 | Glu(A14)Glu(B3)Val(B16)Val(B25)Des(B30)-Insulin | Glu | Val | Val | 0 | 0.02 |
| 40 | Glu(A14)Gly(A21)Glu(B3)Val(B16)Val(B25)Des(B30)- | Glu | Val | Val | 0.01 | 0.01 |
| | Insulin | | | | | |

*relative to human insulin,
nd: not determined
**a value of 0 means that the binding affinity was below the detection limit

C) Conclusions

As can be derived from Table 2, various hydrophobic substitutions at positions B16 and/or B25 were tested (tryptophan, alanine, valine, leucine and isoleucine). Albeit to a different extent, all tested insulin analogs with hydrophobic substitutions at these positions showed a decrease of insulin receptor binding activity. As compared to tryptophan substitutions (see e.g. Analogs 4, 15 and 23), substitutions with aliphatic amino acid residues such as alanine, valine, leucine and isoleucine had a stronger impact on insulin receptor binding activity. The strongest effects were observed for valine, leucine and isoleucine, which are all branched-chain amino acid residues. Substitutions with isoleucine, valine and leucine resulted in a significant decrease of insulin receptor binding activity. Interestingly, insulin analogs with such substitutions at position B25 (such as valine, leucine or isoleucine substitution at B25, Analogs 11, 12, 22, 24, 25, 29, 30, 32, 33, 35 38, 39, 40) showed up to 6-fold enhancement in signal transduction than expected based on their IR-B binding affinities. Specifically, Leu(B25)Des(B30)-Insulin and Val(B25)Des(B30)-Insulin (Analogs 11 and 12, respectively) showed only 1% binding to insulin receptor B and 6% auto phosphorylation relative to human insulin. Similarly, a single leucine substitution at position B16 (Analog 3) also showed a similar enhancement in signal transduction albeit to a slightly lower extent. By comparison, with the exception of Analog 26, analogs bearing a histidine B25 substitution (Analogs 10, 13, 14, 21, 28) also showed reduced receptor binding, however a concomitant reduction in auto phosphorylation.

In some cases (Analogs 30, 32, 35, 38, 39), insulin receptor binding was 0% whilst still showing activity in the auto phosphorylation assay. All of these analogs have combinations of valine and/or isoleucine substitutions at positions B16 and B25 in common, suggesting that the combination is responsible for the further drop in insulin receptor binding. Insulins with no substitution at position B25 but with exchanges at position B16 exhibited slightly higher binding affinities in comparison to their autophosphorylation values (Analogs 3, 4, 16, 17, 18, 19, 20).

alanine in position B25 shows similar effects as valine, leucine or isoleucine substitution (analogs 11, 12, 22), although to a lower extent. The receptor binding affinity and autophosphorylation activity of analogs with valine, leucine or isoleucine substitution is lower than analogs with an alanine substitution.

3. Generation of Further Conjugates—In Vivo Testing—Evaluation of Pharmacokinetic Effects

Insulin conjugates 1 to 4 were prepared and tested. As a control, insulin conjugate 5 was prepared, which has been described in WO2018109162A1 [Norrman, Novo Nordisk].

The prepared insulin conjugates are summarized in the following table (Table 3). Further, insulin conjugates 1 to 4 are shown in FIGS. 3 to 6.

TABLE 3
Overview on Conjugates 1 to 5

| Insulin | | |
|---|---|---|
| conjugate | Insulin backbone* | Albumin binder/Sulfonamide |
| Conjugate 1 (see FIG. 3) | Glu(A14)Val(B25) Des(B30)-Insulin (Analog 24 in Table 1) | 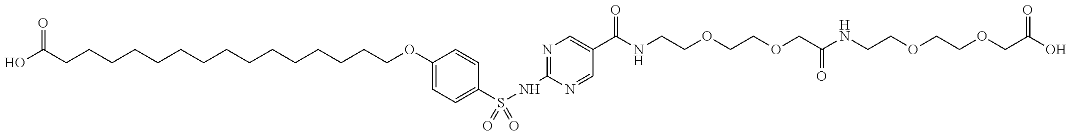 [SMILES: O=C(O)CCCCCCCCCCCCCCCOc1ccc(S(=O)(=O)Nc2ncc(C(=O)NCCOCCOCC(=O)NCCOCCOCC(=O)O)cn2)cc1] |
| Conjugate 2 (see FIG. 4) | Glu(A14)Val(B25) Des(B30)-Insulin (Analog 24 in Table 1) | 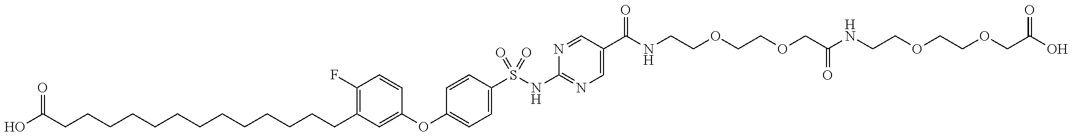 [SMILES: O=C(O)CCCCCCCCCCCCCc1cc(Oc2ccc(S(=O)(=O)Nc3ncc(C(=O)NCCOCCOCC(=O)NCCOCCOCC(=O)O)cn3)cc2)ccc1F] |
| Conjugate 3 (see FIG. 5) | Glu(A14)Glu(B3) Val(B16)Val(B25) Des(B30)-Insulin (Analog 39 in Table 1) | 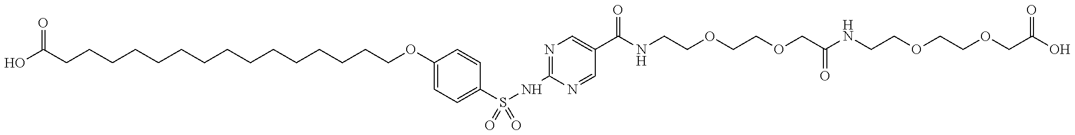 [SMILES: O=C(O)CCCCCCCCCCCCCCCOc1ccc(S(=O)(=O)Nc2ncc(C(=O)NCCOCCOCC(=O)NCCOCCOCC(=O)O)cn2)cc1] |
| Conjugate 4 (see FIG. 6) | Glu(A14)Ile(B25) Des(B30)-Insulin (Analog 22 in Table 1) | 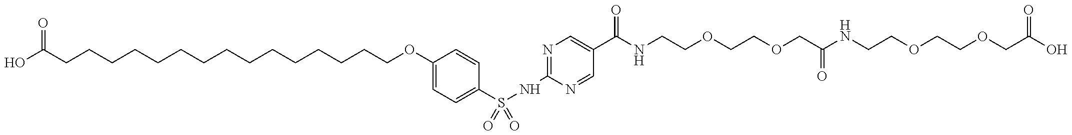 [SMILES: O=C(O)CCCCCCCCCCCCCCCOc1ccc(S(=O)(=O)Nc2ncc(C(=O)NCCOCCOCC(=O)NCCOCCOCC(=O)O)cn2)cc1] |
| Conjugate 5 | Glu(A14)His(B16) | Eicosandioyl-gammaGlu-OEG₂ |
| (described | His(B25)Des(B30)- | |
| in | Insulin | |
| WO2018109 | (Analog 28 in Table | |
| 162A1) | 1) | |

*for the sequence, see Table 1

Healthy, normoglycemic Gottingen mini pigs were used to evaluate the pharmacodynamic and pharmacokinetic effects of very long-acting insulin conjugates in vivo (pigs between 0.5-6 years were used with body weight ranges, depending on age, between ˜12-40 kg). The pigs were kept under standard laboratory animal housing conditions and were fed once daily with ad libitum access to tap water. After overnight fasting the pigs were treated with a single subcutaneous injection of a solution that contains either a placebo formulation or the respective insulin conjugate. The insulin conjugates 1˜4 as well as insulin conjugate 5 (described in WO2018109162A1 [Novo Nordisk, Norrman])) were tested.

Blood collection was performed via pre-implanted central venous catheters for determination of blood glucose, pharmacokinetics and additional biomarkers from K-EDTA plasma. Blood sampling started before the administration of the test item (baseline) and was repeated 1-4 times per day until study end. During the study, the animals were fed after the last blood sampling of the day. All animals were handled regularly and clinical signs were recorded at least twice on the day of treatment and once daily for the remaining duration of the study. The animals were monitored carefully for any clinical signs of hypoglycemia, including behavior, coat, urine and fecal excretion, condition of body orifices and any signs of illness. In case of severe hypoglycemia the investigator was allowed to offer food or infuse glucose solution intravenously (i.v.) in case food intake was not possible. After the last blood sampling, the animals were transported back to the animal housing facility.

A) Effects on Fasting Blood Glucose

Results are also shown in FIG. 1

TABLE 4
Effect on Blood Glucose

| | | | Maximal |
|---|---|---|---|
| | | FPG Duration of | Glucose |
| Insulin | Dose | Glucose lowering > | Lowering |
| conjugate | [nM/kg] | 15% vs Placebo [h] | [%] |
| Conjugate 1 | 30 | >128 | 45 |
| Conjugate 2 | 30 | >104 | 39 |
| Conjugate 3 | 30 | >152 | 30 |
| Conjugate 4 | 30 | >104 | 27 |
| Conjugate 5 | 18 | >128 | 62 |

B) Measurements on Pharmacokinetic Parameters

Results are also shown in FIG. 2

TABLE 5
Effect on Blood Glucose

| | Dose | PK tₘₐₓ | PK t_{1/2} |
|---|---|---|---|
| Conjugate | [nM/kg] | [h] | [h] |
| Conjugate 1 | 30 | 8 | 45 |
| Conjugate 2 | 30 | 20 | 38.9 |
| Conjugate 3 | 30 | 8 | 42.8 |
| Conjugate 4 | 30 | 14.7 | 45.2 |
| Conjugate 5 | 18 | 32 | 39 |

C) Conclusions

A single administration of insulin conjugate 4 (Ile(B25)), dosed at 30 nM/kg displayed a low to moderate glucose lowering effect with a flat profile up to 152 hours. Insulin conjugate 3, which contains mutations Val(B16) and Val(B25) displayed a flat profile of up to 152 hours with a moderate to medium glucose lowering effect. Furthermore, both insulin conjugates 1 and 2, containing the mutation Val(B25), lead to a stable glucose lowering effect without induction of hypoglycemia at a dose of 30 nM/kg. In contrast, insulin conjugate 5 (described in WO2018109162A1) was found to display a stronger glucose lowering effect with a less flat time-action profile compared to insulin conjugates 1-4 at a dose of only 18 nM/kg. Compound may have a higher risk for hypoglycemia.

Pharmacokinetic parameters show that insulin conjugates 1-4 display an earlier Tₘₐₓ in the range of 8-20 hours in combination with a plateau at Cₘₐₓ up to 50 hours. Because they display a terminal long t % in the range of 39-45 hours, a flat PK (pharmacokinetic) profile is achieved that is desired for once-weekly dosing due to the potentially reduced risk for hypoglycemic events.

### SHORT DESCRIPTION OF THE FIGURES

FIG. 1 shows blood glucose levels [% relative to placebo] on the y axis versus time on the x axis in hours after subcutaneous administration of insulin conjugates 1 to 5.

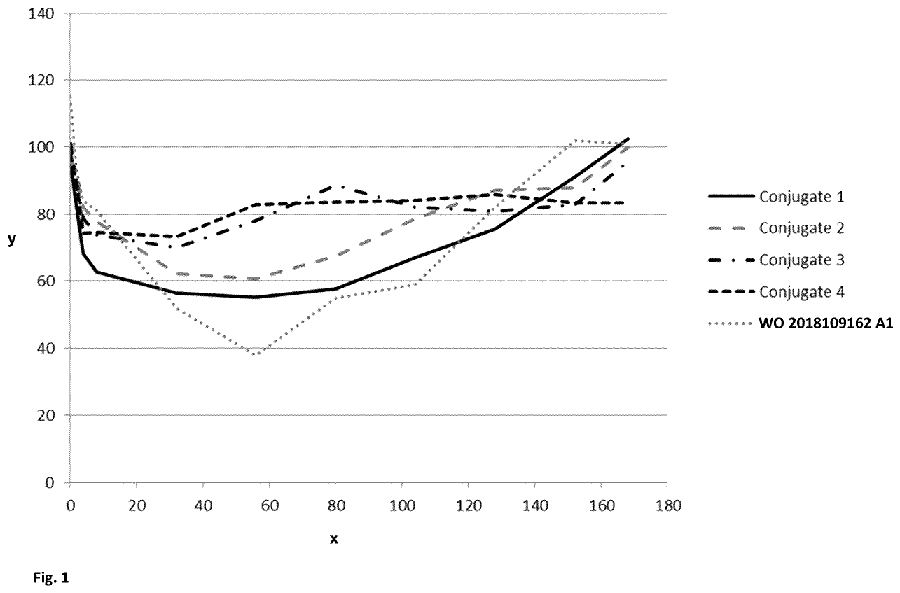

FIG. 2 shows normalized plasma concentration [ng/ml] on the y axis−time [h] on the x axis curves for insulin conjugates 1 to 5.

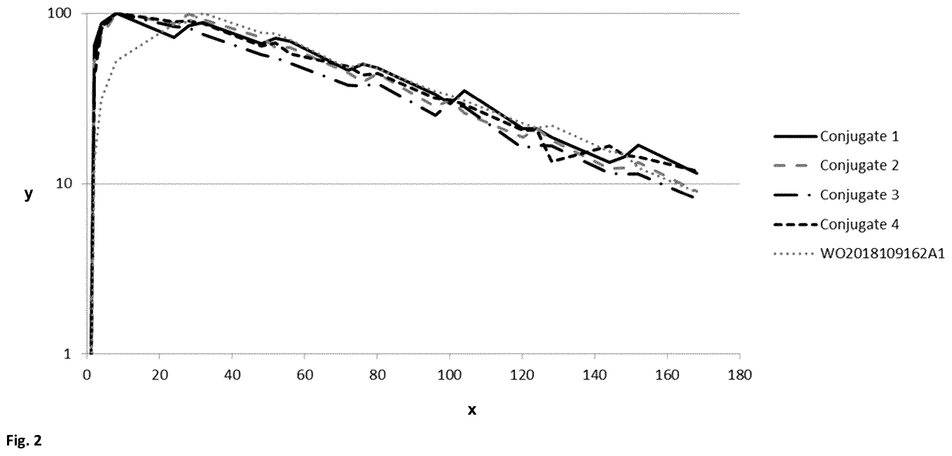

FIG. 3 shows insulin conjugate No. 1 (see Example 4 for more details). The sequences of the A chain (SEQ ID NO: 47) and the B chain (SEQ ID NO: 48) are indicated in three-letter-code, except for the last amino acid in the B chain (lysine at position B29). The structure of the lysine residue is shown. The lysine residue is covalently bound to the binder (via the epsilon amino acid of the lysine residue).

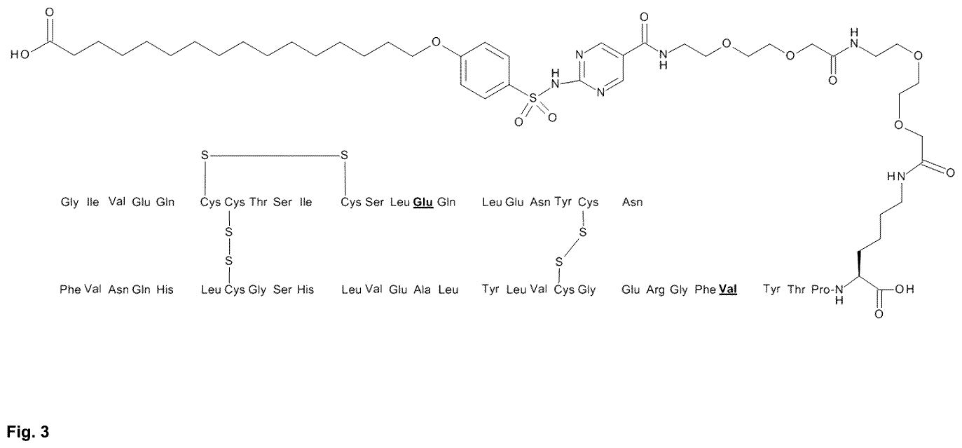

FIG. 4 shows insulin conjugate No. 2. The sequences of the A chain (SEQ ID NO: 47) and the B chain (SEQ ID NO: 48) are indicated in three-letter-code, except for the last amino acid in the B chain (lysine at position B29). The structure of the lysine residue is shown. The lysine residue is covalently bound to the binder (via the epsilon amino acid of the lysine residue).

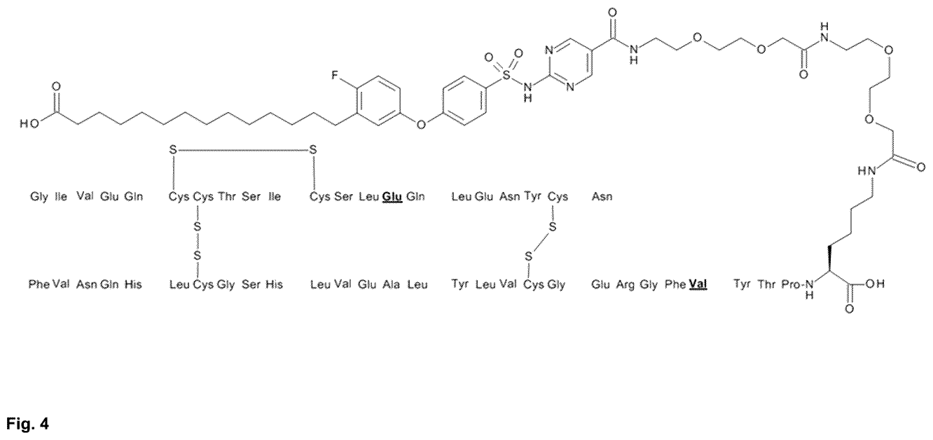

FIG. 5 shows insulin conjugate No. 3. The sequences of the A chain (SEQ ID NO: 77) and the B chain (SEQ ID NO: 78) are indicated in three-letter-code, except for the last amino acid in the B chain (lysine at position B29). The structure of the lysine residue is shown. The lysine residue is covalently bound to the binder (via the epsilon amino acid of the lysine residue).

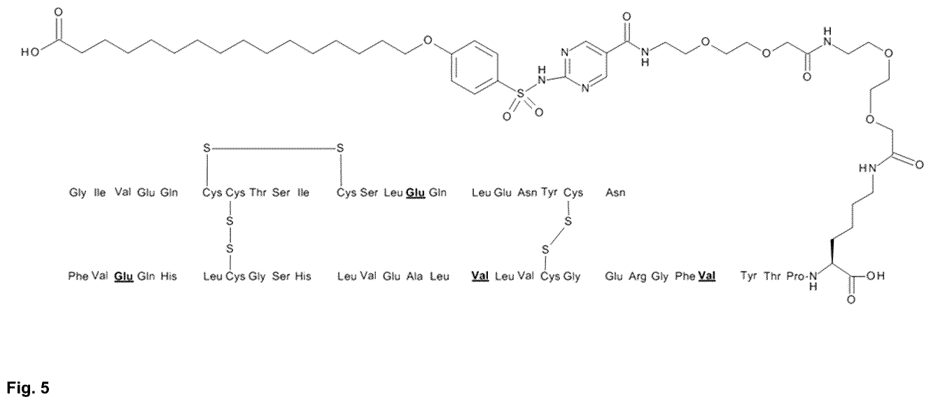

FIG. 6 shows insulin conjugate No. 4. The sequences of the A chain (SEQ ID NO: 43) and the B chain (SEQ ID NO: 44) are indicated in three-letter-code, except for the last amino acid in the B chain (lysine at position B29). The structure of the lysine residue is shown. The lysine residue is covalently bound to the binder (via the epsilon amino acid of the lysine residue).

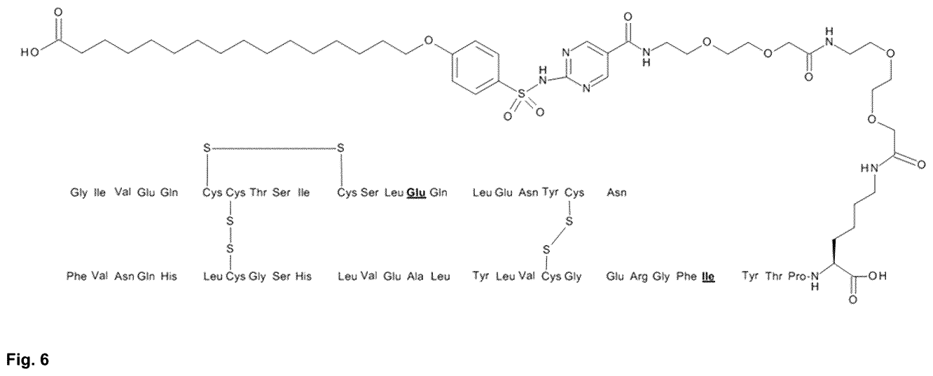

FIG. 7 shows the sequence of the insulin precursor (B-chain: SEQ ID NO: 48, N-terminally extended A-chain: SEQ ID NO: 107).

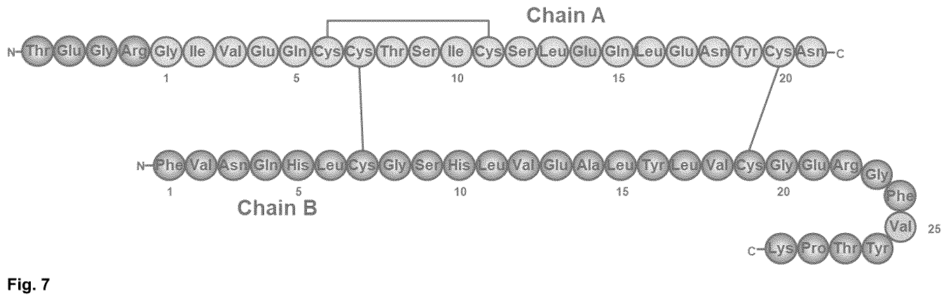

FIG. 8 shows the conjugate comprising the insulin precursor and a sulfonamide (B-chain: SEQ ID NO: 48, N-terminally extended A-chain: SEQ ID NO: 107).

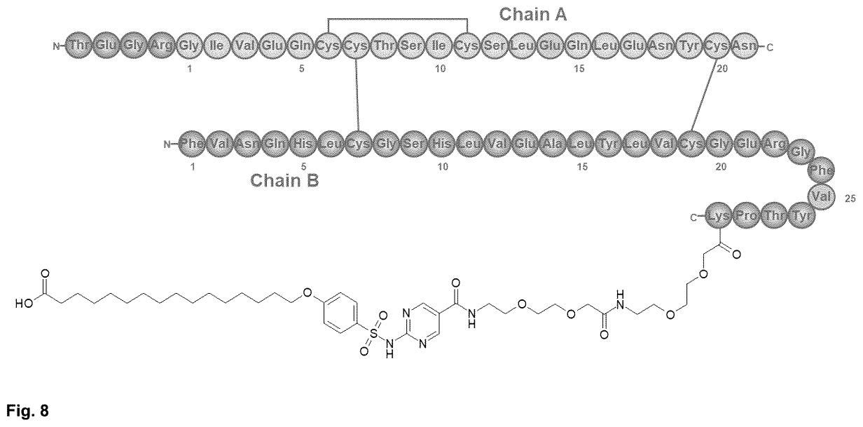

1. A method of forming a conjugate of a sulfonamide and a polypeptide, the method comprising:
a) Providing an activated sulfonamide, wherein the activated sulfonamide corresponds to Formula (I): 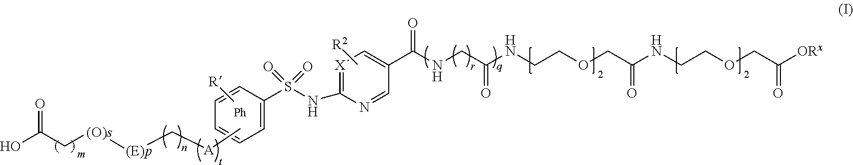
wherein
A is selected from the group consisting of oxygen atom, —CH₂CH₂— group, —OCH₂— group and —CH₂O— group;
E represents a —C₆H₃R— group with R being a hydrogen atom or a halogen atom, wherein the halogen atom is selected from the group consisting of fluorine, chlorine, bromine and iodine atom;
X represents a nitrogen atom or a —CH— group;
m is an integer in the range from 5 to 17;
n is zero or an integer in the range from 1 to 3;
p is zero or 1;
q is zero or 1;
r is an integer in the range from 1 to 6;
s is zero or 1;
t is zero or 1;
R¹ represents at least one residue selected from the group of hydrogen atom, halogen atom, C1 to C3 alkyl group and halogenated C1 to C3 alkyl group;
R² represents at least one residue selected from the group of hydrogen atom, halogen atom, C1 to C3 alkyl group and halogenated C1 to C3 alkyl group;
R^{x} represents an activation group;
wherein the combination of s being 1, p being zero, n being zero, A being an oxygen atom and t being 1 is excluded for Formula (I);
b) Providing an aqueous solution of a polypeptide having a free amino group, wherein the aqueous solution optionally comprises an alcohol;
c) Contacting the aqueous solution of b) with the activated sulfonamide of a); and
d) Reacting the activated sulfonamide with the polypeptide having a free amino group, obtaining a solution comprising the conjugate of a sulfonamide and a polypeptide, wherein the sulfonamide is covalently bonded to the polypeptide.

2. The method of claim 1, being a method of forming a conjugate of a sulfonamide and an insulin polypeptide, optionally wherein the activated sulfonamide is an activated albumin binder.

3. The method of claim 1 or 2, wherein contacting the aqueous solution of b) with the activated sulfonamide of a) according to step c) is done in that the activated sulfonamide of a) is added as a solution of the activated sulfonamide to the aqueous solution of b).

4. The method of any one of claims 1 to 3, wherein contacting the aqueous solution of b) with the activated sulfonamide of a) according to step c) is done in that the activated sulfonamide of a) is added in solid form to the aqueous solution of b), or at least partially in crystalline form, or at least 90 weight-% in crystalline form;
preferably wherein step d) comprises:
d.1) Reacting the activated sulfonamide with a precursor of the polypeptide having a free amino group at a pH in the range from 9 to 12, or in the range from 9.5 to 11.5 or in the range from 10 to 11, obtaining a pre-conjugate comprising the sulfonamide and the precursor of the polypeptide, wherein the sulfonamide is covalently bonded to the precursor of the polypeptide by an amide bond C(═O)—NH— formed between the —C(═O)—O(R) of the (activated) sulfonamide of Formula (I) and the amino group of the precursor of the polypeptide;
d.2) Enzymatic digestion optionally at a pH in the range below 9, or at a pH in the range of 7 to 9, of the precursor of the polypeptide of the pre-conjugate obtained according to d.1), obtaining a solution comprising the conjugate of the sulfonamide and the polypeptide.

5. A conjugate obtained or obtainable from the method of any one of claims 1 to 4.

6. An N-terminally extended insulin A-chain comprising from N- to C-terminus:
(a) a linker peptide, and
(b) an insulin A-chain,
wherein said N-terminally extended insulin A-chain comprises a cleavage site for trypsin between the last amino acid of the linker peptide and the first amino acid of the A-chain.

7. An insulin precursor comprising the N-terminally extended insulin A-chain of claim 6 and an insulin B-chain.

8. A procedure for crystallizing an activated sulfonamide corresponding to Formula (I) 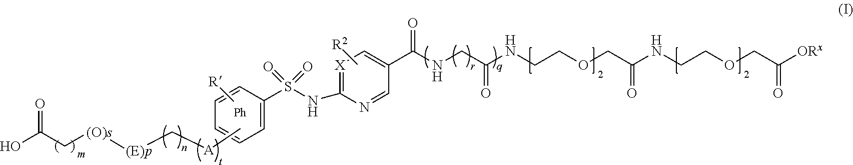
wherein A, E, X, m, n, p, q, r, s, t, R¹, R² and R^{x} have the meaning as defined in claim 1, comprising
A) Providing a solution comprising the activated sulfonamide and an organic solvent;
B) Removing the organic solvent at least partially, obtaining a phase of the activated sulfonamide having a reduced amount of the organic solvent compared to the solution provided in A);
C) Adding organic solvent to the phase obtained in B) obtaining a solution of the activated sulfonamide; and
D) Repeating step B) with the solution obtained in C) obtaining a phase of the activated sulfonamide having a reduced amount of the organic solvent compared to the solution obtained in C);
E) Optionally repeating steps C) and D) at least one further time.

9. A solid, optionally crystalline, form of the activated sulfonamide corresponding to Formula (I) 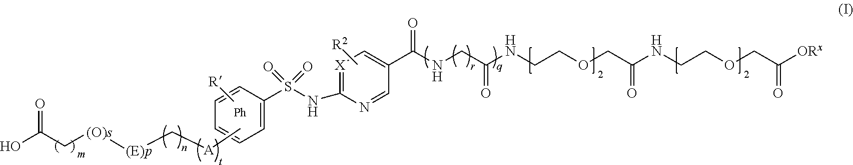
wherein A, E, X, m, n, p, q, r, s, t, R¹, R² and R^{x} have the meaning as defined in claim 1.

10. A process for generating a conjugate of an albumin binder and a mature insulin, said process comprising
a) Providing a proinsulin comprising from N- to C-terminus an insulin B-chain, a linker peptide and an insulin A-chain,
b) Cleaving the proinsulin provided in step a) with a first protease between the last amino acid of the insulin B-chain and the first amino acid of the linker peptide, thereby generating an insulin precursor, said insulin precursor comprising the insulin B-chain and an N-terminally extended A-chain comprising the linker peptide and the A-chain,
c) contacting said insulin precursor with an albumin binder, wherein the albumin binder comprises a functional group capable of binding to albumin, thereby generating a conjugate of an albumin binder and the insulin precursor,
d) Cleaving the N-terminally extended A-chain of said insulin precursor comprised by the conjugate with a second protease between the last amino acid of the linker peptide and the first amino acid of the A-chain, thereby generating a conjugate of a sulfonamide and a mature insulin.

11. The process of claim 10, wherein the last amino acid of the insulin B-chain is a lysine residue.

12. The process of claim 10 or 11, wherein the linker peptide has a length of at least two amino acid residues, for example wherein the linker peptide has length of 2 to 30 amino acid residues, such as a length of 4 to 9 amino acid residues.

13. The process of any one of claims 10 to 12, wherein the first amino acid of the linker peptide is a threonine residue, phenylalanine residue, a glutamine residue, a glutamic acid residue, an asparagine residue or an aspartic acid residue and/or wherein the last amino acid of the linker peptide is an arginine residue.

14. A proinsulin comprising from N- to C-terminus:
a) an insulin B-chain,
b) a linker peptide, and
c) an insulin A-chain,
wherein said proinsulin comprises a cleavage site for endoproteinase Lys-C between the last amino acid of the insulin B-chain and the first amino acid of the linker peptide and a cleavage site for trypsin between the last amino acid of the linker peptide and the first amino acid of the insulin A-chain.

15. The process of any one of claims 10 to 13, or the proinsulin of claim 14, wherein the linker peptide comprises the following sequence
(SEQ ID NO: 106)
Xaa1-Xaa2-Xaa3-Xaa4-Xaa5-Xaa6-Xaa7-Xaa8-Arg
wherein
Xaa1 is any naturally occurring amino acid residue,
Xaa2 is any naturally occurring amino acid residue, or wherein Xaa2 is absent,
Xaa3 is any naturally occurring amino acid residue or wherein Xaa3 is absent,
Xaa4 is any naturally occurring amino acid residue, or wherein Xaa4 is absent,
Xaa5 is any naturally occurring amino acid residue, or wherein Xaa5 is absent,
Xaa6 is any naturally occurring amino acid residue, or wherein Xaa6 is absent,
Xaa7 is any naturally occurring amino acid residue, or wherein Xaa7 is absent, and
Xaa8 is any naturally occurring amino acid residue, or wherein Xaa8 is absent.

16. The process of any one of claims 10 to 13 and 15, or the proinsulin of claim 14, wherein the linker peptide has the sequence TEGR (SEQ ID NO: 112).
